SEQUENCE LISTING (3 sequences)

SEQ ID NO: 1 - DNA, 20 bp, Artificial Sequence
  Synthetic Construct
tgtgtgtgtgtgtgtgtgtg

SEQ ID NO: 2 - DNA, 20 bp, Artificial Sequence
  Synthetic Construct
cacacacacacacacacaca

SEQ ID NO: 3 - protein, 5 aa, Artificial Sequence
  Synthetic Construct
  VARIANT 1, 3: Xaa = Asp or Glu
  VARIANT 5: Xaa = Ser or Gly
XWXYX

(19) United States
(12) Patent Application Publication
(10) Pub. No.: US 2007/0218072 A1
(43) Pub. Date: Sep. 20, 2007

(54) METHODS OF IMPROVING HEALTH-RELATED QUALITY OF LIFE IN INDIVIDUALS WITH SYSTEMIC LUPUS ERYTHEMATOSUS

(76) Inventors: Vibeke Strand, Portola Valley, CA (US); Matthew D. Linnik, Solana Beach, CA (US); Tenshang Joh, Encinitas, CA (US)

Correspondence Address: MORRISON & FOERSTER LLP, 755 PAGE MILL RD, PALO ALTO, CA, 94304-1018, US

(21) Appl. No.: 11/562,174

(22) Filed: Nov. 21, 2006

Related U.S. Application Data
(63) Continuation of application No. 10/748,541, filed on Dec. 29, 2003.
(60) Provisional application No. 60/436,906, filed on Dec. 27, 2002.
(60) Provisional application No. 60/478,128, filed on Jun. 11, 2003.

(51) Int. Cl. A61K 39/385; A61K 39/00

(52) U.S. Cl. 424/185.1; 424/184.1; 424/193.1

(57) ABSTRACT

The invention provides methods for stabilizing or improving the health-related quality of life in individuals with SLE, and methods of selecting individuals suitable for such treatment. One method of stabilizing or improving the health-related quality of life of an individual with SLE involves the administration of an effective amount of dsDNA epitope, such as in the form of an epitope-presenting carrier or an epitope-presenting valency platform molecule like LJP 394, to the individual. The invention further provides a method of stabilizing or improving the health-related quality of life of an individual with SLE involving the reduction of the level of SLE-associated antibodies in the individual, optionally through administration of a dsDNA epitope to the individual. In addition, methods of screening patients are provided. Kits useful in the methods of the invention are also provided.

### CROSS-REFERENCE TO RELATED APPLICATIONS

This application is a continuation of U.S. application Ser. No. 10/748,541, filed Dec. 29, 2003, which claims the priority benefit of U.S. Provisional Application No. 60/436,906, filed Dec. 27, 2002, and U.S. Provisional Application No. 60/478,128, filed Jun. 11, 2003, the contents of each of which are hereby incorporated by reference into the present disclosure.

### TECHNICAL FIELD

This invention relates to the field of antibody-mediated pathologies such as lupus. More particularly, the invention relates to methods of improving or stabilizing the health-related quality of life of individuals with systemic lupus erythematosis.

### BACKGROUND OF THE INVENTION

Systemic lupus erythematosus (SLE) is characterized by multisystem organ involvement and variable disease course including flares and remissions. Renal disease is a primary cause of morbidity and mortality in SLE patients (Pistiner M, et al. (1991) Semin Arthritis Rheum 21:55-64, Hochberg M C, et al. (1985) Medicine 64:285-295, Dubois E L, et al. (1964) JAMA 190:104-11, Vitali C, et al. (1992) Clin Exp Rheumatol 10:527-39). In patients with SLE renal disease, high levels of anti-double stranded DNA antibodies (anti-dsDNA) correlate with active glomerulonephritis. A pathogenic role is suggested as these antibodies can be eluted from diseased glomeruli (Winfield J B, et al. (1977) J Clin Invest 59:90-6, Hahn, B. (1998) N Engl J Med 338:1359-68, Vlahakos D V, et al. (1992) Kidney Int 41:1690-700, Ehrenstein M R, et al. (1995) Kidney Int 48:705-11, Rothfield N F, et al. (1967) J Clin Invest 46:1785-94, Lefkowith J B, et al. (1996) J Clin Invest 98:1373-80). Significant increases in anti-dsDNA levels are associated with increased SLE disease activity; sustained reductions in antibody levels have been associated with improved treatment outcomes (Borg E J, et al. (1990) Arthritis Rheum, 33:634-43, Swaak A J G, et al. (1986) Ann Rheum Dis 45:359-66, Bootsma H, et al. (1995) Lancet 345:1595-9).

Although overall patient prognosis in SLE has improved, treatment regimens are not ideal and lupus nephritis continues to be associated with relatively poor overall survival as compared to individuals without renal involvement in lupus (Seleznick et al. (1991) Semin. Arthritis Rheum. 21:73-80). Acute episodes of nephritis are usually treated with high dose corticosteroids and/or immunosuppressive agents, typically cyclophosphamide, azathioprine, or recently mycophenolate mofetil. Poor tolerability, insufficient efficacy, and toxicity associated with these treatments limit their use, creating a need for alternative therapies (Klippel J H, et al. (1990) JAMA 263:1812-5, Ortmann R A, et al. (2000) Rheum Dis Clin North Am 26:363-75).

Synthetic double-stranded oligonucleotides (dsON) have been shown to cross-react with anti-dsDNA antibodies (U.S. Pat. No. 5,276,013). The use of dsON conjugated with non-immunogenic carriers, also referred to as platforms, has been proposed for a therapeutic approach for the treatment of SLE. For example, a tetrakis conjugate, LJP 249, composed of four dsON attached to a poly(ethylene glycol) valency platform was used to demonstrate tolerance in an immunized mouse model system (Jones et al. (1994) Bioconjugate Chem. 5:390-399).

LJP 394 (abetimus sodium; also known as Riquent™), composed of 4 deoxynucleotide sequences bound to a triethylene glycol backbone, is a non-immunogenic, immunomodulatory agent, that selectively reduces anti-dsDNA titers in murine models of SLE and in patients with SLE (Plunkett et al. (1995) Lupus 4:S99, Coutts S M, et al. (1996) Lupus 5:158-9, Weisman M H (1997) J Rheumatol 24:314-38, Furie R A, et al. (2001) J Rheumatol 28:257-65). LJP 394 has been shown to induce antigen-specific B-cell tolerance in mice and rats, believed to occur by crosslinking anti-dsDNA antibodies on the surface of B cells resulting in anergy or apoptosis (Hartley S B, et al. (1993) Cell 72:325-35, Finkelman F D, et al. (1995) J Exp Med 181:515-25, Norvell A, et al. (1995) J Immunol 154:4404-13).

International Patent Application No. WO 01/41813 discloses methods of identifying lupus patients, including those with lupus nephritis, with high affinity anti-dsDNA antibodies and treatment of such patients with LJP 394. Other references discuss LJP394 in the context of a potential therapeutic agent for lupus. See Strand (2001) Lupus 10:216-221; Wallace (2001) Expert Opinion of Investigational Drugs 10:111-117; Furie et al. (2001) J. Rheumatol. 28:257-265.

Other literature describes methods which may be used in the treatment of SLE, including methods of reducing levels of circulating antibodies by inducing B cell tolerance, including, but not limited to, U.S. Pat. Nos. 5,276,013; 5,391,785; 5,786,512; 5,726,329; 5,552,391; 5,268,454; 5,606,047; 5,633,395; 5,162,515; U.S. Ser. No. 08/118,055 (U.S. Pat. No. 6,060,056); U.S. Ser. Nos. 60/088,656 and 60/103,088 (U.S. Ser. No. 09/328,199 and PCT App. No. PCT/US99/13194). See also U.S. Pat. No. 6,022,544.

All references cited herein, including patents, patent applications and publications, are hereby incorporated by reference in their entirety.

### BRIEF SUMMARY OF THE INVENTION

In one aspect, the invention provides a method of stabilizing or improving the health-related quality of life in an individual with systemic lupus erythematosus (SLE), comprising administering to the individual an effective amount of a dsDNA epitope which specifically binds to an SLE-associated antibody (generally, an antibody which specifically binds to double-stranded DNA (an anti-dsDNA antibody), although as is known in the art, and described herein, such antibodies may also bind single-stranded DNA and/or mimetics or analogs of double-stranded DNA) from the individual, wherein the administration of the dsDNA epitope results in a stabilization of or improvement in the individual's health-related quality of life. In some embodiments, the dsDNA epitope is administered such that there is a sustained reduction of anti-dsDNA antibodies in the individual, wherein the sustained reduction is at least about 10% below baseline in the individual (for example, a value of 100 at baseline would drop at least about 10% to about 90). In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years, since SLE is a chronic disease. In some embodiments, the dsDNA epitope is the double-stranded polynucleotide 5′-GTGTGTGTGTGTGTGTGTGT-3′(SEQ ID NO:1) in combination with its complementary strand, particularly the sequence 3′-CACACACACACACACACACA-5′(SEQ ID NO:2), or one of the single-stranded polynucleotides 5′-GTGTGTGTGTGTGTGTGTGT-3′(SEQ ID NO:1) or 3′-CACACACACACACACACACA-5′(SEQ ID NO:2). The dsDNA epitope is optionally administered in the form of an epitope-presenting carrier. In some embodiments, the stabilization or improvement in the individual's health-related quality of life occurs before, during, or after a renal flare. In some embodiments the dsDNA epitope is administered to the individual for more than about 16 weeks.

In another aspect, the invention provides a method of stabilizing or improving the health-related quality of life in an individual with SLE, comprising administering to the individual an effective amount of a conjugate comprising (a) a non-immunogenic valency platform molecule and (b) two or more double-stranded DNA (dsDNA) epitopes which specifically bind to an antibody from the individual which specifically binds to double-stranded DNA, wherein the administration of the conjugate results in a stabilization of or improvement in the individual's health-related quality of life. In some embodiments, the conjugate is administered such that there is a sustained reduction of anti-dsDNA antibodies in the individual, wherein the sustained reduction is at least about 10% below baseline in the individual. In some embodiments, the sustained reduction is maintained for more than 16 weeks. In some embodiments, the sustained reduction is maintained for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years, since SLE is a chronic disease. In some embodiments, the dsDNA epitope is optionally administered as the epitope-presenting valency platform molecule LJP 394 (Jones et al. (1995) J. Med. Chem. 38:2138-2144). In some embodiments, the stabilization or improvement in the individual's health-related quality of life occurs before, during, or after a renal flare.

In another aspect, the invention provides a method of stabilizing or improving the health-related quality of life of an individual with systemic lupus erythematosus, comprising reducing the level of circulating SLE-associated antibodies in the individual, wherein reducing the level of circulating SLE-associated antibodies in the individual results in a stabilization of or improvement in the individual's health-related quality of life. In some embodiments, the method comprises administering to the individual an effective amount of an agent (such as a dsDNA epitope) that reduces the level of circulating SLE-associated antibodies in the individual. In some embodiments, the agent is administered such that there is a sustained reduction of anti-dsDNA antibodies in the individual, where the sustained reduction is at least about 10% below baseline in the individual. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years.

In some embodiments, the level of circulating SLE-associated antibodies in the individual is reduced by administration of an effective amount of a dsDNA epitope, such as the double-stranded polynucleotide 5′-GTGTGTGTGTGTGTGTGTGT-3′(SEQ ID NO:1) and its complementary strand, particularly the sequence 3′-CACACACACACACACACACA-5′(SEQ ID NO:2) or one of the single-stranded polynucleotides 5′-GTGTGTGTGTGTGTGTGTGT-3′(SEQ ID NO:1) or 3′-CACACACACACACACACACA-5′(SEQ ID NO:2) to the individual. In another embodiment, the invention provides a method of stabilizing or improving the health-related quality of life of an individual with SLE, comprising reducing the level of circulating SLE-associated antibodies in the individual by administering to the individual an effective amount of an epitope-presenting valency platform molecule, such as LJP 394. In some embodiments, administration of an effective amount of the dsDNA epitope results in a sustained reduction of anti-dsDNA antibodies in the individual, wherein the sustained reduction is at least about 10% below baseline in the individual. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years. In some embodiments, the stabilization or improvement in the individual's health-related quality of life occurs before, during, or after a renal flare.

In still another aspect, the invention provides a method of stabilizing or improving the health-related quality of life in an individual with SLE, comprising administering to the individual an effective amount of a mimetic of double-stranded DNA that specifically binds to an SLE-associated antibody (i.e., an anti-dsDNA antibody) from the individual, and wherein the administration of the mimetic results in a stabilization of or improvement in the individual's health-related quality of life.

In yet another aspect, the invention provides a method of stabilizing or improving the health-related quality of life in an individual with SLE before, during, or following a renal flare, comprising administering to the individual an effective amount of an agent (such as, a dsDNA epitope that specifically binds to an anti-dsDNA antibody from the individual) that reduces SLE-associated antibodies in the individual. In some embodiments, the administration of the agent results in the sustained reduction of anti-dsDNA antibodies in the individual, wherein the sustained reduction is at least about 10% below baseline in the individual. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years.

In still another aspect, the invention provides a method of stabilizing or improving the health-related quality of life of an individual with systemic lupus erythematosus, comprising effecting a sustained reduction of anti-dsDNA antibodies in the individual. In some embodiments, the sustained reduction is effected by the administration of an agent (such as, a dsDNA epitope that specifically binds to an anti-dsDNA antibody from the individual) that reduces the level of circulating anti-dsDNA antibodies in the individual. In some embodiments, the agent is administered such that there is a sustained reduction of anti-dsDNA antibodies in the individual, where the sustained reduction is at least about 10% below baseline in the individual. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years.

In a further aspect, the invention provides a method of stabilizing or improving the health-related quality of life in an individual with SLE prior to or following a renal flare, comprising effecting a sustained reduction of anti-dsDNA antibodies in the individual. In some embodiments, the sustained reduction is effected by the administration of an agent (such as, a dsDNA epitope that specifically binds to an anti-dsDNA antibody from the individual) that reduces the level of circulating anti-dsDNA antibodies in the individual. In some embodiments, the administration of the agent results in the sustained reduction of anti-dsDNA antibodies in the individual, wherein the sustained reduction is at least about 10% below baseline in the individual. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years.

In an additional aspect, the invention provides a method of stabilizing or improving the health-related quality of life in an individual following a renal flare, comprising reducing the level of circulating anti-dsDNA antibodies in the individual.

In a further aspect, the invention provides a method of stabilizing or improving the health-related quality of life in an individual with systemic lupus erythematosus (SLE), comprising effecting a sustained reduction of anti-dsDNA antibodies in the individual for a period of more than about 16 weeks.

In still another aspect, the invention provides a method of stabilizing or improving the health-related quality of life in an individual following a renal flare, comprising effecting a sustained reduction of anti-dsDNA antibodies in the individual for a period of more than about 16 weeks.

In another aspect, the invention provides a method of stabilizing or improving the health-related quality of life of an individual with SLE, comprising administering to the individual for a period of more than about 16 weeks an effective amount of a dsDNA epitope which specifically binds to an anti-dsDNA antibody from the individual.

In a further aspect, the invention provides a method of stabilizing or improving the health-related quality of life of an individual with SLE following a renal flare, comprising administering to the individual for a period of more than about 16 weeks an effective amount of a dsDNA epitope which specifically binds to an anti-dsDNA antibody from the individual.

In another aspect, the invention provides a method of stabilizing or improving the health-related quality of life in an individual with SLE comprising the steps of selecting an individual for receiving or continuing to receive treatment based on the individual's need for a stabilized or improved health-related quality of life, and administering a treatment to the selected individual, wherein administration of the treatment effects a sustained reduction of anti-dsDNA antibodies in the individual.

In still another aspect, the invention provides a method of stabilizing or improving the health-related quality of life in an individual having SLE comprising the steps of selecting an individual to receive or continue to receive a dsDNA epitope based on the affinity of the dsDNA epitope for an anti-dsDNA antibody in the individual, and administering the dsDNA epitope to the selected individual, wherein administration of the dsDNA epitope stabilizes or improves the health-related quality of life in an individual.

In an additional aspect, the invention provides a kit comprising a dsDNA epitope which specifically binds to an anti-dsDNA antibody from an individual with SLE, and instructions comprising a description of the administration of the dsDNA epitope to an individual to stabilize or improve the health-related quality of life in the individual.

In still another aspect, the invention provides a kit comprising a dsDNA epitope which specifically binds to an anti-dsDNA antibody from an individual with SLE, and instructions comprising a description of the selection of an individual suitable for receiving treatment by administration of the dsDNA epitope based on the low health-related quality of life of the individual.

With respect to the aspects of the invention, in some embodiments, the sustained reduction of anti-dsDNA antibodies in the individual is at least about 20% or at least about 30% from baseline.

For the methods described herein, any one or more aspects of health-related quality of life (such as the particular SF-36 domains described below) may be stabilized and/or improved, and the invention contemplates these methods as applied to any one or more of these aspects of health-related quality of life.

Methods of selecting individuals with SLE who are suitable for treatment according to the methods described herein are also provided. Methods of monitoring the health-related quality of life of individuals with SLE are also provided.

In the aspects and the embodiments described above, an individual of particular interest is a human.

As described herein, the sustained reduction may be effected in a number of ways, including administration of an agent (such as a dsDNA epitope) which effects the sustained reduction.

In some embodiments of each of the methods described herein, the level of anti-dsDNA antibodies is reduced in the individual with SLE. In some embodiments of each of the methods described herein, the level of circulating anti-dsDNA antibodies is reduced in the individual with SLE.

### BRIEF DESCRIPTION OF THE DRAWINGS

FIG. 1 is a graph showing the baseline SF-36 domain scores of the intent to treat (ITT) population. The scale abbreviations are as follows: “PFI” represents Physical Functioning, “ROLP” represents Role Physical, “PAIN” represents Bodily Pain, “GHP” represents General Health Perception, “VITAL” represents vitality, “SOC” represents Social Functioning, “ROLE” represents Role Emotional, and “MHI” represents Mental Health. The number of subjects varies between scales. “PBO” represents the placebo group.

FIG. 2 is a graph depicting mean changes in SF-36 domain scores of the ITT population at week 16. Scale abbreviations are as described above regarding FIG. 1. The number of subjects varies between scales.

FIG. 3 is a graph showing a comparison of the SF-36 domain scores of the active treatment group at week 16 with US norms. Scale abbreviations are as described above regarding FIG. 1. The number of subjects varies between scales.

FIG. 4 is a graph showing mean changes in SF-36 domain scores of the high affinity population. Scale abbreviations are as described above regarding FIG. 1. The number of subjects varies between scales.

FIG. 5 is a graph depicting the mean Role Emotional domain scores at each assessment point in the ITT population.

FIG. 6 provides a graph showing changes in the levels of dsDNA antibodies and C3 at each assessment point of the active treatment group in the ITT population (upper graph). The lower graph shows changes in the levels of dsDNA antibodies and C3 at each assessment point of the placebo group in the ITT population.

FIG. 7 is a graph showing the mean, pre-flare SF-36 domain scores of the portion of the ITT population having a documented renal flare. Scale abbreviations are as described above regarding FIG. 1. The number of subjects varies between scales.

FIG. 8 is a graph depicting the mean changes in SF-36 domain scores before and after a documented renal flare. Scale abbreviations are as described above regarding FIG. 1. The number of subjects varies between scales.

FIG. 9 is an illustration of frequency analysis.

FIG. 10 is a graph showing the frequency of patients with sustained reductions in anti-dsDNA antibodies. Sustained reductions in anti-dsDNA antibodies were more frequent in patients treated with LJP 394 than with placebo in the Phase II/III LJP 394 study and Phase III LJP 394 study.

FIG. 11 is a graph showing the baseline SF-36 domain scores for Phase III patients with sustained reductions of anti-dsDNA antibodies and others.

FIG. 12 is a graph showing the mean SF-36 domain score changes for Phase III patients with sustained reductions of anti-dsDNA antibodies following 48 weekly treatments, as compared to the others. Following 48 weekly treatments, HRQOL domain scores favored patients with sustained reductions.

FIG. 13 is a graph showing the mean SF-36 domain score changes for Phase III patients with sustained reductions of anti-dsDNA antibodies following 24 weekly treatments, as compared to the other Phase III patients. Following 24 weekly treatments, health-related quality of life (HRQOL) domain scores favored patients with sustained reductions.

FIG. 14 is a graph showing the mean SF-36 domain score changes for Phase III placebo patients with sustained reductions of anti-dsDNA antibodies following 24 weekly treatments, as compared to the other Phase III placebo patients. For placebo patients following 24 weekly treatments, HRQOL domain scores favored patients with sustained reductions.

FIG. 15 is a graph showing the mean SF-36 domain score changes for Phase III placebo patients with sustained reductions of anti-dsDNA antibodies following 48 weekly treatments, as compared to the other Phase III placebo patients. For placebo patients following 48 weekly treatments, HRQOL domain scores favored patients with sustained reductions.

FIG. 16 is a graph showing the mean SF-36 domain score changes for Phase III LJP 394 patients with sustained reductions of anti-dsDNA antibodies following 24 weekly treatments, as compared to the other LJP 394 patients. For drug-treated patients following 24 weekly treatments, HRQOL domain scores favored patients with sustained reductions.

FIG. 17 is a graph showing the mean SF-36 domain score changes for Phase III LJP 394 patients with sustained reductions of anti-dsDNA antibodies following 48 weekly treatments, as compared to the other LJP 394 patients. For drug-treated patients following 48 weekly treatments, HRQOL domain scores favored patients with sustained reductions.

FIG. 18 is a graph showing the mean SF-36 PCS (Physical Component Summary) and MCS (Mental Component Summary) score changes for the sustained reduction population and other population of the Phase III study following 48 weekly treatments. Following 48 weekly treatments, physical component summary score (PCS) change favored patients with sustained reductions.

FIG. 19 is a graph showing the mean SF-36 PCS and MCS score changes for the sustained reduction population and other population of the Phase III study following 24 weekly treatments. Following 24 weekly treatments, physical component summary score (PCS) change favored patients with sustained reductions.

FIG. 20 is a graph showing the mean SF-36 domain score changes for Phase II/III patients with sustained reductions of anti-dsDNA antibodies following 16 weekly treatments, as compared to the others. Following 16 weekly treatments, HRQOL domain scores appeared to also favor patients that had sustained reductions.

FIG. 21 is a graph showing the mean SF-36 PCS and MCS score changes for the sustained reduction population and other population of the Phase II/III study following 16 weekly treatments. Following 16 weekly treatments, physical component summary score (PCS) change favored patients with sustained reductions.

FIG. 22 is a graph showing the mean SF-36 domain score changes for Phase III patients with sustained reductions of anti-dsDNA antibodies and no renal flares and others with no renal flares following 24 weekly treatments. Following 24 weekly treatments and after removing renal flares, HRQOL domain scores favored patients with sustained reductions.

FIG. 23 is a graph showing the mean SF-36 domain score changes for Phase III patients with sustained reductions of anti-dsDNA antibodies and no renal flares and others with no renal flares following 48 weekly treatments. Following 48 weekly treatments and after removing renal flares, HRQOL domain scores favored patients with sustained reductions.

FIG. 24 is a graph showing mean SF 36 domain scores of Phase III patients before a renal flare. Mean domain scores were higher for LJP394 treated patients in all domains prior to a renal flare. (“Riquent™” is LJP 394.)

FIG. 25 is a graph showing mean SF 36 domain scores of Phase III patients after a renal flare. LJP394-treated patients' HRQOL scores remained better than placebo patients' after a flare. (“Riquent™” is LJP 394.)

FIG. 26 is a graph showing mean SF 36 domain score changes pre/post renal flare in Phase III patients. LJP394-treated patients appeared to have better HRQOL compared with Placebo patients in all domains except physical functioning. (“Riquent™” is LJP 394.)

FIG. 27 is a graph showing mean SF 36 domain scores of Phase II/III patients before a renal flare. Mean domain scores were higher for LJP394-treated patients in all domains except pain prior to a renal flare. (“Riquent™” is LJP 394.)

FIG. 28 is a graph showing mean SF 36 domain scores of Phase II/III patients after a renal flare. (“Riquent™” is LJP 394.) Mean domain scores were higher for LJP394-treated patients in all domains after a renal flare. (“Riquent™” is LJP 394.)

FIG. 29 is a graph showing mean SF 36 domain score changes pre/post renal flare for Phase III patients with sustained reductions of anti-dsDNA antibodies (compared to others).

FIG. 30 is a graph showing mean SF 36 PCS and MCS score changes pre/post renal flare for Phase III patients with sustained reductions of anti-dsDNA antibodies (compared to others).

FIG. 31 is a graph showing longitudinal mean SF 36 domain score changes in the patients of the Phase III study. (“Riquent™” is LJP 394.) (No substantive differences in domain scores were observed.)

FIG. 32 is a graph showing the mean PCS and MCS summary score changes for the sustained reduction population without renal flares and the others without renal flares following 24 weekly treatments in the Phase III study. Following 24 weekly treatments and after removing renal flares, physical component summary score (PCS) change favored patients with sustained reductions.

FIG. 33 is a graph showing the mean PCS and MCS summary score changes for the sustained reduction population without renal flares and the others without renal flares following 48 weekly treatments in the Phase III study. Following 48 weekly treatments and after removing renal flares, physical component summary score (PCS) change favored patients with sustained reductions.

### DETAILED DESCRIPTION OF THE INVENTION

We have discovered that administration of a dsDNA epitope that binds to SLE-associated antibodies, namely a conjugate comprising a non-immunogenic platform molecule and four double-stranded DNA epitopes, namely, LJP 394 (having four double-stranded DNA molecules with the sequence 5′-GTGTGTGTGTGTGTGTGTGT-3′(SEQ ID NO:1)), to systemic lupus erythematosis (SLE) patients effected improvement in the health-related quality of life of the patients, especially and notably among the subpopulation of patients having sustained reductions of anti-dsDNA levels and in those patients experiencing documented renal flares (with the positive effects occurring both prior to and following the occurrence of the renal flares). This result of stabilization or improvement in the health-related quality of life (HRQOL) of the patients, even in aspects of HRQOL that are dictated by emotional and mental health, and across various patient subpopulations, was surprising and unexpected.

Improvements in the health-related quality of life of individuals with SLE have not paralleled improvements in the survival rate of patients with SLE that have occurred over recent decades (Wang et al., J. Rheumatol. (2001) 28:525-532). Although the results of past studies concerning the quality of life of individuals with SLE have been somewhat inconsistent (Wang et al. (2001); Thumboo et al., J. Rheumotol. (2000) 27:1414-1420), several recent studies have indicated that there is no correlation between the quality of life of an SLE patient and either disease activity or accumulated organ damage (Hanly, Lupus (1997) 6:243-247; Abu-Shakra et al., J. Rheumatol. (1999) 26:306-309; Burckhardt, J. Rheumatol. (1993) 20:977-81; Gladman et al., Lupus (1996) 5:190-5; Gladman et al., Clin. Exp. Rheumatol. (1996) 14:305-308). Disease activity, accumulated damage, and quality of life are generally viewed in the art as three independent dimensions of the health of an individual with SLE (Hanly (1997); Abu-Shakra et al., (1999); Gladman et al., J. Rheumatol. (1996) 23:1953-1955).

The instant invention is based, in part, upon analysis of data from a clinical trial of LJP 394 referred to as the 90-05 study or Phase II/III, some accounts of which have been published as Linnik et al. (2000) Arth. Rheumat. 43(9 supplement):S241 (abstracts 1045 and 1046) and Alarcon-Segovia et al. (2000) Arth. Rheumat. 43(9 supplement):S272 (abstract 1231) and are described herein. At 16 weeks of the clinical trial, patients were found to have improved HRQOL as assessed by the Medical Outcomes 36-Item Short Form (SF-36). The general patient population of the clinical trial, as well as those subpopulations having high-affinity antibodies to the conjugate, no renal flares, or not taking high dose corticosteroids and/or cytotoxic agents (HDCC), all showed significant improvements from baseline in the Social Functioning, Role Emotional and Role Physical domains scores on the SF-36 (see Examples 6, 7, 8 and 11, below). Also, in those patients with data pre and post renal flares, those receiving LJP 394 reported stabilization or improvement in all but one of the SF-36 HRQOL domains, compared with deterioration in all domains with placebo (see Example 10, below).

The instant invention is also based in part upon the later analysis of data from a clinical trial of LJP394 referred to as the 90-09 study or Phase III study, as well as further analysis of the data from the Phase II/III study. Treatment with LJP 394 was found to be associated with a statistically significant and sustained decrease in anti-dsDNA antibodies from baseline compared with placebo (see Example 14, below). Patients with sustained reduction in anti-dsDNA antibody levels at week 24 of the study were found to have statistically significant improvements in six of the eight SF-36 HRQOL domains (physical functioning, role physical, bodily pain, general health perception, vitality, and social functioning) and apparent improvements in the other two SF-36 HRQOL domains (role emotional and mental health) relative to placebo (see Example 15, below). A statistically significant improvement in the SF-36 physical component summary score was also observed (see Example 15). Also, patients with sustained reductions in anti-dsDNA antibodies reported large improvements in HRQOL prior to and following the occurrence of renal flares (see Example 18, below). These data show that a sustained reduction in anti-dsDNA antibodies (whether effected by LJP394 or otherwise) results in an improved or stabilized HRQOL in patients and that this improvement or stabilization in the patients' HRQOL results despite the occurrence of renal flares in the patients. Data from the Phase III study also further confirmed the findings of the Phase II/III study indicating that LJP394 improved HRQOL prior to and following a renal flare (see Example 16, below). These data show that administration of LJP394 to SLE patients results in an improved or stabilized HRQOL despite the occurrence of renal flares in the patients.

Accordingly, in one aspect, the invention provides a method of stabilizing or improving the health-related quality of life in an individual with systemic lupus erythematosus, comprising administering to the individual an effective amount of a dsDNA epitope which specifically binds to an SLE-associated antibody from the individual (generally, an antibody which specifically binds to double-stranded DNA (anti-dsDNA antibody), although as is known in the art, and described herein, such antibodies may also bind single-stranded DNA and/or analogs or mimetics of double-stranded DNA), wherein the administration of the dsDNA epitope results in a stabilization of or improvement in the individual's health-related quality of life. In some embodiments, the effective amount of dsDNA epitope is administered to the individual for a period of more than about 16 weeks. In some embodiments, the dsDNA epitope is administered such that there is a sustained reduction of anti-dsDNA antibodies in the individual, wherein the sustained reduction is at least about 10% below baseline in the individual for greater than or equal to about ⅔ of all observed values prior to HDCC treatment or the last (most recent) dose of a drug (for example, LJP394) used for treatment or about ⅔ of all values measured during treatment. (The term “baseline” refers to the mean of the last two determinations of the circulating anti-dsDNA antibody level in an individual prior to initial administration of the drug. A baseline may also be established by a measurement of anti-dsDNA antibodies prior to, or upon, initial administration of the drug.) In some embodiments, the sustained reduction is maintained for more than about 16 weeks. In other embodiments, the sustained reduction is maintained for more than about 24 weeks. In some embodiments, the sustained reduction is for at least about one month, at least about two months, at least about three months, at least about 16 weeks, at least about four months, at least about five months, at least about 24 weeks, at least about 6 months, at least about 48 weeks, or at least about one year. In some embodiments, the sustained reduction is for at least about two years or longer. Ideally, treatment results in a sustained reduction for years, since SLE is a chronic disease. In further embodiments, the epitopes are attached to valency platform molecules. In some embodiments, the dsDNA epitope is administered to the individual in the form of a conjugate comprising (a) a non-immunogenic valency platform molecule and (b) two or more double-stranded DNA (dsDNA) epitopes, wherein the administration of the dsDNA epitope results in a stabilization of or improvement in the individual's health-related quality of life. In some embodiments, the dsDNA epitope comprises, consists essentially of, or consists of the double-stranded polynucleotide 5′-GTGTGTGTGTGTGTGTGTGT-3′(SEQ ID NO:1) and its complementary strand, particularly the sequence 3′-CACACACACACACACACACA-5′(SEQ ID NO:2), or one of the single-stranded polynucleotides 5′-GTGTGTGTGTGTGTGTGTGT-3′(SEQ ID NO:1) or 3′-CACACACACACACACACACA-5′(SEQ ID NO:2). In still another embodiment, the conjugate is LJP 394. In some embodiments, the stabilization or improvement in the individual's health-related quality of life occurs prior to or following a renal flare. In some embodiments, the method is a method of stabilizing the health-related quality of life of an individual with SLE. In some other embodiments, the method is a method of improving the health-related quality of life of an individual with SLE.

In another aspect, the invention provides a method of stabilizing or improving the health-related quality of life of an individual with systemic lupus erythematosus, comprising reducing the levels of circulating SLE-associated antibodies (alternatively termed anti-dsDNA antibodies) in the individual, wherein reducing the levels of circulating SLE-associated antibodies in the individual results in a stabilization of or improvement in the individual's health-related quality of life. Again, the SLE-associated antibody is generally an antibody which specifically binds to double-stranded DNA, although as is known in the art, and described herein, such antibodies may also bind single-stranded DNA and/or mimetics or analogs of double-stranded DNA. Reduction of circulating levels of antibodies can be achieved in a variety of ways, as described herein. In some embodiments, the method comprises the administering to the individual an effective amount of an agent that reduces SLE-associated antibodies in the individual. In another embodiment, the agent is administered to the individual such that there is a sustained reduction in the individual's anti-dsDNA antibody level of at least about 10% below baseline for greater than or equal to about ⅔ of all observed values prior to HDCC treatment or the last (most recent) dose of a drug (for example, LJP 394) used for treatment or about ⅔ of all values measured during treatment. In some embodiments, the sustained reduction is maintained for more than about 16 weeks. In other embodiments, the sustained reduction is maintained for more than about 24 weeks. In some embodiments, the sustained reduction is for at least about one month, at least about two months, at least about three months, at least about 16 weeks, at least about four months, at least about five months, at least about 24 weeks, at least about 6 months, at least about 48 weeks, or at least about one year. In some embodiments, the sustained reduction is for at least about two years or longer. Ideally, treatment results in a sustained reduction for years, since SLE is a chronic disease. In some embodiments, administration of the agent results in the sustained reduction of anti-dsDNA antibodies in the individual. In another embodiment, the stabilization or improvement in the individual's health-related quality of life occurs prior to or following a renal flare. In some embodiments, the method is a method of stabilizing the health-related quality of life of an individual with SLE. In some other embodiments, the method is a method of improving the health-related quality of life of an individual with SLE.

In another aspect, the invention provides a method of stabilizing or improving the health-related quality of life in an individual with SLE before, during, or following a renal flare, comprising administering to the individual an effective amount of an agent (such as, a dsDNA epitope that specifically binds to an anti-dsDNA antibody from the individual) that reduces SLE-associated antibodies in the individual. In some embodiments, the administration of the agent results in the sustained reduction of anti-dsDNA antibodies in the individual, wherein the sustained reduction is at least about 10% below baseline in the individual for greater than or equal to about ⅔ of all observed values prior to HDCC treatment or the last (most recent) dose of a drug (for example, LJP394) used for treatment or about ⅔ of all values measured during treatment. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years.

In still another aspect, the invention provides a method of stabilizing or improving the health-related quality of life of an individual with systemic lupus erythematosus, comprising effecting a sustained reduction of anti-dsDNA antibodies in the individual. In some embodiments, the sustained reduction is effected by the administration of an agent (such as, a dsDNA epitope that specifically binds to an anti-dsDNA antibody from the individual) that reduces the level of circulating anti-dsDNA antibodies in the individual. In some embodiments, the agent is administered such that there is a sustained reduction of anti-dsDNA antibodies in the individual, where the sustained reduction is at least about 10% below baseline in the individual for greater than or equal to about ⅔ of all observed values prior to HDCC treatment or the last (most recent) dose of a drug (for example, LJP394) used for treatment or about ⅔ of all values measured during treatment. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years.

In yet another aspect, the invention provides a method of stabilizing or improving the health-related quality of life in an individual with SLE prior to or following a renal flare, comprising effecting a sustained reduction of anti-dsDNA antibodies in the individual. In some embodiments, the sustained reduction is effected by the administration of an agent (such as, a dsDNA epitope that specifically binds to an anti-dsDNA antibody from the individual) that reduces the level of circulating anti-dsDNA antibodies in the individual. In some embodiments, the administration of the agent results in the sustained reduction of anti-dsDNA antibodies in the individual, wherein the sustained reduction is at least about 10% below baseline in the individual for greater than or equal to about ⅔ of all observed values prior to HDCC treatment or the last (most recent) dose of a drug (for example, LJP394) used for treatment or about ⅔ of all values measured during treatment. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years.

In still another aspect, the invention provides a method of stabilizing or improving the health-related quality of life in an individual with SLE, comprising administering to the individual an effective amount of a mimetic of double-stranded DNA, wherein the mimetic specifically binds to an SLE-associated antibody (i.e., an anti-dsDNA antibody) from the individual, and wherein the administration of the mimetic results in a stabilization of or improvement in the individual's health-related quality of life.

In an additional aspect, the invention provides a method of stabilizing or improving the health-related quality of life in an individual with SLE following a renal flare, comprising reducing the level of circulating anti-dsDNA antibodies in the individual.

In a further aspect, the invention provides a method of stabilizing or improving the health-related quality of life in an individual with systemic lupus erythematosus (SLE), comprising effecting a sustained reduction of anti-dsDNA antibodies in the individual for a period of more than about 16 weeks.

In still another aspect, the invention provides a method of stabilizing or improving the health-related quality of life in an individual with SLE following a renal flare, comprising effecting a sustained reduction of anti-dsDNA antibodies in the individual for a period of more than about 16 weeks.

In another aspect, the invention provides a method of stabilizing or improving the health-related quality of life in an individual with SLE comprising the steps of selecting an individual for receiving or continuing to receive treatment based on the individual's need for a stabilized or improved health-related quality of life, and administering a treatment to the selected individual, wherein administration of the treatment effects a sustained reduction of anti-dsDNA antibodies in the individual.

In still another aspect, the invention provides a method of stabilizing or improving the health-related quality of life in an individual having SLE comprising the steps of selecting an individual to receive or continue to receive a dsDNA epitope based on the affinity of the dsDNA epitope for an anti-dsDNA antibody in the individual, and administering the dsDNA epitope to the selected individual, wherein administration of the dsDNA epitope stabilizes or improves the health-related quality of life in an individual.

In an additional aspect, the invention provides a kit comprising a dsDNA epitope which specifically binds to an anti-dsDNA antibody from an individual with SLE, and instructions comprising a description of the administration of the dsDNA epitope to an individual to stabilize or improve the health-related quality of life in the individual.

In still another aspect, the invention provides a kit comprising a dsDNA epitope which specifically binds to an anti-dsDNA antibody from an individual with SLE, and instructions comprising a description of the selection of an individual suitable for receiving treatment by administration of the dsDNA epitope based on the low health-related quality of life of the individual.

I. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Animal Cell Culture (R. I. Freshney), ed., 1987); Methods in Enzymology (Academic Press, Inc.); Handbook of Experimental Immunology (D. M. Weir & C.C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991) and Short Protocols in Molecular Biology (Wiley and Sons, 1999). Other useful references include Harrison's Principles of Internal Medicine (McGraw Hill; J. Isseleacher et al., eds.) and Dubois' Lupus Erythematosus (5th ed.; D. J. Wallace and B. H. Hahn, eds.; Williams & Wilkins, 1997).

II. Definitions

The terms “sustained reduction in anti-dsDNA antibodies” or “sustained reduction in anti-dsDNA antibody level” or “sustained reduction” are used interchangeably herein to refer to an at least about 10% reduction in the anti-dsDNA antibody level of an individual (relative to baseline) that is maintained over an extended period of time. (The term “baseline” as used in this context generally refers to the mean of the last two determinations of the circulating anti-dsDNA antibody level in the individual prior to initial administration of the drug. A baseline may also be established by a measurement of anti-dsDNA antibodies prior to, or upon, initial administration of the drug. The about 10% reduction relative to baseline means that a value of 100 at baseline would drop at least about 10% to about 90 or an even lower value.) The about 10% or greater reduction in the anti-dsDNA antibody level of an individual having a “sustained reduction in anti-dsDNA antibodies” is generally maintained for at least two-thirds of the time period of at least about one month, at least about two months, at least about three months, at least about 16 weeks (or at least about four months), at least about five months, at least about 24 weeks (or at least about six months), or at least about 48 weeks (or at least about one year). Typically, the sustained reduction is reflected in an at least about 10% reduction in an individual's anti-dsDNA antibody level compared to baseline for greater than or equal to about two-thirds of all observed anti-dsDNA antibody level values for that individual over a given time period such as at least about one month, at least about two months, at least about three months, at least about 16 weeks (or at least about four months), at least about five months, at least about 24 weeks (or at least about six months), or at least about 48 weeks (or at least about one year). The time period over which anti-dsDNA levels are measured will be limited to those prior to treatment with HDCC or prior to the last (most recent) dose of the drug (such as LJP 394) used for treatment. It is understood that anti-dsDNA antibody levels may fluctuate (down or up) over time in individuals with sustained reduction in anti-dsDNA antibodies. In addition, in some cases (embodiments), patients with a sustained reduction of antibodies show a reduction of anti-dsDNA antibodies greater than or equal to about 20% or 30%.

A “sustained reduction” patient is a patient who has demonstrated a sustained reduction in anti-dsDNA antibodies. The subpopulation of patients who show a sustained reduction in anti-dsDNA antibodies is referred to as the “sustained reduction” subpopulation. A patient or subpopulation designated as “Other” or “other” is any patient or subpopulation of patients that did not meet criteria for sustained reduction.

When a patient's health-related quality of life or limitations on patient functioning “is used as a basis” for administration of the treatment methods described herein, or selection for the treatment methods described herein, the patient's health-related quality of life or limitations on the patient's ability to function is evaluated before and/or during treatment, and the conclusions obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. As would be well understood by one in the art, an evaluation of a patient's health-related quality of life or of the limitations on a patient's ability to function in a clinical setting is a clear indication that this parameter was used as a basis for initiating, continuing, adjusting and/or ceasing administration of the treatments described herein.

A “population” is a group of individuals with lupus.

“SLE flares” are used herein to refer to flares (i.e. acute clinical events) which occur in patients with SLE. The SLE flares may be in various major organs, including but not limited to, kidney, brain, lung, heart, liver, and skin. If the activity is in the kidneys, then the SLE flare is referred to as a “renal flare”. “Renal flares” can be identified by evaluating factors including, but not limited to, proteinuria levels, hematuria levels, and serum creatinine levels. “Reducing incidence” of renal flares in an individual with SLE means any of reducing severity (which can include reducing need for and/or amount of (e.g., exposure to) other drugs generally used for this conditions, including, for example, high dose corticosteroid and/or cyclophosphamide), duration, and/or frequency (including, for example, delaying or increasing time to renal flare as compared to not receiving treatment) of renal flare(s) in an individual.

“High dose corticosteroid and/or cyclophosphamide” or “HDCC” as used herein refers to intervention with an increased dosage of corticosteroid alone or with cyclophosphamide. High dose generally refers to corticosteroids. Such intervention generally occurs upon a flare, or acute episode. Generally, for example, the increased dosage is at least a 15 mg/day and can be greater than 20 mg/day. HDCC may be administered using standard clinical protocols. A clinician may monitor a patient and determine when HDCC treatment is needed by evaluating factors including, but not limited to, proteinuria levels, hematuria levels, and serum creatinine levels. In general, patients who experience renal flares are given HDCC treatment, although this treatment is used for other aspects of lupus.

As used herein, the singular form “a”, “an”, and “the” includes plural references unless indicated otherwise. For example, “an” antibody includes one or more antibodies.

An “epitope” is a term well-understood in the art and means any chemical moiety that exhibits specific binding to an antibody. An “epitope” can also comprise an antigen, which is a moiety or molecule that contains an epitope, and, as such, also specifically binds to antibody.

A “double-stranded DNA epitope” or “dsDNA epitope” (used interchangeably herein) is any chemical moiety which exhibits specific binding to an anti-double-stranded DNA antibody and, as such, includes molecules which comprise such epitope(s). Further discussion of double-stranded DNA epitopes suitable for use in the methods of the invention are described below. The term “dsDNA epitope” also includes mimetics of double-stranded DNA itself, which are described below. Examples of analogs or mimetics of double-stranded DNA that are encompassed by the term “dsDNA epitope” include, but are not limited to, single-stranded DNA polynucleotides that preferentially bind anti-dsDNA antibodies. Other possible mimetics of double-stranded DNA that are encompassed by the term “dsDNA epitope” include NR2 receptor epitopes such as the pentapeptide Asp/Glu-Trp-Asp/Glu-Tyr-Ser/Gly (DeGiorgio et al. (2001) Nature Medicine 7:1189-1193; Putterman and Diamond (1998) J. Exp. Med. 188:29-38; Gaynor et al. (1997) Proc. Natl. Acad. Sci. USA 94:1955-1960), so long as the NR2 receptor epitopes exhibit specific binding to anti-dsDNA antibodies from individuals having SLE.

An “NR2 receptor epitope” is any chemical moiety that exhibits specific binding to an antibody that specifically or preferentially binds N-methyl-D-aspartate (NMDA) receptor NR2a or N-methyl-D-aspartate (NMDA) receptor NR2b and, as such, includes molecules which comprise such epitope(s). Optionally, the “NR2 receptor epitope” comprises, consists essentially of, or consists of the pentapeptide sequence Asp/Glu-Trp-Asp/Glu-Tyr-Ser/Gly (DeGiorgio et al. (2001); Putterman and Diamond (1998); Gaynor et al. (1997)). Optionally, the “NR2 receptor epitope” is also a dsDNA epitope.

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a double-stranded DNA (dsDNA) epitope is an antibody that binds the dsDNA epitope with greater affinity, avidity, more readily, and/or with greater duration than it binds to non-ds DNA epitopes. It is also understood by reading this definition that, for example, an antibody (or moiety or epitope) that specifically binds to a first target may or may not specifically or preferentially bind to a second target. As such, “specific binding”, “specifically binding”, “preferentially binding”, or “preferential binding” does not necessarily require (although it can include) exclusive binding. For instance, a cross-reacting antibody that specifically binds or preferentially binds a dsDNA epitope may also specifically bind or preferentially bind the N-methyl-D-aspartate (NMDA) receptors NR2a or NR2b (DeGiorgio, et al. (2001) Nature Medicine 7:1189-1193; Putterman and Diamond, (1998) J. Exp. Med. 188:29-38; Gaynor et al., Proc. Natl. Acad. Sci. USA 94:1955-1960.). Also, an antibody that specifically binds or preferentially binds a dsDNA epitope may also specifically bind or preferentially bind a single-stranded DNA molecule.

An “anti-double-stranded DNA antibody” or “anti-dsDNA antibody” or “double-stranded DNA antibody” or “antibodies to dsDNA”, used interchangeably herein, is any antibody which specifically binds to double-stranded DNA (dsDNA). This term is used to generally refer to SLE-associated antibodies, which are antibodies whose production occurs during an SLE disease state and/or whose production is undesirable in a patient with SLE. An “anti-ds DNA antibody” can also specifically bind to a single-stranded DNA, and as such, this term includes antibodies which cross-react with single-stranded DNA, although such cross-reactivity is not required. The “ds” terminology is used in accordance with the traditional nomenclature in this field. As such, based on this definition, these antibodies could also be termed “anti-DNA” antibodies. Any antibody includes an antibody of any class, such as IgG, IgA, or IgM, and the antibody need not be of any particular class. As clearly indicated in the definition of “antibody” provided herein, an “anti-double-stranded DNA antibody” encompasses any fragment(s) that exhibits this requisite functional (i.e., specific binding to dsDNA) property, such as fragments that contain the variable region, such as Fab fragments. As discussed below, it is understood that specific binding to any anti-double-stranded DNA antibody (or functional fragment) is sufficient. Optionally, an anti-dsDNA antibody may cross-react with mimetics or analogs of the dsDNA epitope. For instance, an anti-dsDNA antibody may cross-react with a polypeptide mimetic of double-stranded DNA (e.g., the pentapeptide sequence Asp/Glu-Trp-Asp/Glu-Tyr-Ser/Gly, such as that found in the N-methyl-D-aspartate (NMDA) receptor NR2a and N-methyl-D-aspartate (NMDA) receptor NR2b (DeGiorgio, et al. (2001))).

The terms “circulating anti-double-stranded DNA antibody”, “circulating anti-dsDNA antibody”, and “circulating SLE-associated antibody”, as used herein, intends an anti-double-stranded DNA antibody which is not bound to a double-stranded DNA epitope on and/or in a biological sample, i.e., free antibody.

For purposes of this invention, “reducing” and/or “removing” SLE-associated circulating antibodies means that the level of free, or unbound, circulating SLE-associated antibodies has been reduced. Circulating SLE-associated antibodies are optionally reduced or removed by the binding of circulating SLE-associated antibodies to an administered moiety or by the induction of tolerance, including the induction of B cell anergy. In some embodiments, by binding of epitope to an antibody, the antibody is prevented from being an effector molecule, i.e., binding other targets, and is thus “reduced.” In some embodiments, “reducing” circulating antibodies includes clearance of antibody, e.g., physical removal from circulation. One way clearance is effected is clearance of a complex comprising an epitope carrier, such as an epitope-presenting valency platform molecule, and antibody by reticuloendothelial system.

An “antibody” (interchangeably used in plural form) is an immunoglobulin molecule capable of specific binding to a target, such as a carbohydrate, polynucleotide or polypeptide, through at least one antigen recognition site, located in the variable region of the immunoglobulin molecule. As used herein, the term encompasses not only intact antibodies, but also fragments thereof (such as Fab, Fab′, F(ab′)₂, Fv), single chain (ScFv), mutants thereof, fusion proteins comprising an antibody portion, humanized antibodies, and any other modified configuration of the immunoglobulin molecule that comprises an antigen recognition site of the required specificity.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. It is understood that the double stranded polynucleotide sequences described herein also include the modifications described herein. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH2) or a mixed phosphoramidate-phosphodiester oligomer. A phosphorothioate linkage can be used in place of a phosphodiester linkage. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. For purposes of this invention, a polynucleotide is generally an isolated polynucleotide of less than about 1 kb, preferably less than about 500 base pairs (bp), preferably less than about 250 bp, preferably less than about 100 bp, preferably less than about 50 bp. However, it is understood that a polynucleotide of any size or configuration could be used as long as it exhibits the requisite binding to anti dsDNA antibody from an individual. It is further understood that a different polynucleotide (for example, in terms of size and/or sequence) other than the one that is to be, was, or will be used in treatment, as long as both polynucleotides exhibit equivalent (or convertible) binding affinities to anti-dsDNA antibodies from an individual. In other words, non-identical polynucleotides may be employed with respect to affinity determination and treatment.

Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

As used herein, an “analog” or “mimetic” of an epitope means a biological or chemical compound which specifically binds to an antibody to which the epitope specifically binds. As such, a “double-stranded DNA epitope” includes mimetics of naturally-occurring double-stranded DNA. An “analog” or “mimetic” of a dsDNA epitope shares an epitope, or binding specificity, with double-stranded DNA. An analog or mimetic may be any chemical substance which exhibits the requisite binding properties, and thus may be, for example, a simple or complex organic or inorganic molecule; a polypeptide; a polynucleotide; a carbohydrate; a lipid; a lipopolysaccharide; a lipoprotein, or any combination of the above, including, but not limited to, a polynucleotide-containing polypeptide; a glycosylated polypeptide; and a glycolipid. The term “analog” encompasses the term “mimotope”, which is a term well known in the art.

An “individual” is a vertebrate, preferably a mammal, more preferably a human. Mammals include, but are not limited to, farm animals, sport animals, pets, primates, mice and rats.

“Inducing tolerance” or “inducing immunotolerance” means a reduction and/or stabilization of the extent of an immune response to an immunogen, and, as such, means immune unresponsiveness (or at least a reduction in the extent of an immune response) at the organismal level and unresponsiveness (e.g., anergy) and/or apoptosis at the cellular level. An “immune response” may be humoral and/or cellular, and may be measured using standard assays known in the art. For purposes of this invention, the immune response is generally reflected by the presence of, and/or the levels of, anti-double-stranded DNA antibodies. Quantitatively the reduction (as measured by reduction in antibody production and/or levels) is at least about 15%, preferably at least about 25%, more preferably at least about 50%, more preferably at least about 75%, more preferably at least about 90%, even more preferably at least about 95%, and most preferably 100%. It is understood that the tolerance is antigen-specific, and applies for purposes of the invention to those individuals having anti-double-stranded DNA antibodies. “Inducing tolerance” also includes slowing and/or delaying the rate of increase of antibody level.

As used herein, the term “B cell anergy” intends unresponsiveness of those B cells requiring T cell help to produce and secrete antibody and includes, without limitation, clonal deletion of immature and/or mature B cells and/or the inability of B cells to produce antibody. “Unresponsiveness” means a therapeutically effective reduction in the humoral response to an immunogen. Quantitatively the reduction (as measured by reduction in antibody production) is at least 50%, preferably at least 75% and most preferably 100%.

An “effective amount” (when used in the lupus context, or in the antibody-mediated pathology context) is an amount sufficient to effect beneficial or desired results including clinical results. An effective amount can be administered in one or more administrations. For purposes of this invention, an effective amount of an epitope, epitope-presenting carrier, or an epitope-presenting valency platform molecule described herein (or a composition comprising the same), is an amount sufficient to stabilize or improve the health-related quality of life of an individual, optionally by inducing tolerance, particularly with respect to anti-double-stranded DNA antibodies.

An “isolated” or “purified” polypeptide or polynucleotide is one that is substantially free of the materials with which it is associated in nature. By substantially free is meant at least 50%, preferably at least 70%, more preferably at least 80%, even more preferably at least 90% free of the materials with which it is associated in nature.

A “carrier”, as used herein, is a molecule which contains at least one attachment site for an epitope. One example of a carrier is a valency platform molecule.

As used herein “valency platform molecule” means a nonimmunogenic molecule containing sites which allow the attachment of a discrete number of epitopes and/or mimetic(s) of epitopes. A “valency” of a conjugate or valency platform molecule indicates the number of attachment sites per molecule for a double-stranded DNA epitope(s). Alternatively, the valency of a conjugate is the ratio (whether absolute or average) of double-stranded DNA epitope to valency platform molecule.

“Nonimmunogenic”, when used to describe the valency platform molecule, means that the valency platform molecule fails to elicit an immune response (i.e., T cell and/or B cell response), and/or fails to elicit a sufficient immune response, when it is administered by itself to an individual. The degree of acceptable immune response depends on the context in which the valency platform molecule is used, and may be empirically determined.

An epitope which is “conjugated” to a carrier or a valency platform molecule is one that is attached to the carrier or valency platform molecule by covalent and/or non-covalent interactions.

An “epitope-presenting carrier” is a carrier which contains at least one attached, or bound, epitope which is specifically bound by an antibody of interest (such as an SLE-associated antibody). Optionally, a carrier contains attached, or bound, epitopes, at least two of which are able to bind to an antibody of interest.

An “epitope-presenting valency platform molecule” is a valency platform molecule which contains attached, or bound, epitopes, at least some of which (at least two of which) are able to bind an antibody of interest.

“In conjunction with” refers to administration of one treatment modality in addition to another treatment modality, such as administration of a conjugate described herein, in addition to administration of a psychiatric medication, such as an anti-depressant, to the same individual. As such, “in conjunction with” refers to administration of one treatment modality before, during or after delivery of the other treatment modality to the individual.

An individual having “significantly impaired renal function” or “significant renal impairment” is an individual exhibiting one or more clinical signs of significant renal dysfunction, as described herein. Clinical signs of renal dysfunction include anuria, oliguria, elevated blood urea nitrogen (BUN), elevated serum creatinine, clinically significant proteinuria, hematuria, reduced creatinine clearance, and other clinical indications of renal dysfunction known in the art. As described herein, generally, an individual displays significant renal impairment if any one of more of these clinical indicia are at least above the upper limit of “normal” range, as defined in the clinical arts. In some embodiments, significant renal impairment is indicated if the value exceeds the upper limit of normal by about any of the following percentages: 10, 20, 25, 30, 50, 60, 75, 100, 125, 150, 200, 250, 275, 300, 350, 400, 450, 500. As is known in the art, with respect to at least one indicia of kidney function, such as serum creatinine, an individual can have at least about 2, 3, 5, or 10 fold or greater values compared with the upper limit of normal. Generally, an individual is determined to have, or in fact has, significant renal impairment at the onset (before the individual receives the first administration), or shortly after the onset (within about 4 weeks, preferably within about 2 weeks, preferably within about 1 week, preferably within about 5 days, preferably within about 2 days, preferably within about 1 day) upon receiving the first administration), of the therapeutic methods described herein.

When significantly impaired renal function “is used as a basis” for administration of the treatment methods described herein, or selection for the treatment methods described herein, renal function is measured before and/or during treatment, and the values obtained are used by a clinician in assessing probable or likely suitability of an individual to receive treatment(s). As would be well understood by one in the art, measurement of renal function in a clinical setting is a clear indication that this parameter was used as a basis for initiating, continuing, adjusting and/or ceasing administration of the treatments described herein.

“Affinity” of an antibody from an individual for an epitope to be used, or used, in treatment(s) described herein is a term well understood in the art and means the extent, or strength, of binding of antibody to epitope. Affinity may be measured and/or expressed in a number of ways known in the art, including, but not limited to, equilibrium dissociation constant (K_{D} or K_{d}), apparent equilibrium dissociation constant (K_{D}′ or K_{d}′), and IC₅₀ (amount needed to effect 50% inhibition in a competition assay; used interchangeably herein with “I₅₀”). It is understood that, for purposes of this invention, an affinity is an average affinity for a given population of antibodies which bind to an epitope. Values of K_{D}′ reported herein in terms of mg IgG per mL or mg/mL indicate mg Ig per mL of serum, although plasma can be used.

“Receiving treatment” includes initial treatment and/or continuing treatment. As used herein, “treatment” is an approach for obtaining beneficial or desired results, preferably including clinical results. For purposes of this invention, beneficial or desired clinical results include, but are not limited to, the stabilization or improvement of the health-related quality of life of an individual with SLE.

III. Methods of Stabilizing or Improving Health-Related Quality of Life

The invention provides a variety of methods of stabilizing and/or improving the health-related quality of life in an individual with systemic lupus erythematosus (SLE). In some embodiments of each of the following methods, the methods are methods of stabilizing the health-related quality of life of an individual, in which the administration of an agent (such as a dsDNA epitope) and/or the reduction of anti-dsDNA antibodies in the individual being treated results in a stabilization of the health-related quality of life of the individual. In other embodiments of each of the following methods, the methods are methods of improving the health-related quality of life of the individual, in which the administration of an agent (such as a dsDNA epitope) and/or the reduction of anti-dsDNA antibodies in the individual being treated results in an improvement of the health-related quality of life of the individual. A stabilization or improvement in the health-related quality of life of an individual can be in one or more different aspect of the health-related quality of life of the individual, as described elsewhere here in more detail.

In one aspect, the invention provides a method of stabilizing or improving the health-related quality of life of an individual with systemic lupus erythematosus, comprising administering to the individual an effective amount of a dsDNA epitope which specifically binds to an SLE-associated antibody (also referred to herein as an anti-dsDNA antibody) from the individual (generally, an antibody which specifically binds to double-stranded DNA, although as is known in the art, and described herein, such antibodies may also bind single-stranded DNA and/or mimetics or analogs of dsDNA), wherein the administration of the dsDNA epitope results in a stabilization of or improvement of the individual's health-related quality of life. In some embodiments, the dsDNA epitope is administered such that there is a sustained reduction of anti-dsDNA antibodies in the individual, wherein the sustained reduction is at least about 10% below baseline in the individual for greater than or equal to about ⅔ of all observed values prior to HDCC treatment or the last (most recent) dose of a drug (for example, LJP394) used for treatment or about ⅔ of all values measured during treatment. (The term “baseline” refers to the mean of the last two determinations of the circulating anti-dsDNA antibody level in an individual prior to initial administration of the drug. A baseline may also be established by a measurement of anti-dsDNA antibodies prior to, or upon, initial administration of the drug.) In some embodiments, the sustained reduction is for at least about one month, at least about two months, at least about three months, at least about 16 weeks, at least about four months, at least about five months, at least about 24 weeks, at least about 6 months, at least about 48 weeks, or at least about one year. In some embodiments, the sustained reduction is for at least about two years or longer. Ideally, treatment results in a sustained reduction for years, since SLE is a chronic disease. In another embodiment, the dsDNA epitope is administered to the individual in the form of an epitope-presenting carrier. Optionally, the epitope-presenting carrier is an epitope-presenting valency platform molecule comprising (a) a non-immunogenic valency platform molecule and (b) two or more double-stranded DNA (dsDNA) epitopes which specifically bind to an antibody from the individual which specifically binds to double-stranded DNA, wherein the administration of the conjugate results in a stabilization of or improvement in the individual's health-related quality of life. In some embodiments, the sustained reduction of anti-dsDNA antibodies in the individual is at least about 20% or at least about 30% from (below) baseline. In some embodiments, the method is a method of stabilizing the health-related quality of life of an individual with systemic lupus erythematosus. In other embodiments, the method is a method of improving the health-related quality of life of an individual with systemic lupus erythematosus.

In some embodiments, the dsDNA epitope is administered for at least about 16 weeks, for more than about 16 weeks, for at least about 24 weeks, or for at least about 72 weeks.

In another aspect, the invention provides a method of stabilizing or improving the health-related quality of life of an individual with systemic lupus erythematosus, comprising reducing the levels of circulating SLE-associated antibodies in the individual, wherein reducing the levels of circulating SLE-associated antibodies in the individual results in a stabilization of or improvement of the individual's health-related quality of life. In some embodiments, the method comprises the administering to the individual an effective amount of an agent that reduces SLE-associated antibodies in the individual. In another embodiment, the agent is administered to the individual such that there is a sustained reduction in the individual's anti-dsDNA antibody level of at least about 10% below baseline for greater than or equal to about ⅔ of all observed values prior to HDCC treatment or the last (most recent) dose of a drug (for example, LJP394) used for treatment or about ⅔ of all values measured during treatment. In some embodiments, the sustained reduction is for at least about one month, at least about two months, at least about three months, at least about 16 weeks, at least about four months, at least about five months, at least about 24 weeks, at least about 6 months, at least about 48 weeks, or at least about one year. In some embodiments, the sustained reduction is for at least about two years or longer. Ideally, treatment results in a sustained reduction for years, since SLE is a chronic disease. In some embodiments, the SLE-associated antibodies in the individual are antibodies that specifically bind double-stranded DNA and/or single-stranded DNA. In some embodiments, the SLE-associated circulating antibodies bind either strand or both strands of the double-stranded polynucleotide comprising, consisting of, or consisting essentially of a strand having the sequence 5′-GTGTGTGTGTGTGTGTGTGT-3′(SEQ ID NO:1) and the complementary strand 3′-CACACACACACACACACACA-5′(SEQ ID NO:2). Optionally, the SLE-associated antibodies bind one of the single-stranded polynucleotides 5′-GTGTGTGTGTGTGTGTGTGT-3′(SEQ ID NO:1) or 3′-CACACACACACACACACACA-5′(SEQ ID NO:2). In some embodiments, the sustained reduction of anti-dsDNA antibodies in the individual is at least about 20% or at least about 30% from baseline.

In another aspect, the invention provides a method of stabilizing or improving the health-related quality of life in an individual with SLE before, during, or following a renal flare, comprising administering to the individual an effective amount of an agent (such as, a dsDNA epitope that specifically binds to an anti-dsDNA antibody from the individual) that reduces SLE-associated antibodies in the individual. In some embodiments, the administration of the agent results in the sustained reduction of anti-dsDNA antibodies in the individual, wherein the sustained reduction is at least about 10% below baseline in the individual for greater than or equal to about ⅔ of all observed values prior to HDCC treatment or the last (most recent) dose of a drug (for example, LJP394) used for treatment or about ⅔ of all values measured during treatment. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years. In some embodiments, the sustained reduction of anti-dsDNA antibodies in the individual is at least about 20% or at least about 30% from baseline.

In another aspect, the invention provides a method of stabilizing or improving the health-related quality of life of an individual with systemic lupus erythematosus, comprising effecting a sustained reduction of anti-dsDNA antibodies in the individual. In some embodiments, the sustained reduction is effected by the administration of an agent (such as, a dsDNA epitope that specifically binds to an anti-dsDNA antibody from the individual) that reduces the level of circulating anti-dsDNA antibodies in the individual. In some embodiments, the agent is administered such that there is a sustained reduction of anti-dsDNA antibodies in the individual, where the sustained reduction is at least about 10% below baseline in the individual for greater than or equal to about ⅔ of all observed values prior to HDCC treatment or the last (most recent) dose of a drug (for example, LJP394) used for treatment or about ⅔ of all values measured during treatment. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years. In some embodiments, the sustained reduction of anti-dsDNA antibodies in the individual is at least about 20% or at least about 30% from baseline.

In another aspect, the invention provides a method of stabilizing or improving the health-related quality of life in an individual with SLE prior to or following a renal flare, comprising effecting a sustained reduction of anti-dsDNA antibodies in the individual. In some embodiments, the sustained reduction is effected by the administration of an agent (such as, a dsDNA epitope that specifically binds to an anti-dsDNA antibody from the individual) that reduces the level of circulating anti-dsDNA antibodies in the individual. In some embodiments, the administration of the agent results in the sustained reduction of anti-dsDNA antibodies in the individual, wherein the sustained reduction is at least about 10% below baseline in the individual for greater than or equal to about ⅔ of all observed values prior to HDCC treatment or the last (most recent) dose of a drug (for example, LJP394) used for treatment or about ⅔ of all values measured during treatment. In some embodiments, the sustained reduction is for at least about one month, at least about two months, at least about three months, at least about 16 weeks (at least about four months), at least about five months, at least about 24 weeks (at least about 6 months), at least about 48 weeks, or at least about one year. In some embodiments, the sustained reduction is for at least about two years or longer. Ideally, treatment results in a sustained reduction for years. In some embodiments, the sustained reduction of anti-dsDNA antibodies in the individual is at least about 20% or at least about 30% from baseline.

In an additional aspect, the invention provides a method of stabilizing or improving the health-related quality of life in an individual with SLE following a renal flare, comprising reducing the level of circulating anti-dsDNA antibodies in the individual.

In a further aspect, the invention provides a method of stabilizing or improving the health-related quality of life in an individual with systemic lupus erythematosus (SLE), comprising effecting a sustained reduction of anti-dsDNA antibodies in the individual for a period of more than about 16 weeks (in some embodiments, at least about 16 weeks). In some embodiments, the sustained reduction of anti-dsDNA antibodies occurs for at least about 24 weeks.

In still another aspect, the invention provides a method of stabilizing or improving the health-related quality of life in an individual with SLE following a renal flare, comprising effecting a sustained reduction of anti-dsDNA antibodies in the individual for a period of more than about 16 weeks (in some embodiments, at least about 16 weeks). In some embodiments, the sustained reduction of anti-dsDNA antibodies occurs for at least about 24 weeks.

In another aspect, the invention provides a method of stabilizing or improving the health-related quality of life of an individual with SLE, comprising administering to the individual for a period of more than about 16 weeks (in some embodiments, at least about 16 weeks) an effective amount of a dsDNA epitope which specifically binds to an anti-dsDNA antibody from the individual. In some embodiments, the dsDNA epitope is administered for at least about 24 weeks.

In a further aspect, the invention provides a method of stabilizing or improving the health-related quality of life of an individual with SLE following a renal flare, comprising administering to the individual for a period of more than about 16 weeks (in some embodiments, at least about 16 weeks) an effective amount of a dsDNA epitope which specifically binds to an anti-dsDNA antibody from the individual. In some embodiments, the dsDNA epitope is administered for at least about 24 weeks.

In some embodiments, the invention provides a method of stabilizing or improving the health-related quality of life of an individual with SLE, comprising reducing the levels of circulating SLE-associated antibodies that bind LJP 394, wherein reducing the levels of circulating SLE-associated antibodies results in a stabilization of or improvement in the individual's health-related quality of life.

In some embodiments, the SLE-associated antibodies are reduced by binding circulating SLE-associated antibodies and/or by inducing tolerance, including by inducing B cell anergy.

In some embodiments, the levels of circulating anti-dsDNA antibodies are reduced by at least about any one of the following amounts: 10%, 15%, 20%, 25%, 30%, 40%, 50%, 60%, 70%, 80%, 90%, or 95%. In some embodiments, the levels of circulating anti-dsDNA antibodies are reduced by at least about 20%, 25%, 30%, or 40%. In some alternative embodiments, the levels of circulating anti-dsDNA antibodies are reduced from about 10% to about 95%, from about 10% to about 70%, from about 15% to about 40%, or from about 20% to about 35%. It is understood that, for purposes of this invention, total reduction (i.e., 100%) need not be effected in order for these methods to be efficacious.

In some embodiments, the level of circulating anti-dsDNA antibodies are reduced and maintained at a sustained reduction of at least about 10% below baseline level. Sustained reduction is a reduction of at least about 10% reduction below baseline in anti-dsDNA antibody for at least majority of the times that the agent is administered. In some embodiments, the anti-dsDNA antibody levels are at least about 10% reduction below baseline for greater than or equal to about ⅔ of all observed values measured prior to HDCC or last (most recent) dose of the drug tested. Baseline anti-dsDNA antibody levels are calculated as the mean of the last two determinations prior to or upon initial administration of the drug. A baseline may also be established by a measurement of the level of anti-dsDNA antibody prior to or upon initial administration of the drug. In some embodiments, the sustained reduction is at least about 20% below baseline. In some embodiment, the sustained reduction is at least about 25% below baseline. In some embodiments, the sustained reduction is at least about 30% below baseline. In some embodiments, the sustained reduction is for at least about one month, at least about two months, at least about three months, at least about 16 weeks (at least about 4 months), at least about five months, at least about 24 weeks (at least about six months), at least about 48 weeks, at least about 1 year, or at least about two years or longer.

Methods of measuring antibody titer, either by binding or neutralizing assays, are well known in the art. Three assays are currently used in the diagnosis of SLE by measuring anti-dsDNA antibodies; these are the Farr, Crithidia, and ELISA assays. The Farr assay is considered by clinicians to be the most specific and sensitive of the three and the most useful in the prediction of flares in disease activity, especially renal flares (Smeenk et al., Rheumatol. Int. 11:101-7 (1991); ter Borg et al., Arthritis Rheum. 33:634-43 (1990)).

In some embodiments, the anti-dsDNA antibodies may be measured weekly. In other embodiments, the antibodies are measured every two weeks. In other embodiments, the antibodies are measured monthly. If requisite sustained reduction appears to be established, less frequent (or variable) measurements may be made.

In some embodiments, the levels of circulating SLE-associated antibodies are reduced by administration of an agent such as dsDNA epitope to the individual. Optionally, the dsDNA epitope is administered to the individual in the form of an epitope-presenting carrier. For instance, the published patent application, Taylor et al., United States Patent Application No. 20020103343 describes constructs comprising at least one monoclonal antibody specific for binding to complement receptor (CR1) site on primate erythrocytes, where the antibody is crosslinked to an antigen specific for a target pathogenic autoantibody, such as an anti-dsDNA antibody.

In one embodiment of the invention, the epitope-presenting carrier used in the methods is an epitope-presenting valency platform molecule, where at least one epitope of the epitope-presenting valency platform molecule specifically binds an SLE-associated antibody. Optionally, the epitope-presenting valency platform is a conjugate comprising a non-immunogenic valency platform molecule and two or more double-stranded DNA (dsDNA) epitopes. Exemplary epitope-presenting valency platforms are described below.

In another embodiment, the levels of circulating SLE-associated antibodies in a biological fluid of an individual are reduced by contacting the fluid with an epitope (optionally, in the form of an epitope-presenting carrier) ex vivo under conditions that permit the antibodies to bind epitopes on the valency platform. Suitable bodily fluids include those that can be returned to the individual, such as blood, plasma, or lymph.

Affinity adsorption apheresis is described generally in Nilsson et al. (1981) Blood 58(1):38-44; Christie et al. (1993) Transfusion 33:234-242; Richter et al. (1997) ASAIO J. 43(1):53-59; Suzuki et al. (1994) Autoimmunity 19: 105-112; U.S. Pat. No. 5,733,254; Richter et al. (1993) Metabol. Clin. Exp. 42:888-894; Richter et al. (1997) ASAIO J. 43(1):53-59; and Wallukat et al. (1996) Int'l J. Card. 54:191-195.

Accordingly, the invention includes methods of reducing levels of SLE-associated antibodies in an individual, comprising treating the individual's blood (including any component thereof which contains antibody) extracoporeally (i.e., outside the body or ex vivo) with an epitope (optionally in the form of an epitope-presenting carrier) under conditions that permit the antibodies to bind the epitope; removing antibody-epitope complexes, if any; and returning the blood to the individual.

In the methods of the invention, the bodily fluid is removed from the individual for extracorporeal binding to an epitope, such as an epitope-presenting valency platform molecule, of this invention. For example, apparatuses and methods for removing blood and separating it into its constituent components are known in the art (see, e.g., U.S. Pat. Nos. 4,086,924; 4,223,672). The blood or portions thereof are then exposed to the valency platform molecule. The valency platform molecule neutralizes (i.e., binds) the unwanted antibody, and the blood components are then returned to the individual.

In a preferred technique, the antibody-valency platform molecule complex is removed before the fluid is returned to the individual. This may be done, for example, by using a valency platform molecule attached to a solid phase, or by using a soluble valency platform molecule and selectively removing the complex from the treated solution.

To create a solid phase, the valency platform molecule is adapted to render it insoluble. For example, an additional linkage can be added to the valency platform molecule. The linkage is then used to attach the platform to an insoluble structure, such as a polystyrene or polyethylene bead, a polycellulose membrane, or other desirable structure. Commercially available matrices include agarose (a neutral linear polysaccharide generally composed of D-galactose and altered 3,6-anhydrogalactose residues, for example Sepharose™, Pharmacia), activated gels, nitrocellulose, borosilicate, glass fiber filters, silica, polyvinylchloride, polystyrene, and diazotized paper. Methods for preparing peptide-peptide conjugates are described in Hermanson, G. T., “Bioconjugate Techniques”, Academic Press: New York, 1996; and in “Chemistry of Protein Conjugation and Cross-linking” by S. S. Wong, CRC Press, 1993. The biological fluid to be treated is contacted with the solid phase, and antibodies in the fluid complex to the solid phase. The supernatant fluid can then be removed from the solid phase for return to the individual. In some instances, the solid phase can also be cleared of antibody for repeat use by using a suitable wash, providing both the epitope and the valency platform molecule is resistant to the washing solution. Suitable washing solutions may include 0.1 M glycine buffer, pH 2.4, dilute acetic acid, or 1 M KSCN buffered to ˜pH 7.

If the valency platform molecule is not part of a solid phase, then the antibody-carrier complex can be removed from the fluid by any other appropriate method, including but not limited to microfiltration, antibody capture, or precipitation. Solutions suitable to cause precipitation of the complex depend on the solubility of the complex, and may include ammonium sulfate or polyethylene glycol. If the fluid is to be returned to the individual, then the precipitating solution should be chosen so that any that remains in the fluid does not cause an adverse reaction in the individual.

It is understood that the in vivo and ex vivo methods for reducing circulating SLE-associated antibodies described herein may be used in conjunction with each other.

Devices which can be used for reducing the level of antibody in a biological fluid using an epitope (or epitope-presenting carrier) described herein include a flow system, comprising the following elements: a) a port that permits biological fluid to flow into the device; b) a chamber in which the fluid is permitted to contact the epitope-bound valency platform molecule (optionally in a solid phase); c) a port that permits the treated fluid to flow out of the device. Such devices can be designed as continuous flow systems, and as systems that permit the treatment of a single sample from an individual for purposes of analysis or readministration at a subsequent time.

In still another aspect, the invention provides a method of stabilizing or improving the health-related quality of life in an individual with SLE, comprising administering to the individual an effective amount of an epitope that specifically binds to an SLE-associated antibody from the individual that, and wherein the administration of the epitope results in a stabilization of or improvement in the individual's health-related quality of life.

With respect to the methods described herein that relate to stabilizing, improving, assessing, or monitoring the health-related quality of life of an individual with SLE, health-related quality of life encompasses a variety of aspects clinically recognized to contribute to an individual's sense of well being. These aspects may be distinct from other aspects of an individual's health, such as disease activity and cumulative physical damage. The invention includes any one or more aspects of health-related quality of life.

Accordingly, in one embodiment of the invention, the stabilization of or improvement in the individual's health-related quality of life comprises stabilization of or improvement in one or more aspects of health-related quality of life selected from the group consisting of limitations in physical activities because of health problems, limitations in social functioning because of physical or emotional problems, limitations in work or other usual activities because of physical health problems, bodily pain, general mental health, limitations in work or other usual activities because of emotional problems, vitality, and general health perception. In another embodiment, the one or more aspects of health-related quality of life is selected from the group consisting of limitations in social activities because of physical or emotional problems, general mental health, limitations in work or other usual activities because of emotional problems, and vitality. In another embodiment, the one or more aspects of health-related quality of life are selected from the group consisting of limitations in physical activities because of health problems, limitations in social functioning because of physical or emotional problems, and limitations in work and other usual activities because of emotional problems.

In some embodiments, the stabilization or improvement of the individual's health-related quality of life comprises the prevention or reduction of limitations on the individual's ability to function due to mental and/or emotional problems. In another embodiment, the stabilization or improvement in an individual's ability to function comprises the prevention or reduction of limitations on the individual's ability to work or participate in other usual activities because of mental and/or emotional problems. In an alternative embodiment, the stabilization or improvement of an individual's ability to function comprises the prevention or reduction of limitations on the individual's social functioning due to physical and/or emotional problems. Optionally, the stabilization or improvement of an individual's ability to function comprises the prevention or reduction of limitations on the individual's social functioning due to mental and/or emotional problems.

In still another embodiment, the stabilization or improvement in an individual's health-related quality of life comprises both the prevention or reduction of limitations on the individual's ability to work or participate in other usual activities because of emotional problems and the prevention or reduction of limitations on the individual's social functioning due to physical and/or emotional problems.

In another embodiment, the stabilization or improvement in an individual's health-related quality of life comprises the prevention or reduction of limitations in physical activities because of health problems, limitations in work or other usual activities because of physical health problems, vitality, and bodily pain.

In another embodiment, the stabilization or improvement in an individual's health-related quality of life comprises the prevention or reduction of limitations in physical activities because of health problems, limitations in social functioning because of physical or emotional problems, limitations in work or other usual activities because of physical health problems, bodily pain, vitality, and general health perception.

Optionally, the methods of the invention further comprise the additional step of assessing the stabilization of or improvement in the health-related quality of life of the individual. In some embodiments the assessment is done before treatment (and may be used as a basis for treatment). In other embodiments, the assessment is done during treatment. In still further embodiments the assessment is done following treatment. The assessment may be qualitative or quantitative.

In some embodiments, the current status of a patient's health-related quality of life can be determined as part of an examination by a physician. The questioning may be informal, and a skilled physician will be able to ascertain to a reasonable degree the qualitative nature of a patient's health-related quality of life.

Alternatively, the stabilization or improvement in health-related quality of life is assessed using one or more instruments selected from the group consisting of the Medical Outcome Study Short Form 36 (SF-36), EuroQOL (EQ-5D) (Wang et al. (2001)), QOL scale (QOLS) (Abu-Shakra et al. (1999); Burckhardt et al. (1993)), Medical Outcome Study Short Form (SF-20) (Hanly (1997)); Stanford Health Assessment Questionnaire (HAQ) (Gilboe et al., J. Rheumatol. (1999) 26:1694-1700), Functional Ability Index, Fatigue Severity Scale, Disability Days Measure, and Center for Epidemiological Studies—Depression. Any one or more of these may be used.

The Medical Outcomes Study 36-Item Short Form (SF-36) is a widely validated generic patient questionnaire shown to be sensitive to change in a variety of chronic diseases: hypertension and cardiovascular disease, diabetes, pulmonary disease, low back pain, rheumatoid arthritis (RA) and osteoarthritis (Ware J E, et al. (1992) Medical Care 30:473-483).

The SF-36 consists of 36 questions representing eight important health concepts, each of which is scored on an individual “domain” scale: Physical Functioning, Role-Physical, Bodily Pain, General Health, Vitality, Social Functioning, Role-Emotional and Mental Health (Ware et al. (1992) Medical Care 30: 473-483). The “Physical Functioning” scale measures to what degree a patient is limited in his or her ability to perform a wide variety of physical activities due to health. The “Role Physical” scale measures the degree to which a patient is limited in performing work or other usual daily activities as a result of his or her physical health. The “Role Emotional” scale measures the degree to which a patient is limited in performing work or other usual daily activities as a result of his or her mental or emotional problems. The “Bodily Pain” scale reflects the frequency and extent of pain or discomfort and also the extent to which that pain interferes with normal activities. The “Social Functioning” domain scale indicates the degree and frequency of interference with normal social activities due to physical and/or emotional problems. The “General Mental Health” scale reflects the patient's position with respect to four major mental health dimensions—anxiety, depression, loss of behavioral or emotional control, and psychological well-being. The “Vitality” scale is a measure of energy level and fatigue. The final domain scale, “General Health Perception”, reflects the patient's own belief about the status of his or her personal health. Additional information on the nature of the SF-36 domains can be found in Ware et al. (1992).

In some embodiments of each of the methods described herein, the stabilization or improvement in the individual's health-related quality of life is detectable by the Medical Outcome Survey Short Form 36 (SF-36), wherein the stabilization or improvement is reflected in one or more domains of health-related quality of life selected from the group consisting of physical functioning, role physical, bodily pain, general health perception, vitality, social functioning, role emotional, and mental health. In alternative embodiments, the stabilization or improvement in the individual's health-related quality of life is detectable by the Medical Outcome Survey Short Form 36 (SF-36), wherein the stabilization or improvement is reflected in one or more domains of health-related quality of life selected from the group consisting of physical functioning, role physical, bodily pain, general health perception, vitality, social functioning, and mental health. In other alternative embodiments, the stabilization or improvement is reflected in one or more domains of health-related quality of life selected from the group consisting of physical functioning, role physical, bodily pain, general health perception, vitality, and social functioning. In still other embodiments of each of the methods described herein, the stabilization or improvement is reflected in one or more domains of health-related quality of life selected from the group consisting of role physical, bodily pain, general health perception, and vitality. In still other embodiments of each of the methods described herein, the stabilization or improvement is reflected in one or more domains of health-related quality of life selected from the group consisting of bodily pain, general health perception, and vitality.

In addition to the individual SF-36 domain scales, composite scores may also be used to characterize a person's HRQOL. Published algorithms are available for calculating Physical Component Summary Score (PCS) and Mental Component Summary Score (MCS) (see Ware et al. (1994) Physical and Mental Component Summary Scales: A User's Manual, Boston, Mass.).

It has been translated into many languages and normative data is available across a wide number of cultures. In 1995, a Systemic Lupus International Cooperating Clinics (SLICC) workshop concluded the SF-36 to be the instrument of choice in evaluating health-related quality of life in SLE (Gladman D D, et al. (1995) J Rheumatol 23:1853-5). The SF-36 is a particularly useful instrument for assessing health-related quality of life in SLE patients as it includes questions relating to fatigue and depression.

Data from longitudinal observational studies have shown that deteriorating composite SF-36 scores reflect significant decrements in multiple domains of health-related quality of life in patients with active SLE, which correlate with increases in disease activity and damage (Dobkin P L, et al. (1998) Arthritis Care Res. 11:23-31, Gladman D D (1996) Clin Exp Rheumatol 14:305-8, Stoll T, et al. (1997) J Rheum 24:309-13, Stoll T, et al. (1997) J Rheum 24: 1608-14, Burckhardt C S, et al. (1993) J Rheumatol 20:977-81, Hanly J G (1997) Lupus 6:243-7, Fortin P R, et al. (1998) Lupus 7:101-7, Abu-Shakra M, et al. (1999) J Rheum 26:306-9, Thumboo J, et al. (1999) J Rheumatol 26:97-102, Rood M J, et al. (2000) J Rheum 27:2057-9, Wang C, et al. (2001) J Rheumatol 28:525-32). In 1998, the international consensus Outcome Measures in Rheumatology Clinical Trials (OMERACT) meeting recommended that four domains be regularly assessed in randomized controlled trials in SLE: disease activity, damage, health-related quality of life, and adverse events (Smolen J, et al. (1999) J Rheumatol 26:2:504-507). Assessment of health-related quality of life in patients with active disease is important to health care decision-makers when evaluating treatment options and allocating resources since decrements in multiple domains mirror patients' difficulty to work and engage in regular daily activities.

Improvement or stabilization of an individual's HRQOL is optionally indicated after treatment has begun by a maintenance or increase in an individual's score on one of the eight domain scales of the SF-36 or on one of the summary scores MCS or PCS, as compared to the score at baseline.

Regardless of what instrument is used to assess the stabilization or improvement in health-related quality of life, the assessment may optionally involve an assessment of a patient's health-related quality of life either immediately before or a shortly before administration of an epitope, such as an epitope-presenting valency platform molecule, to the patient or before the reduction in the levels of circulating SLE-associated antibodies is effected. The assessment may optionally occur the day treatment is initiated or optionally about 1 day, about 1 week, about 4 weeks before treatment begins. The assessment may also involve an assessment of the health-related quality of life at a point following the initial administration of the epitope or following the beginning of the reduction in the levels of circulating antibodies. For instance, an assessment at this later point may take place about 1 week, about 4 weeks, about 8 weeks, about 12 weeks, about 16 weeks, about 24 weeks, about 36 weeks, about 48 weeks, about 60 weeks, or about 72 weeks following initiation of treatment.

The methods of the invention are optionally practiced during the administration of a clinical trial. In some embodiments, the health-related quality of life of a subject of an SLE clinical trial is assessed, the subject is then administered a dsDNA epitope (e.g., double-stranded DNA, or an analog or mimetic thereof), and at some later point, the health-related quality of life of the subject is assessed again and the results of the second assessment are compared to the results of the earlier assessment. Optionally, when a drug or drug candidate is administered to a population of individuals having SLE, such as in a clinical trial, then the ability of the drug or drug candidate to improve or stabilize the HRQOL in an individual is optionally evidenced by changes in the mean domain scores or the summary scores of the population or a subpopulation of the population as compared to the scores at baseline. Alternatively, the changes in the scores of population or subpopulation are compared to changes in the mean domain score or summary scores of an equivalent population or subpopulation that has been administered placebo instead of the drug or drug candidate.

The evaluation of the effect of the ds-DNA epitope (e.g., double-stranded DNA, or an analog or mimetic thereof) on the health-related quality of life of patients may be a primary or a secondary objective of the clinical trial. For instance, the clinical trial may be run primarily to assess whether a particular drug candidate is suitable for use in treating patient's suffering from low HRQOL. Alternatively, the clinical trial is run primarily to assess a drug candidate's ability to decrease the time to renal flare or to address another physical aspect of SLE, and only secondarily is the drug candidate's effect on HRQOL assessed.

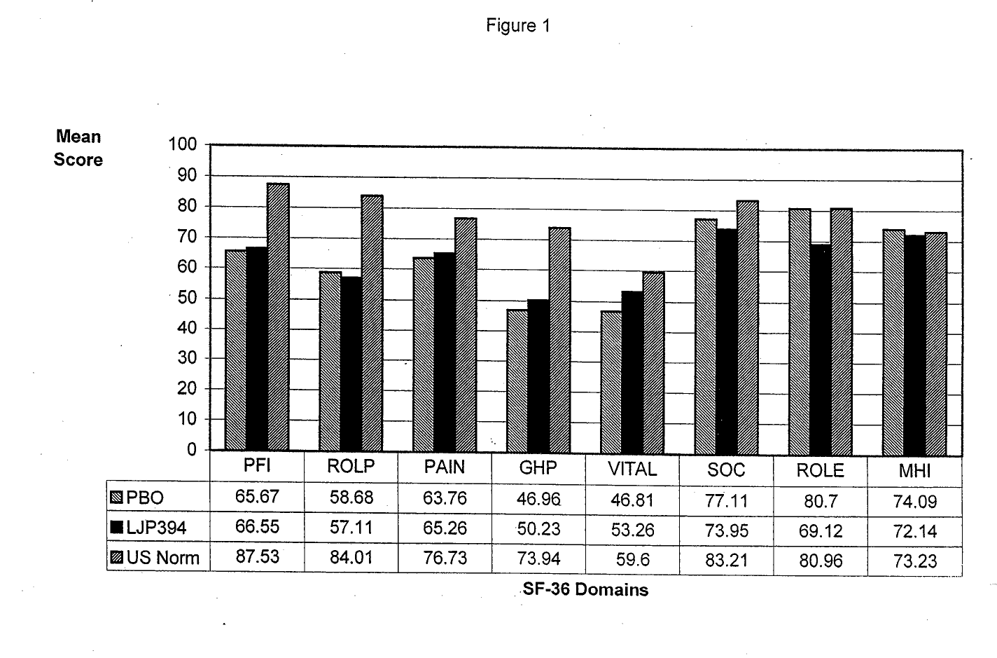

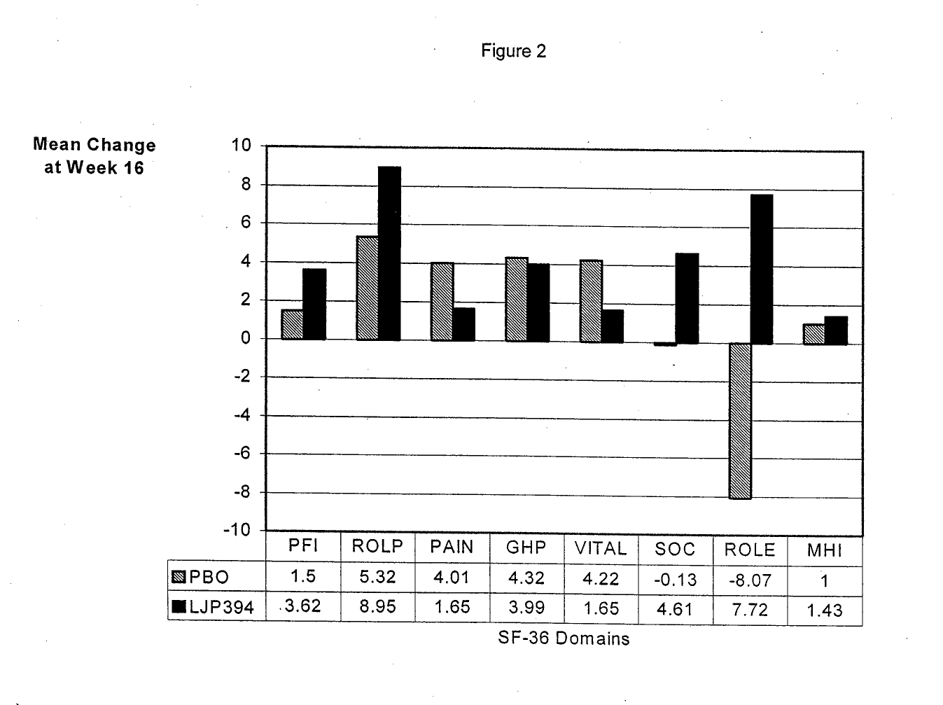

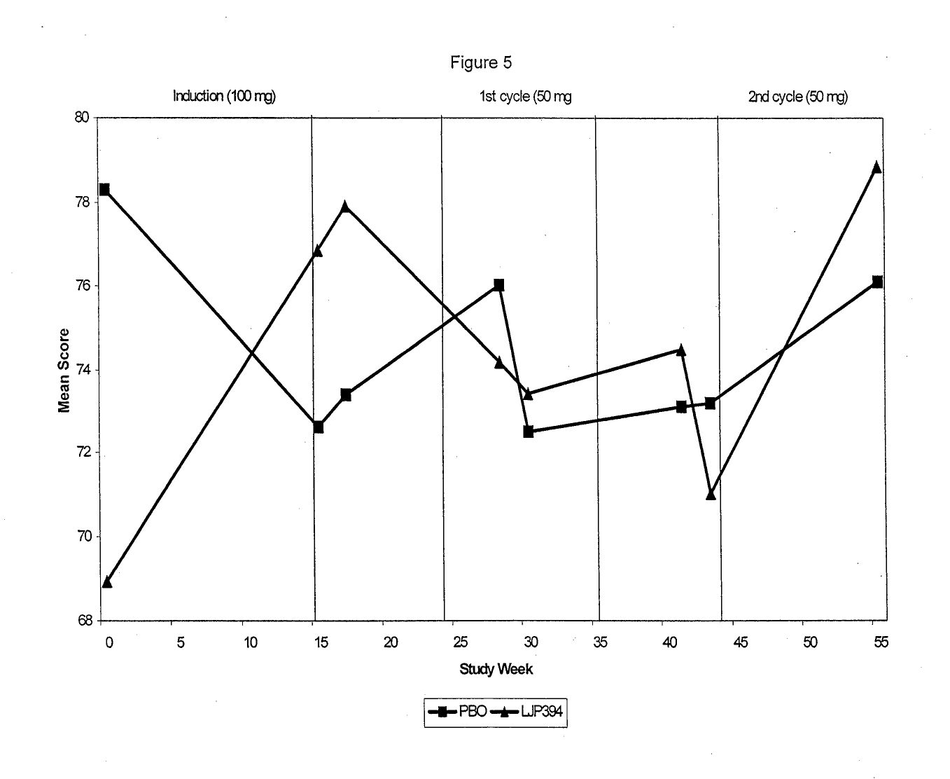

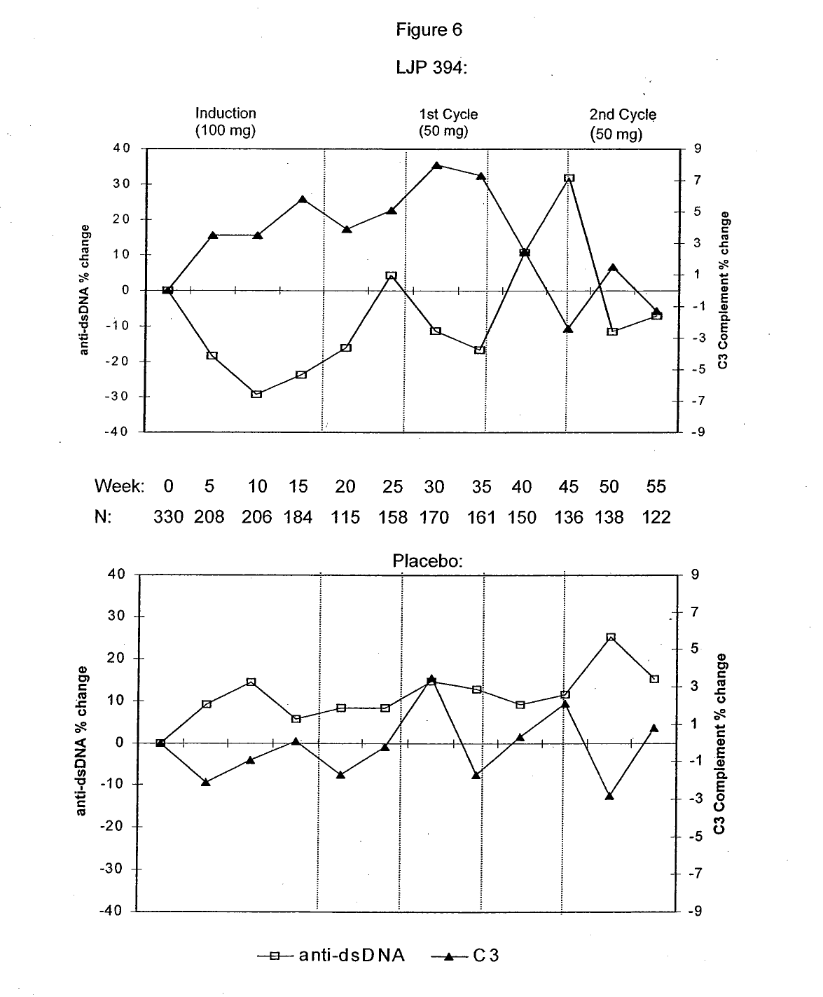

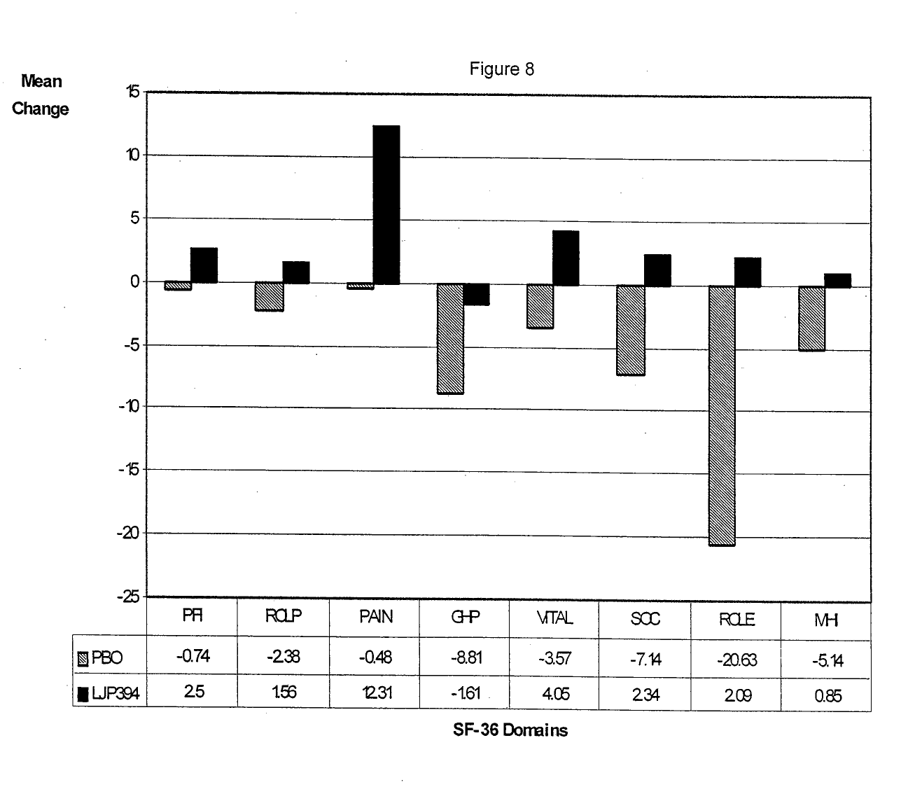

An example of a clinical trial in which a drug candidate's efficacy is evaluated for its effect on the health-related quality of life of patients is described in the Examples 1-11, below. In the clinical trial, an dsDNA epitope-presenting valency platform molecule, LJP394, was administered to SLE patients (Examples 1-2, below). The SF-36 questionnaire was completed by subjects of the clinical trial at baseline and also at various stages of the trial and the results were analyzed (Examples 3-4, below). At baseline all domains of SF-36 were decreased compared with age and gender matched US norms (Example 5, below, and FIG. 1). Mean changes from baseline in the SF-36 domain scores of the intent to treat population, the high affinity (HA) population, and other subgroups were also determined (Examples 6-8 and Example 11, below, and FIGS. 2-4). Longitudinal changes in SF-36 scores were also tracked and compared to changes in dsDNA antibodies and C3 (Example 9, below, and FIGS. 5 and 6). Mean changes in SF-36 domain scores from before the documented occurrence of renal flares to after the documented renal flares were also analyzed (Example 10, below, and FIG. 8).

As Example 6, below, indicates, the administration of 100 mg weekly of LJP 394 versus placebo to SLE patients for 16 weeks resulted in significant mean changes in the Role Emotional Scores (+7.72 points versus −8.07 points), Social Functioning scores (+4.61 points versus −0.13 points), and Role Physical scores (+8.95 versus −5.32 points). Changes were similar in the high affinity (HA) population (Example 7, below). Excluding patients with renal flares and/or receiving high dose corticosteroids and/or cytotoxic agents did not alter results (Examples 8 and 11, below). In patients with data pre- and post-renal flares, those receiving LJP 394 reported stabilization or improvement in all but one domain compared with deterioration in all with placebo (Example 10, below). Role Emotional scores differed by 20.2 points for LJP 394 versus placebo; differences in change scores favored active treatment in all domains, ranging from 9.3 to 17.16 (Example 10, below). Results were similar when patients receiving HDCC prior to renal flare were excluded from the analysis.

The Phase 3 trial enrolled 298 lupus patients with high-affinity antibodies to LJP 394 who were treated for up to 22 months with either LJP 394 or placebo. Patients completed the SF-36 assessment at entry, followed by three additional time points in the trial depending on how long they participated. Patients who had a renal flare completed the assessment once the renal flare was confirmed. (The Phase ⅔ trial enrolled 198 lupus patients with high-affinity antibodies who were treated for up to 18 months.)

In the Phase III study at 48 weeks (about 12 months), all eight SF 36 domain scores favored patients with sustained reductions in antibodies to dsDNA (n=80), when compared with patients that did not have sustained reductions (n=110) and six of the eight domain scores were statistically significant (Example 15, below). Similar results were seen at 24 weeks (about six months) (Example 15, below). The physical functioning component summary score was significant at both six and 12 months (Example 15). Twice as many LJP394-treated patients had sustained reductions as placebo-treated patients (Example 14). (Because of the design of the trial, the six and twelve month time points, rather than the 22 month time point, included the largest number of patients for analysis in the Phase III study.)

In the Phase II/III study, at 16 weeks, all eight SF 36 domains also favored patients with sustained reductions in antibodies to dsDNA (n=118), when compared with patients that did not have sustained reductions (n=58) (Example 15). Furthermore, two of the eight domain scores reached statistical significance even by this early time point when compared with patients that did not experience sustained reductions. Four times as many LJP394-treated patients had sustained reductions as placebo-treated patients (Example 14). (Because of the dose-regimen of this trial, where patients received less drug after 16 weeks, the 16-week time point was considered the most relevant for analysis in the Phase II study.)

Furthermore, as seen in the previous Phase II/III trial, data from the Phase III trial also demonstrate that LJP394-treated patients reported improved HRQOL following a renal flare compared with placebo-treated patients (Example 16). Following a renal flare, seven of eight domains of the SF-36 survey were more favorable for LJP394-treated patients compared with placebo-treated patients. Similar results were seen in the Phase II/III trial where all eight domains were more favorable for LJP394-treated patients following a renal flare compared with placebo-treated patients. In both trials, prior to a renal flare, LJP394-treated patients had generally improved HRQOL compared with placebo-treated patients. (For this analysis, HRQOL was measured after a renal flare and compared with the most recent score before the renal flare. These differences in HRQOL scores were observed despite immunosuppressive therapy given for renal flare. The differences were not statistically significant due to the small number of total renal flares in each trial.)

The longitudinal differences in HRQOL between the drug-treated and placebo-treated groups (not limited to the sustained reduction subpopulations) were not significantly different at various time points during the trial and mirror the renal flare results reported for the Phase III trial (Example 17). The lack of significance may have been due to changes in medical practice during the trial and a loss of susceptible patients as discussed in Example 17.

Improvement or stabilization in a treatment group may be statistically significant compared with placebo. However, even if the improvement or stabilization is statistically significant, the improvement or stabilization may not necessarily be clinically meaningful or readily understood. Recent efforts designed to develop consensus regarding outcome measures in randomized controlled trials have included discussion of “minimum clinically important differences” (MCID). Such differences should reflect degrees of improvement that would be perceptible to patients, on an individual basis, and would be considered clinically meaningful to them. Improvements of 33 to 36% over baseline (or 18% greater than placebo) are thought to be clinically important (Goldsmith C, et al. (1993) J Rheumatol 20:561-565, Wells G A, et al. (1993) J Rheumatol 20:557-60). Although these definitions are relevant only on an individual patient basis, when mean and median changes within a treatment group well exceed such a value it can be assumed that the majority of the group will have attained clinically important improvements.

Data from randomized controlled trials in a variety of disease states have suggested minimum clinically important differences of 5 to 10 points in individual domain scales of the SF-36 and 2.5 to 5 points for the composite physical component (PCS) and mental component summary (MCS) scores, as they reflect a magnitude of change perceptible to patients (Kavanaugh A, et al. (2000) Arth Rheum 3:147, Kosinski M, et al. (2000) Arth Rheum 43:1478-87, Kujawski S C, et al. (2000) Arth Rheum 43:S140, Samsa G, et al. (1999) Pharmacoeconomics 15:141-155). Kujawski, Thumboo, Ehrich, Stucki, and others have reported that changes of this magnitude are associated with meaningful clinical improvements and can be considered to represent MCID in rheumatoid arthritis (RA), SLE and osteoarthritis (OA) (Tugwell P, et al. (2000) Arth Rheum 43:506-14, Strand V, et al. (2001) Correlation of HAQ with SF-36, Arth Rheum 44:S187, Strand V, et al. (2001) Use of Minimum Clinically Important Differences in Evaluating Patient Responses to Treatment of RA, Arth Rheum 44:S187, Zhao S Z, et al. (1999) Pharmacotherapy 19:1269-1278, Ehrich E W, et al. (1998) Arth Rheum, 41:S221, Ehrich E W, et al. (2000) J Rheumatol 27:2635-41, Angst F, et al. (2001) Arth Care Res 45:384-391, Samsa G, et al. (1999) Pharmacoeconomics 15:141-155).

In recent longitudinal and randomized controlled trials in RA, OA and SLE, as well as chronic cardiovascular and pulmonary conditions, changes in a variety of patient reported outcome measures, including global assessments of disease activity/severity, pain, and physical function, have correlated with observed changes in SF-36 domains, as well as PCS and MCS summary scores. Wyrwich et al. compared the standard error of measurement (SEM) in SF-36 domains to MCID differences in the Chronic Heart Failure Questionnaire in one randomized clinical trial, and the Chronic Respiratory Disease Questionnaire in another (Wyrwich K W, et al. (1999) Medical Care 37:469-78, Wyrwich K W, et al. (1999) J Clin Epidemiol 52:861-73). In both studies, a value of one SEM in change scores for SF-36 domains closely approximated MCIDs for disease specific questionnaire components. The SEMs for SF-36 domain change scores ranged from 7.88 to 15.26 in the first comparison and 7.65 to 14.15 in the second. Kosinski and Ware compared changes in Health Assessment Questionnaire disability index [HAQ DI] and SF-36 domains and summary scores with patient global assessments and pain in two randomized controlled trials comparing COX-2 selective agents to traditional nonsteroidal anti-inflammatory drugs (NSAIDs) in active RA (Kosinski M, et al. (2000) Arth Rheum 43:1478-87). Mean changes in SF-36 domain scores corresponding to one level of improvement in patient global assessment or pain ranged from 4.2-21.0, and 1.9-10.8; 4.4 and 3.0 for PCS and 4.7 and 2.2 for MCS summary scores. Using the same technique to evaluate improvement in HAQ DI yielded good agreement (−0.24 to −0.22) with previously published values for MCID of −0.22.

In some embodiments, the invention provides methods where stabilization or improvement in the individual's health-related quality of life is evidenced by a maintained or increased Mental Component Summary (MCS) score on the Medical Outcome Survey Short Form 36 (SF-36). The improvement in the MCS score is optionally a clinically important increase of at least about 2.5 points over baseline. In an alternative embodiment, the improvement in the MCS score is at least about 33% over baseline or at least about 36% over baseline.

In some embodiments, the invention provides methods where stabilization or improvement in the individual's health-related quality of life is evidenced by a maintained or increased Physical Component Summary (PCS) score on the Medical Outcome Survey Short Form 36 (SF-36). The improvement in the PCS score is optionally a clinically important increase of at least about 2.5 points over baseline. In an alternative embodiment, the improvement in the PCS score is at least about 33% over baseline or at least about 36% over baseline.

In another embodiment, the stabilization or improvement in the individual's ability to function is evidenced by a maintained or increased Role Emotional domain score on the Medical Outcome Survey Short Form 36 (SF-36). The increase in the Role Emotional domain score may optionally be a clinically important increase of at least about 5 points, at least about 10 points, at least about 15 points, or at least about 20 points as compared to a baseline measurement. In an alternative embodiment, the Role Emotional domain score is at least about 33% over baseline or at least about 36% over baseline. In some embodiments, the increase in the Role Emotional domain score as compared to baseline is measured at 16 weeks following the initiation of treatment.

In another embodiment, the stabilization or improvement in the individual's ability to function is evidenced by a maintained or increased Social Functioning domain score on the Medical Outcome Survey Short Form 36 (SF-36). The increase in the Social Functioning domain score is optionally a clinically important increase of at least about 5 points, at least about 10 points, at least about 15 points, or at least about 20 points as compared to a baseline measurement. In an alternative embodiment, the Social Functioning domain score is at least about 33% over baseline or at least about 36% over baseline. In some embodiments, the increase in the Social Functioning domain score as compared to baseline is measured at 16 weeks following initiation of treatment.

In another embodiment, the stabilization or improvement in an individual's health related quality of life is evidenced by a maintained or increased Role Physical domain score on the Medical Outcome Survey Short Form 36 (SF-36). The increase in the Role Physical domain score may optionally be a clinically important increase of at least about 5 points, at least about 10 points, at least about 15 points, or at least about 20 points as compared to a baseline measurement. In an alternative embodiment, the Role Physical domain score is at least about 33% over baseline or at least about 36% over baseline. In some embodiments, the increase in the Role Physical domain score as compared to baseline is measured at 16 weeks following the initiation of treatment.

In another embodiment, the stabilization or improvement in an individual's health related quality of life is evidenced by a maintained or increased Physical Functioning domain score on the Medical Outcome Survey Short Form 36 (SF-36). The increase in the Physical Functioning domain score may optionally be a clinically important increase of at least about 5 points, at least about 10 points, at least about 15 points, or at least about 20 points as compared to a baseline measurement. In an alternative embodiment, the Physical Functioning domain score is at least about 33% over baseline or at least about 36% over baseline. In some embodiments, the increase in the Physical Functioning domain score as compared to baseline is measured at 16 weeks following the initiation of treatment.

In another embodiment, the stabilization or improvement in an individual's health related quality of life is evidenced by a maintained or increased Bodily Pain domain score on the Medical Outcome Survey Short Form 36 (SF-36). The increase in the Bodily Pain domain score may optionally be a clinically important increase of at least about 5 points, at least about 10 points, at least about 15 points, or at least about 20 points as compared to a baseline measurement. In an alternative embodiment, the Bodily Pain domain score is at least about 33% over baseline or at least about 36% over baseline. In some embodiments, the increase in the Bodily Pain domain score as compared to baseline is measured at 16 weeks following the initiation of treatment.

In another embodiment, the stabilization or improvement in an individual's health related quality of life is evidenced by a maintained or increased General Health Perception domain score on the Medical Outcome Survey Short Form 36 (SF-36). The increase in the General Health Perception domain score may optionally be a clinically important increase of at least about 5 points, at least about 10 points, at least about 15 points, or at least about 20 points as compared to a baseline measurement. In an alternative embodiment, the General Health Perception domain score is at least about 33% over baseline or at least about 36% over baseline. In some embodiments, the increase in the General Health Perception domain score as compared to baseline is measured at 16 weeks following the initiation of treatment.

In another embodiment, the stabilization or improvement in an individual's health related quality of life is evidenced by a maintained or increased Vitality domain score on the Medical Outcome Survey Short Form 36 (SF-36). The increase in the Vitality domain score may optionally be a clinically important increase of at least about 5 points, at least about 10 points, at least about 15 points, or at least about 20 points as compared to a baseline measurement. In an alternative embodiment, the Vitality domain score is at least about 33% over baseline or at least about 36% over baseline. In some embodiments, the increase in the Vitality domain score as compared to baseline is measured at 16 weeks following the initiation of treatment.

In another embodiment, the stabilization or improvement in an individual's health related quality of life is evidenced by a maintained or increased Mental Health domain score on the Medical Outcome Survey Short Form 36 (SF-36). The increase in the Mental Health domain score may optionally be a clinically important increase of at least about 5 points, at least about 10 points, at least about 15 points, or at least about 20 points as compared to a baseline measurement. In an alternative embodiment, the Mental Health domain score is at least about 33% over baseline or at least about 36% over baseline. In some embodiments, the increase in the Mental Health domain score as compared to baseline is measured at 16 weeks following the initiation of treatment.

In an alternative embodiment, the stabilization or improvement in the patient's health-related quality of life is assessed following a renal flare and then compared to the same patient's health-related quality of life prior to the renal flare. For instance, in some embodiments, the stabilization or improvement in an individual's health-related quality of life is evidenced by a maintained or increased domain score on the Medical Outcome Survey Short Form 36 (SF-36) in one or more of the domains selected from the group consisting of Physical Functioning, Role Physical, Bodily Pain, General Health Perception, Social Functioning, Vitality, Role Emotional, Mental Health. The increase in one or more individual domain scores selected from the group consisting of Physical Functioning, Role Physical, Bodily Pain, General Health Perception, Social Functioning, Vitality, Role Emotional, and Mental Health is optionally a clinically important increase of at least about 5 points, at least about 10 points, at least about 15 points, or at least about 20 points as compared to a baseline measurement. Alternatively, the increase in one or more individual domain scores selected from the group consisting of Physical Functioning, Role Physical, Bodily Pain, General Health Perception, Social Functioning, Vitality, Role Emotional, and Mental Health is optionally a clinically important increase of at least about 33% over baseline or at least about 36% over baseline.

In one embodiment of the invention, a quality of life enhancing medication is administered to the individual in addition to the epitope or epitope-presenting carrier. In some embodiments of the invention, the epitopes described herein are administered to the individual in an amount effective to reduce the amount of quality of life enhancing medication administered to the individual. The quality of life enhancing medication is optionally a psychiatric medication. For instance, the quality of life enhancing medication is optionally an antidepressant.

In another aspect, the invention provides a method of monitoring the health-related quality of life in an individual with SLE, comprising measuring the levels of circulating anti-dsDNA antibodies in the individual, wherein increased levels of circulating anti-dsDNA antibodies are indicative of decreased health-related quality of life and decreased levels of circulating anti-dsDNA antibodies are indicative of increased health-related quality of life. In other embodiments, the monitoring is effected by measuring and/or assessing any of the aspects or indicia of health-related quality of life as treatment progresses.

IV. Selection of Individuals for Treatment

Individuals for treatment are identified or indicated by any of a number of criteria. Individuals especially suitable for treatment are human. Individuals suitable for treatment have, have had, and/or are at risk of renal SLE disease. In some embodiments, individuals suitable for treatment have antibodies with high affinity to a dsDNA epitope (for example, LJP 394), and/or have significant renal impairment.

The present invention further provides methods of selecting individuals for treatment.

For instance, the methods of the present invention optionally comprise the additional step of assessing the health-related quality of life of an individual with SLE, wherein the health-related quality of life of an individual with SLE is used as a basis for selecting the individual to receive or continue to receive a method of treatment described. Methods of assessing the health related quality of life of an individual are described above and elsewhere herein.

Accordingly, the invention provides a method of stabilizing or improving the health-related quality of life in an individual with SLE comprising the steps of selecting an individual for receiving or continuing to receive treatment based on the individual's need for a stabilized or improved health-related quality of life, and administering a treatment to the selected individual, wherein administration of the treatment effects a sustained reduction of anti-dsDNA antibodies in the individual.

Optionally, the health-related quality of life of an individual may be assessed to determine whether or not an individual has a low health-related quality of life (LQOL) or a high health-related quality of life (HQOL). Individuals with low health-related quality of life experience a health-related quality of life that is below normal in one or more aspects. For instance, an individual with low health-related quality of life may be below normal in one or more aspects of health-related quality of life selected from the group consisting of limitations in physical activities because of health problems, limitations in social functioning because of physical or emotional problems, limitations in work or other usual activities because of physical health problems, bodily pain, general mental health, limitations in work or other usual activities because of emotional problems, vitality, and general health perception. Individuals having a low health-related quality of life may optionally be selected to receive or continue to receive treatment according to one of the methods of the present invention such as the reduction of circulating SLE-associated antibodies or administration of an epitope described herein.

In other embodiments of the invention, an individual is selected to receive or continue to receive treatment based on any of a wide variety of criteria. In some embodiments, significantly impaired renal function is used as a basis for administration of the treatment. In one embodiment an individual is selected to receive or continue to receive treatment based on the fact that the individual has an abnormally elevated serum creatinine level. Selecting those individuals having significantly impaired renal function may be on the basis of any clinical indication of significant renal impairment known in the art, including, but not limited to, anuria, oliguria, elevated serum creatinine levels, elevated BUN, proteinuria, hematuria (occult or gross), reduced creatinine clearance, impaired glomeral filtration, and the like. As will be apparent to one of skill in the art, a diagnosis of renal dysfunction, such as a diagnosis of subacute glomerulonephritis, nephrotic syndrome, or mild to severe nephritis, will also identify a significant impairment of renal function and thus serve as a basis for treating that individual and/or selection of the individual for treatment in accordance with the instant methods.

As will be apparent, the quantitative level of a particular clinical parameter that indicates a significant impairment of renal function will depend on the particular clinical parameter. Proteinuria is easily detected at a ‘screening’ level using colorimetric “dipstick” testing of urine, and can be followed up by more sensitive and accurate laboratory testing. Preferably, when the presence of a significant impairment of renal function is identified by proteinuria, an individual is considered to have significantly impaired renal function when at least about 500 mg of protein is excreted in the urine per day, more preferably at least about (i.e., greater than or equal to about) 1.5, 2, 2.5, 3, 3.5, 5.0, 6.0, 7.0, 8.0, 9.0, or 10 grams of protein per day. When serum creatinine is used as the indicator of significant impairment of renal function, an individual will be considered to have significantly impaired renal function when serum creatinine levels are at least about (i.e., greater than or equal to about) 1.5, 2, 2.5 3.0, 3.5, 4.0, 5.0, 6.0, 7.0, 8.0, 9.0, or 10 milligrams per deciliter (mg/dL). For instance, an individual will be considered to have significantly impaired renal function when serum creatinine levels are at least about 1.5 mg/dL. In addition, a clinically measurable hematuria level in an individual is optionally used as a basis for selecting an individual for the initiation or continuation of treatment.

Optionally, the indicators of significantly impaired renal function are apparent within about a three month period, about a six-month period, or about a one year period prior to treatment.

Alternatively, the individual may be selected to receive or continue to receive administration of the treatment based on evidence that the individual had a renal flare prior to the initial administration of the treatment, such as in the 3 month period, 6 month period, or 12 month period prior to the treatment.

In another embodiment, individuals with clinically defined renal disease based on a biopsy are selected for treatment.

In another embodiment, the methods further comprise the step of evaluating an individual for the presence of elevated levels of anti-dsDNA antibodies and then selecting the individual to receive or continue to receive the treatment based on the presence of elevated levels of anti-dsDNA in the individual.

In addition to methods of treatment which comprise the step of selecting an individual for treatment, the present invention also provides methods of identifying individuals suitable for treatment according to the methods described herein. For instance, in some embodiments, the invention provides a method of identifying an individual with SLE suitable for treatment using the methods described herein, comprising the steps of (i) assessing the health-related quality of life of an individual (ii) selecting the individual as suitable for treatment using the methods described herein if the individual has below normal health-related quality of life in one or more aspects, such as limitations in social functioning and/or limitations in work or other usual activities because of emotional problems. In some embodiments, the SF-36 is used for such an assessment. Other methods of assessment are described above.

Accordingly, the invention provides a method of identifying an individual (with SLE) suitable for receiving treatment according to the methods described herein, comprising the steps of (i) assessing one or more aspects of the health-related quality of life of the individual selected from the group consisting of limitations in physical activities because of health problems, limitations in social functioning because of physical or emotional problems, limitations in work or other usual activities because of physical health problems, bodily pain, general mental health, limitations in work or other usual activities because of emotional problems, vitality, and general health perception; and (ii) identifying an individual having a low health-related quality of life with respect to one or more of the above mentioned aspects of health-related quality of life (especially with respect to the aspects of limitations in work or other usual activities because of emotional problems and limitations in social functioning) as a suitable subject for receiving treatment.

In some embodiments, anti-dsDNA antibodies from individual bind with high affinity to the treatment modality (such as dsDNA epitope) and affinity is used as a basis for selecting the individual for treatment (receive and/or continue to receive treatment).

In one aspect, the invention provides a method of stabilizing or improving the health-related quality of life in an individual having SLE comprising the steps of selecting an individual to receive or continue to receive a dsDNA epitope based on the affinity of the dsDNA epitope (or a functional equivalent or correlate) for an anti-dsDNA antibody in the individual, and administering the dsDNA epitope to the selected individual, wherein administration of the dsDNA epitope stabilizes or improves the health-related quality of life in an individual.

As will be understood by one of skill in the art, administration of an effective amount of a dsDNA epitope, preferably in the form of an epitope-presenting carrier, and preferably wherein at least one of the epitopes is bound at a high initial affinity by antibodies from the patient entails assessing antibody affinity from an individual in those embodiments in which selection is based on antibody affinity, wherein said individual has, or is suspected of having, SLE. For purposes of this invention: (a) the affinity in question is with respect to an individual's antibodies, that is, antibodies obtained from that individual; (b) the antibody for which affinity is measured is an antibody associated with, and/or implicated in SLE; and (c) the binding of interest is binding of antibody to an epitope which binds to the antibody(ies), generally the epitope to be used in the proposed treatment, as described herein (i.e., a dsDNA epitope), or binding which correlates with binding of the epitope(s) to be used in the proposed treatment.

For all embodiments of the invention which use or are directed to K_{D}′, whether screening, treatment, monitoring, or any other methods directed to assessing affinity, it is understood that other, equivalent values can be measured and used, and are encompassed by this invention. For example, as discussed below, there are a number of methods known in the art which can measure (and express) affinity of antibodies from an individual for an epitope to be used for treatment (in the context of this invention, a double stranded DNA epitope). As is understood and conveyed by this disclosure, affinity may be measured using any epitope whose binding to the dsDNA antibody correlates with binding of the epitope(s) to be used in the proposed treatment (for example, a single-stranded counterpart of a double-stranded polynucleotide). K_{D}′ is one of these parameters, and equivalent parameters can be measured and used in this invention. Further, with respect to K_{D}′ cut-off values reported herein, the basis of this finding was administering about 100 mg of LJP 394 conjugate about once a week.

Measurement of affinity, either represented by measuring K_{D}′ or by some other method, either before or during treatment is strong, if not conclusive, indication that this parameter was a basis for selecting the individual to receive (and/or continue to receive) treatment. Accordingly, with respect to all treatment methods described herein, and as the definition for “is used as a basis” states, other embodiments include (1) assessing, or measuring, the affinity as described herein (and preferably selecting an individual suitable for receiving (including continuing to receive) treatment); and (2) administering the treatment(s) as described herein. As described herein, in some embodiments, more than one measurement is made, when change (if any) in affinity is assessed.

Antibody affinity may be measured using methods known in the art which assess degree of binding of a DNA epitope to an antibody. Generally, these methods comprise competition assays and non-competition assays. With respect to polynucleotide epitopes (which may be used in an epitope-presenting carrier), affinity may be measured using polynucleotide alone or polynucleotide-containing epitope-presenting carriers (as long as the polynucleotide and epitope-presenting carrier give equivalent, or at least convertible, values). Affinity may be measured using the epitope (or a molecule or moiety comprising the epitope) used in the epitope-presenting carrier; alternatively, a similar, non-identical epitope may be used, as long as its affinity may be at least correlated to the affinity of the epitope used in the conjugate, so that a meaningful measurement of affinity may be obtained.

In a competition assay, varying concentrations of antibody or epitope are reacted with epitope or antibody, and results may be expressed in terms of amount of antibody (generally in terms of concentration) required to reach half-maximal binding, generally designated as IC₅₀.

Another convenient way to express affinity is apparent equilibrium dissociation constant, or K_{D}′, which reflects the titer-weighted average affinity of the antibody for the antibody-binding epitope or epitope-presenting carrier. Antibody is generally obtained from whole blood and measured, by plasma, serum, or as an IgG fraction, and the affinity of this fraction for the epitope or epitope-presenting carrier is measured. Methods of obtaining IgG fractions are known in the art and are described herein. One preferred way to measure affinity is to measure KD′ based on a surface plasmon resonance assay.

Another way to measure affinity is by kinetic (i.e., non-equilibrium) analysis, methods of which are known in the art. Preferably, rate of dissociation (i.e., off rate) of antibody from epitope is measured.

In preferred embodiments, the affinity of the individual's antibodies for the dsDNA epitope(s) (whether measured directly using the epitope itself or using a moiety/epitope the affinity of which may be correlated to the affinity of the epitope used in the carrier) is measured as the apparent equilibrium dissociation constant (K_{D}′) for the dsDNA epitope(s) in the carrier before or upon initiation of treatment is less than about (in some embodiments, less than or equal to about) 1.0 mg IgG per mL. In other embodiments, the K_{D}′ is less than about (in some embodiments, less than or equal to about) any of the following: 0.8; 0.7; 0.6; 0.5; 0.4; 0.3; 0.2; 0.1; 0.09; 0.08; 0.07; 0.06; 0.05; 0.025. In some embodiments, K_{D}′ is less than about (in some embodiments, less than or equal to about) 0.8 mg IgG per mL. In some embodiments, K_{D}′ is less than or equal to about (in some embodiments, less than or equal to about) 0.5 mg IgG per mL. In some embodiments, K_{D}′ is less than about (in some embodiments, less than or equal to about) 0.1 mg IgG per mL. In some embodiments, the dsDNA epitope used comprises, consists essentially of, or consists of the double-stranded polynucleotide 5′-GTGTGTGTGTGTGTGTGTGT-3′(SEQ ID NO:1) in combination with its complementary strand, particularly the sequence 3′-CACACACACACACACACACA-5′(SEQ ID NO:2), or one of the single-stranded polynucleotides 5′-GTGTGTGTGTGTGTGTGTGT-3′(SEQ ID NO:1) or 3′-CACACACACACACACACACA-5′(SEQ ID NO:2), and the initial K_{D}′ is less than about 0.8 mg IgG per ml (in some embodiments, less than or equal to 0.8 mg IgG per ml). In some embodiments, the therapeutic moiety is LJP 394.

In some embodiments, an individual is considered to have high affinity for a dsDNA epitope if the antibody affinity of the individual is in a relatively high percentile ranking of affinity compared to a population. For example, there is a range of antibody affinities over a given patient population, and individuals considered to have high affinity for a dsDNA epitope can be identified based on a percentile ranking of antibody affinity with respect to this population. Accordingly, in some embodiments, an individual is considered to have high affinity antibodies if the antibody affinity relative to the dsDNA epitope(s) for that individual is greater than about the 20th percentile (i.e., in about the top 80% of affinities for that population), and considered to not have high affinity antibodies (i.e., is not selected for treatment in accordance with the invention) if the individual's antibody affinity is in or below the 20th percentile. In other embodiments, an individual is included in treatment, or identified as suitable to receive treatment, if the antibody for that individual is greater than about the 50th percentile for that population. In some embodiments, the individual is considered to have high affinity antibody if the affinity is greater than the 70th, 75th, 80th, 85th, 90th, or 95th percentile. A population may be about, or alternatively at least about any of the following, in terms of number of individuals measured: 10, 15, 20, 25, 30, 50, 60, 75, 100, 125, 150, 175, 200, 225, 250, 300, 400, 500. Preferably, a sufficient number of individuals are measured to provide a statistically significant population, which can be determined by methods known in the art. An upper limit of a population may be any number, including those listed.

Affinity may or may not change over the course of treatment. In some embodiments which include a step wherein the individual's antibody affinity for the dsDNA epitope(s) is remeasured after initiation of the treatment, the treatment may be continued if the average affinity of the individual's antibodies for the dsDNA epitope(s) is decreased by at least about 15%, preferably at least about 20%, more preferably at least about 25%, more preferably at least about 40%, more preferably at least about 50%, compared to the affinity measured before or at initiation of treatment, or may be discontinued if the antibody affinity has not decreased by at least about 15% (preferably at least about 20%, more preferably at least about 25%, more preferably at least about 40%, more preferably at least about 50%). For these embodiments, antibody affinity is measured after initiation of treatment (for comparison to antibody affinity before or upon initiation of treatment) at least about 4 weeks, preferably at least about 6 weeks, more preferably at least about 10 weeks, more preferably at least about 12 weeks, after initiation of treatment. In other embodiments, treatment may be continued if antibody affinity is decreased at least about any of the following (as compared to antibody affinity before or upon initiation of treatment): 40%, 50%, 75%, 100%, 200%, 500%. Preferably, antibody affinity is measured as the K_{D}′. As is understood by those of skill in the art, K_{D}′ values are inversely proportional to the affinity of the antibodies measured. Accordingly, in some embodiments, when K_{D}′ values are used to measure antibody affinity, treatment may be continued if the K_{D}′ increases by at least about 15%, and may be continued if K_{D}′ is increased at least about any of the following (as compared to antibody affinity before or upon initiation of treatment): 40%, 50%, 75%, 100%, 200%, 500%.

When antibody affinity is assayed using surface plasmon resonance, a reduction in affinity of at least about 15%, preferably at least about 20%, more preferably at least about 25%, more preferably at least about 40%, more preferably at least about 50% indicates responsiveness and that continuation of the treatment is indicated. For a competitive Farr assay, the same reductions in affinity generally apply. For other assays, the change can be at least about any of the above percentages, and further can be at least about any of the following percentages: 75%, 100%, 150%, 200%, 250%, 300%, 350%, 400%, 450%, 500%.

These methods may be practiced independently of the treatment methods, and may be practiced by a skilled technician other than a medical doctor, using equipment and/or techniques of the art.

V. Administration of Epitopes, Including Epitope-Presenting Carriers

Various formulations of ds-DNA epitopes (e.g., double-stranded DNA, or an analog or mimetic thereof), or epitope-presenting carriers comprising these epitopes, such as epitope-presenting valency platform molecules, may be used for administration, and, as such, the methods of this invention include administering a composition comprising any dsDNA epitope (e.g., double-stranded DNA, or an analog or mimetic thereof), epitope-presenting carrier, or epitope-presenting valency platform molecule(s) described herein. In some embodiments, the compositions may be administered “neat” (e.g., dissolved in pure water, such as USP water for injection). In some embodiments, the compositions comprise a conjugate(s) and a pharmaceutically acceptable excipient, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington's Pharmaceutical Sciences 19th Ed. Mack Publishing (1995) and Remington: The Science and Practice of Pharmacy, 20^{th} Ed., Lippincott, Williams & Wilkins (2000).

Generally, these compositions are formulated for administration by injection (e.g., intraperitoneally, intravenously, subcutaneously, intramuscularly, etc.). Accordingly, these compositions are preferably combined with pharmaceutically acceptable vehicles such as saline, Ringer's solution, dextrose solution, and the like, and, as is understood in the art, are usually sterile to be suitable for injection, especially in humans. Generally, the epitope or epitope-presenting carrier will normally constitute about 0.01% to 10% by weight of the formulation due to practical, empirical considerations such as solubility and osmolarity. The particular dosage regimen, i.e., dose, timing and repetition, will depend on the particular individual and that individual's medical history. Generally, a dose of about 1 μg to about 100 mg conjugate/kg body weight, preferably about 100 μg to about 10 mg/kg body weight, preferably about 150 μg to about 5 mg/kg body weight, preferably about 250 μg to about 1 mg conjugate/kg body weight. In other embodiments, these dosage ranges apply to other compositions used for treatment. Empirical considerations, such as the half life, generally will contribute to determination of the dosage. Other dosages, such as about 50 to 100 mg per week, 50 to 250 mg per week, and 50 to 500 mg per week (with any value inbetween the lower and upper limit of these ranges) are also contemplated. Example 1 provides an example of a dosing regimen. If used as a toleragen, conjugate may be administered daily, for example, in order to effect antibody clearance (pheresis), followed by less frequent administrations, such as two times per week, once a week, or even less frequently. Frequency of administration may be determined and adjusted over the course of therapy, and is based on maintaining tolerance (i.e., reduced or lack of immune response to dsDNA). Other appropriate dosing schedules may be as frequent as continuous infusion to daily or 3 doses per week, or one dose per week, or one dose every two to four weeks, or one dose on a monthly or less frequent schedule depending on the individual or the disease state. Repetitive administrations, normally timed according to B cell turnover rates, may be required to achieve and/or maintain a state of humoral anergy. Such repetitive administrations generally involve treatments of about 1 μg to about 10 mg/kg body weight or higher every 30 to 60 days, or sooner, if an increase in anti-dsDNA antibody level is detected. Alternatively, sustained continuous release formulations of the compositions may be appropriate. Various formulations and devices for achieving sustained release are known in the art.

In some embodiments, LJP 394, a dsDNA epitope presenting valency platform molecule described below, is formulated as a sterile, colorless liquid in an isotonic phosphate-buffered saline solution for intravenous (IV) administration. Each 1 mL of solution contains 50 mg of LJP 394, 1.9 mg Na₂HPO₄*7H₂O, 0.30 mg NH₂PO₄*H₂O, and 5.8 mg NaCl in water for Injection, USP (pH 6.8-8.0). The formulation contains no preservatives. Other formulations are designed to be 20 mg/mL, 10 mg/mL, and 1 mg/mL of LJP 394. The formulations are preferably stored at cooler temperatures, such as 2 to 8° C. In other embodiments, each 1 mL of solution contains 50 mg of LJP 394, 1.9 mg Na₂HPO₄*7H₂O, 0.30 mg NH₂PO₄*H₂O, and 8.0 mg NaCl in water for Injection, USP (pH 6.8-8.0). LJP 394 is also optionally administered as 100 mg in 2 ml. As described herein, LJP 394 may be administered as 100 mg weekly.

Due to the chronic nature of systemic lupus erythematosus, the epitopes, epitope-presenting carriers, and epitope-presenting valency platform molecules, including conjugates, of the present invention will in some embodiments be administered to patients for extended periods of time. For instance, in some embodiments, the dsDNA epitope or the conjugate comprising a non-immunogenic valency platform molecule and two or more double-stranded DNA epitopes is administered to an individual for more than 72 weeks. In another embodiment, the dsDNA epitope or the conjugate is administered on a weekly basis for more than 16 weeks. In still another embodiment, LJP 394 is administered to an individual at a dosage of 50 mg to 200 mg weekly for a period of more than 16 consecutive weeks. Optionally, LJP 394 is administered at a dosage of 100 mg weekly for more than 16 weeks.

Other formulations include those suitable for oral administration, which may be suitable if the conjugate is able to cross the mucosa. Similarly, an aerosol formulation may be suitable.

Other formulations include suitable delivery forms known in the art including, but not limited to, carriers such as liposomes. Mahato et al. (1997) Pharm. Res. 14:853-859. Liposomal preparations include, but are not limited to, cytofectins, multilamellar vesicles and unilamellar vesicles.

In some embodiments, more than one epitope or epitope-presenting carrier may be present in a composition. Such compositions may contain at least one, at least two, at least three, at least four, at least five different epitopes or epitope-presenting carriers. Such “cocktails”, as they are often denoted in the art, may be particularly useful in treating a broader range of population of individuals. They may also be useful in being more effective than using only one (or fewer than are contained in the cocktail) conjugate(s).

The compositions may be administered alone or in conjunction with other forms of agents that serve to enhance and/or complement the effectiveness of a epitope or epitope-presenting carrier of the invention, including, but not limited to, anti-T cell treatments. Such treatments usually employ agents that suppress T cells such as steroids or cyclosporin. Other agents are corticosteroid and/or cyclophosphamide immunosuppressive therapy. Other possible agents which may be administered in combination with the epitopes, epitope-presenting carriers, or epitope-presenting valency platform molecules are psychiatric medications, such as antidepressants.

VI. Treatment Modalities

Any agent which can effect reduction of anti-dsDNA antibodies is suitable for this invention. More desirably, an agent which selectively reduces the level of circulating anti-dsDNA antibodies in an individual is used. In some embodiments, the agent is not broadly immunosuppressant.

A. Epitopes

Epitopes used in the methods of the present invention comprise dsDNA epitopes, such as double-stranded DNA, or analogs or mimetics thereof.

In some embodiments, dsDNA epitopes are used in the methods of the invention.

Double-stranded DNA (dsDNA) epitopes for use in the methods of the present invention may be any chemical moiety which specifically binds to a dsDNA antibody. In particular, epitopes of interest include those that bind the anti-polynucleotide (particularly anti-DNA, including anti-double stranded DNA) antibodies that occur in systemic lupus erythematosis. Generally, but not necessarily, the dsDNA epitopes used are polynucleotides, optionally DNA (including DNA analogs), and optionally double-stranded DNA or optionally single-stranded DNA.

Examples of suitable epitopes are described, for instance, in U.S. Pat. Nos. 5,162,515; 5,391,785; 5,276,013; 5,786,512; 5,726,329; 5,552,391; 5,268,454; 5,633,395; 5,606,047.

For instance, optionally, the polynucleotide is double-stranded DNA. In some embodiments, the polynucleotide comprises, consists essentially of, or consists of the double-stranded sequence 5′-GTGTGTGTGTGTGTGTGTGT-3′(SEQ ID NO:1) in combination with the complementary polynucleotide sequence, particularly the sequence 3′-CACACACACACACACACACA-5′(SEQ ID NO:2).

In some embodiments, the polynucleotide is single-stranded DNA comprising, consisting essentially of, or consisting of the sequence 5′-GTGTGTGTGTGTGTGTGTGT-3′ (SEQ ID NO:1). In some alternative embodiments, the polynucleotide is single-stranded DNA comprising, consisting essentially of, or consisting of the sequence 3′-CACACACACACACACACACA-5′ (SEQ ID NO:2).

In an alternative embodiment, the dsDNA epitope not only preferentially binds antibodies that preferentially bind double-stranded DNA, but also preferentially binds antibodies that preferentially bind a mimetic of dsDNA epitope, such as a polypeptide mimetic. In some embodiments, the dsDNA epitope preferentially binds an antibody that preferentially binds an NR2 receptor, such as N-methyl-D-aspartate (NMDA) receptor NR2a and/or N-methyl-D-aspartate (NMDA) receptor NR2b. In some embodiments, the mimetic of the dsDNA epitope comprises, consists essentially of, or consists of the pentapeptide sequence Asp/Glu-Trp-Asp/Glu-Tyr-Ser/Gly.

The suitability of particular epitopes for binding antibodies according to this invention can be identified and/or confirmed using techniques known in the art and described herein. For example, to select the optimum epitope from a library of small drug molecules believed to mimic the dsDNA epitope for SLE, a family of platforms can be constructed in which each of the candidates is alternatively displayed on a similar platform molecule. The composition is then tested for efficacy. For example, for in vivo use, an animal model is used in which there are circulating anti-DNA antibodies, such as, for example, the BXSB mouse model system. The animals can be immunized with an appropriate epitope to initiate the antibody response, if necessary. Test candidates assembled onto a platform are then used to treat separate animals, either by administration, or by ex vivo use, according to the intended purpose. The animals are bled before and after treatment, and the antibody levels in plasma are determined by standard immunoassay as appropriate for the specific antibody. Efficacy of the candidates is then assessed according to antibody affinity assays designed to indicate antibodies specific for the epitope being tested. Appropriate affinity assays are described herein.

Polynucleotides (or other dsDNA epitope candidates such as polypeptides) may be screened for binding activity with antisera containing the antibodies of interest, for example, SLE antisera, by the assays known in the art. Examples of such assays include competitive affinity assays, for example, a competitive Farr assay and/or a competitive ELISA assay, and/or non-competitive, equilibrium affinity assay, such as the surface plasmon resonance (for example, using BIACORE®) based assay as known in the art and as described herein and in WO 01/41813.

Antibody affinity may be measured using methods known in the art which assess degree of binding of DNA epitope to antibody. Generally, these methods comprise competition assays and non-competition assays. With respect to polynucleotide epitopes (which will be used in a conjugate to be administered), affinity may be measured using polynucleotide alone or polynucleotide-containing conjugates (as long as the polynucleotide and conjugate give equivalent, or at least convertible, values).

A competitive Farr assay is an exemplary assay. In a competition assay, varying concentrations of antibody or epitope are reacted with epitope or antibody, and results may be expressed in terms of amount of antibody (generally in terms of concentration) required to reach half-maximal binding, generally designated as IC₅₀. Polynucleotide duplexes having an IC₅₀ of less than about 500 nM, preferably less than 50 nM, are deemed to have significant binding activity and are, therefore, useful for making the conjugates of this invention (or, in other embodiments, useful in any other form as a dsDNA epitope).

Another convenient way to express affinity is apparent equilibrium dissociation constant, or K_{D}′, which reflects the titer-weighted average affinity of the antibody for the antibody-binding epitope on the conjugate. Antibody is generally obtained from whole blood and measured, by plasma, serum, or as an IgG fraction, and the affinity of this fraction for the conjugate is measured. Methods of obtaining IgG fractions are known in the art and are described herein. One preferred way to measure affinity is to measure K_{D}′ based on a surface plasmon resonance assay.

Another way to measure affinity is by kinetic (i.e., non-equilibrium) analysis, methods of which are known in the art. Preferably, rate of dissociation (i.e., off rate) of antibody from epitope is measured.

In one embodiment the apparent equilibrium dissociation constant (K_{D}′) for each of the double-stranded DNA epitopes with respect to the antibody to which it specifically binds is less than about 1.0 mg IgG per ml. In some other embodiments of the invention the apparent equilibrium dissociation constant (K_{D}′) for each of the double-stranded DNA epitopes with respect to the antibody to which it specifically binds is less than about 0.8 mg IgG per ml, less than about 0.5 mg IgG per ml, or less than about 0.2 mg IgG per ml. In other embodiments, the K_{D}′ is less than or equal to about any of these values.

It is understood that, for purposes of this invention, more than one type of dsDNA epitope(s) may be used in preparing a conjugate. Alternatively, one type (i.e., one chemical species) of a dsDNA epitope may be used. If a polynucleotide (such as dsDNA) is used, generally the length is greater than about 10 base pairs (bp), more preferably greater than about 15 bp, more preferably greater than or equal to about 20 bp. Generally, but not necessarily, the length is less than about 1 kb, preferably less than about 500 bp, preferably less than about 100 bp. It is understood that these values also pertain to single-stranded forms.

In some alternative embodiments, a mimetic of double-stranded DNA is used in the methods and compositions of the invention. For instance, in some embodiments, an NR2 receptor epitope, an exemplary mimetic of double-stranded DNA, is used in the methods of the invention. In some embodiments, the epitope the NR2 receptor epitope preferentially binds an SLE-associated antibody that preferentially binds the N-methyl-D-aspartate (NMDA) receptor NR2a and/or N-methyl-D-aspartate (NMDA) receptor NR2b. In some embodiments, the NR2 receptor epitope comprises, consists essentially of, or consists of the pentapeptide sequence Asp/Glu-Trp-Asp/Glu-Tyr-Ser/Gly.

B. Epitope-Presenting Carriers

In some embodiments, the dsDNA epitope (or the mimetic or analog thereof) administered to an individual with SLE in any of the methods described herein is administered in the form of an epitope-presenting carrier.

Any of a variety of carriers may be used, as long as the carrier does not elicit an undesirable or unacceptable immune response. The carrier may be any chemical moiety, and have any chemical structure, including, but not limited to, organic and inorganic molecules, polypeptides (i.e., polymers of amino acids), nucleic acids, carbohydrates, other polymers, artificial structures, and lipid structures (such as liposomes or micelles) made by standard techniques, or polymerized as described in U.S. Pat. No. 5,512,294.

In some embodiments, the epitope-presenting carrier comprises more than one attached or bound epitopes. Optionally, the epitope-presenting carrier is an epitope-presenting valency platform molecule. Exemplary epitope-presenting valency platform molecules are described below.

C. Epitope-Presenting Valency Platform Molecules

In some embodiments, epitope-presenting valency platform molecule are used in the methods of the invention. In some embodiments, the epitope-presenting valency platform molecule is a conjugate comprising a non-immunogenic valency platform molecule and at least two (i.e., two or more) dsDNA epitopes, optionally polynucleotides which bind to anti-dsDNA antibody from the individual.

Any of a variety of non-immunogenic valency platform molecules (also called “platforms”) may be used in the conjugates of the invention. Many have been described in the art, such as polymers, and need not be described herein. Any non-immunogenic, acceptably low to non-toxic molecule which provides requisite attachment sites such that the conjugate may act to bind circulating anti-ds DNA antibody and/or induce B cell anergy and/or apoptosis in cells producing these antibodies may be used. Preferably, the conjugates comprise a chemically defined valency platform molecule in which a precise valency (as opposed to an average) is provided. Accordingly, a defined valency platform is a platform with defined structure, thus a defined number of attachment points and a defined valency. Certain classes of chemically defined valency platforms, methods for their preparation, conjugates comprising them and methods for the preparation of such conjugates suitable for use within the present invention include, but are not limited to, those described in the U.S. Pat. Nos. 5,162,515; 5,391,785; 5,276,013; 5,786,512; 5,726,329; 5,268,454; 5,552,391; 5,606,047; 5,663,395; and 6,060,056; and in commonly-owned U.S. Ser. Nos. 60/111,641 (U.S. Ser. No. 09/457,607, U.S. Pat. No. 6,458,953, and PCT App. No. PCT/US99/29339); 60/138,260 (U.S. Ser. No. 09/590,592 and PCT App. No. PCT/US00/15968), U.S. Ser. No. 09/457,913 (U.S. Pat. No. 6,399,578) (PCT App. No. PCT/US99/29338), U.S. Ser. No. 09/457,607 (U.S. Pat. No. 6,458,953) (PCT/US99/29339) and U.S. Ser. No. 09/877,387 (PCT/US01/18446), all of which are hereby incorporated by reference.

A platform may be proteinaceous or non-proteinaceous (i.e., organic). Examples of proteinaceous platforms include, but are not limited to, albumin, gammaglobulin, immunoglobulin (IgG) and ovalbumin. Borel et al. (1990) Immunol. Methods 126:159-168; Dumas et al. (1995) Arch. Dematol. Res. 287:123-128; Borel et al. (1995) Int. Arch. Allergy Immunol. 107:264-267; Borel et al. (1996) Ann. N.Y. Acad. Sci. 778:80-87.

The valency of a chemically-defined valency platform molecule within the present invention can be predetermined by the number of branching groups added to the platform molecule. Suitable branching groups are typically derived from diamino acids, triamines, and amino diacids.

Preferred valency platform molecules are biologically stabilized, i.e., they exhibit an in vivo excretion half-life often of hours to days to months to confer therapeutic efficacy, and are preferably composed of a synthetic single chain of defined composition. They generally have a molecular weight in the range of about 200 to about 200,000, preferably about 200 to about 50,000 (or less, such as 30,000). Examples of valency platform molecules within the present invention are polymers (or are comprised of polymers) such as polyethylene glycol (PEG), poly-D-lysine, polyvinyl alcohol, polyvinylpyrrollidone, D-glutamic acid and D-lysine (in a ratio of 3:2). Preferred polymers are based on polyethylene glycols (PEGs) having a molecular weight of about 200 to about 8,000, or, in some embodiments, about 200 to about 10,000. In other embodiments, the molecular weight can range between about 40,000 to about 100,000; with a range of about 10,000 to about 20,000 as preferable. Other suitable platform molecules for use in the conjugates of the invention are albumin and IgG. Valency platform molecules should be of a size such that a conjugate made with the valency platform does not become a T cell independent immunogen.

Preferred valency platform molecules suitable for use within the present invention are the chemically-defined valency platform molecules disclosed, for example, in co-owned U.S. Pat. No. 5,552,391, hereby incorporated by reference. These platforms generally have low polydispersity. Particularly preferred homogeneous chemically-defined valency platform molecules suitable for use within the present invention are derivatized 2,2′-ethylenedioxydiethylamine (EDDA) and triethylene glycol (TEG). The AHAB-TEG platform used for LJP 394 (a monodisperse platform) is described below.

In some embodiments, the valency platform molecules have the advantage of having a substantially homogeneous (i.e., uniform) molecular weight (as opposed to polydisperse molecular weight). Accordingly, a population of these molecules (or conjugates thereof) are substantially monodisperse, i.e., have a narrow molecular weight distribution. A measure of the breadth of distribution of molecular weight of a sample of a platform molecule (such as a composition and/or population of platform molecules) is the polydispersity of the sample. Polydispersity is used as a measure of the molecular weight homogeneity or nonhomogeneity of a polymer sample. Polydispersity is calculated by dividing the weight average molecular weight (Mw) by the number average molecular weight (Mn). The value of Mw/Mn is unity for a perfectly monodisperse polymer. Polydispersity (Mw/Mn) is measured by methods available in the art, such as gel permeation chromatography. The polydispersity (Mw/Mn) of a sample of valency molecules is preferably less than about 2, more preferably, less than about 1.5, or less than about 1.2, less than about 1.1, less than about 1.07, less than about 1.02, or, e.g., about 1.05 to 1.5 or about 1.05 to 1.2. Typical polymers generally have a polydispersity of about 2-5, or in some cases, 20 or more. Advantages of the low polydispersity property of these valency platform molecules include improved biocompatibility and bioavailability since the molecules are substantially homogeneous in size, and variations in biological activity due to wide variations in molecular weight are minimized. The low polydispersity molecules thus are pharmaceutically optimally formulated and easy to analyze. Accordingly, in some embodiments, the valency platform molecules have very low polydispersity, and, in some embodiments are monodisperse.

Preferred platforms for dsDNA epitopes are tetrabromoacetyl compounds, and other tetravalent and octavalent valency platform molecules, such as those described in Jones et al. (1995) J. Med. Chem. 38:2138-2144; and U.S. Patent references provided above.

Additional suitable valency platform molecules include, but are not limited to, tetraaminobenzene, heptaaminobetacyclodextrin, tetraaminopentaerythritol, 1,4,8,11-tetraazacyclotetradecane (Cyclam) and 1,4,7,10-tetraazacyclododecane (Cyclen).

In some embodiments, a platform having a defined number of attachment sites also comprises a (one or more) polyethylene oxide group, as described, for example, in U.S. patents and patent applications described above as well as U.S. Ser. No. 09/877,387, filed Jun. 7, 2001 (PCT/US01/18446). The molecular weight of PEG can be any molecular weight, including, but not limited to, greater than about 200, 500, 1000, 2000, 5000, 10,000, 15,000, 18,000, 22,000, 40,000, 50,000, 80,000, 100,000 Daltons. In some embodiments, in the valency platform molecule, the high molecular weight polyethylene oxide group has the formula:
—(CH₂CH₂O)ₙ—
wherein n is greater than 500; n is greater than 400; n is greater than 500; n is greater than 600; n is greater than 700; or n is greater than 800. In another embodiment, the valency platform molecule comprises a core group and at least three arms wherein each arm comprises a terminus. The core group and/or the arms may comprise a high molecular weight polyethylene oxide group. The high molecular weight polyethylene oxide group also may be attached to the core or arm. In some embodiments, a composition comprising the valency platform molecules is provided, wherein the molecules have a polydispersity less than 1.2. In another embodiment, the valency platform molecule may comprise at least three reactive conjugating groups such as hydroxyl, thiol, isocyanate, isothiocyanate, amine, alkyl halide, alkylmercurial halide, aldehyde, ketone, carboxylic acid halide, α-halocarbonyl, α,β-unsaturated carbonyl, haloformate ester, carboxylic acid, carboxylic ester, carboxylic anhydride, O-acyl isourea, hydrazide, maleimide, imidate ester, sulfonate ester, sulfonyl halide, α,β-unsaturated sulfone, aminooxy, semicarbazide, or β-aminothiol. In another embodiment, the valency platform molecule comprises at least 3 aminooxy groups and/or at least 3 carbamate groups.

In general, these platforms are made by standard chemical synthesis techniques. PEG must be derivatized and made multivalent, which is accomplished using standard techniques. Some substances suitable for conjugate synthesis, such as PEG, albumin, and IgG are available commercially.

For purposes of this invention, the valency platform molecules have a minimum valency of at least two, preferably at least four, preferably at least six, more preferably at least eight, preferably at least 10, preferably at least 12. As an upper limit, valency is generally less than 128, preferably less than 64, preferably less than 35, preferably less than 30, preferably less than 25, preferably less than 24, preferably less than 20, although the upper limit may exceed 128. Conjugates may also have valency of ranges of any of the lower limits of 2, 4, 6, 8, 10, 12, 16, with any of the upper limits of 128, 64, 35, 30, 25, 24, 20.

In some embodiments, the valency platform molecule comprises a carbamate linkage, i.e., —O—C(═O)—N<). Such platforms are described in a co-owned patent application entitled “Valency Platform Molecules Comprising Carbamate Linkages” U.S. Ser. No. 60/111,641 (U.S. Ser. No. 09/457,607 (U.S. Pat. No. 6,458,953) and PCT App. No. PCT/US99/29339), hereby incorporated by reference. Other valency platform molecules are described in the co-owned patent application entitled “Multivalent Platform Molecules Comprising High Molecular Wight Polyethylene Oxide,” U.S. Ser. No. 09/877,387 (U.S. Publication No. 2002/0110535).

In other embodiments, valency platforms may be used which, when conjugated, provide an average valency (i.e., these platforms are not precisely chemically defined in terms of their valency). Examples of such platforms are polymers such as linear PEG; branched PEG; star PEG; polyamino acids; polylysine; proteins; amino-functionalized soluble polymers.

In some embodiments, the conjugates include branched, linear, block, and star polymers and copolymers, for example those comprising polyoxyalkylene moieties, such as polyoxyethylene molecules, and in particular polyethylene glycols. The polyethylene glycols preferably have a molecular weight less than about 10,000 daltons. In some embodiments, polymers with low polydispersity may be used. For example, polyoxypropylene and polyoxyethylene polymers and copolymers, including polyethylene glycols may be modified to include aminooxy groups, wherein the polymers have a low polydispersity, for example, less than 1.5, or less than 1.2 or optionally less than 1.1 or 1.07. Preferably, the polymers comprise at least 3 aminooxy groups, or at least 4, 5, 6, 7, 8, or more.

D. Conjugation of Epitope(s) with Carriers

Conjugation of a biological or synthetic molecule to a carrier, such as a valency platform molecule, may be effected in any number of ways, including covalent and non-covalent, typically involving one or more crosslinking agents and functional groups on the biological or synthetic molecule and valency platform molecule. Examples of standard chemistry which may be used for conjugation include, but are not limited to: 1) thiol substitution; 2) thiol Michael addition; 3) amino alkylation (reductive alkylation of amino groups); 4) disulfide bond formation; 5) acylation of amines.

The synthetic polynucleotide duplexes that are coupled to a carrier, such as a valency platform molecule, are composed of at least about 20 bp and preferably 20-50 bp. As described herein, single-stranded forms may also be used. Reference to double-stranded forms is provided as an exemplary embodiment, with the understanding that appropriate principles are applicable to single-stranded forms. Further, linking other types of dsDNA epitope moieties uses techniques known in the art. Polynucleotides described herein are deoxyribonucleotides unless otherwise indicated and are set forth in 5′ to 3′ orientation. Preferably the duplexes are substantially homogeneous in length; that is, the variation in length in the population will not normally exceed about ±20%, preferably ±10%, of the average duplex length in base pairs. They are also preferably substantially homogeneous in nucleotide composition; that is, their base composition and sequence will not vary from duplex to duplex more than about 10%. Most preferably they are entirely homogeneous in nucleotide composition from duplex to duplex.

Based on circular dichroic (CD) spectra interpretation, in some embodiments, duplexes that are useful in the invention assume a B-DNA type helical structure. It should be understood that it is not intended that the invention be limited by this belief and that the duplexes may, upon more conclusive analysis assume Z-DNA and/or A-DNA type helical structures.

These polynucleotide duplexes may be synthesized from native DNA or synthesized by chemical or recombinant techniques. Naturally occurring or recombinantly produced dsDNA of longer length may be digested (e.g., enzymatically, chemically and/or by mechanical shearing) and fractionated (e.g., by agarose gel or Sephadex™ column) to obtain polynucleotides of the desired length.

Alternatively, pairs of complementary single-stranded polynucleotide chains up to about 70 bases in length are readily prepared using commercially available DNA synthesizers and then annealed to form duplexes by conventional procedures. Synthetic dsDNA of longer length may be obtained by enzymatic extension (5′-phosphorylation followed by ligation) of the chemically produced shorter chains.

The polynucleotides may also be made by molecular cloning. For instance, polynucleotides of desired length and sequence are synthesized as above. These polynucleotides may be designed to have appropriate termini for ligation into specific restriction sites. Multiple iterations of these oligomers may be ligated in tandem to provide for multicopy replication. The resulting construct is inserted into a standard cloning vector and the vector is introduced into a suitable microorganism/cell by transformation. Transformants are identified by standard markers and are grown under conditions that favor DNA replication. The polynucleotides may be isolated from the other DNA of the cell/microorganism by treatment with restriction enzymes and conventional size fractionation (e.g., agarose gel, Sephadex™ column).

Alternatively, the polynucleotides may be replicated by the polymerase chain reaction (PCR) technology. Saiki et al. (1985) Science 230:1350-1354; Saiki et al. (1988) Science 239:487-491; Sambrook et al. (1989) p 14.1-14.35.

In some embodiments, the polynucleotides are conjugated to a chemically-defined valency platform molecule in a manner that preserves their antibody binding activity. This is done, for example, by conjugating the polynucleotide to the valency platform molecule at a predetermined site on the polynucleotide chain such that the polynucleotide forms a pendant chain of at least about 20 base pairs measured from the conjugating site to the free (unattached) end of the chain.

In some embodiments, the polynucleotide duplexes are substantially homogenous in length and one strand of the duplex is conjugated to the carrier or valency platform molecule either directly or via a linker molecule. Synthetic polynucleotides may be coupled to a linker molecule before being conjugated to a carrier or valency platform molecule. Usually the linker containing strand of the duplex is coupled at or proximate (i.e., within about 5 base pairs) to one of its ends such that each strand forms a pendant chain of at least about 20 base pairs measured from the site of attachment of the strand to the linker molecule. The second strand is then annealed to the first strand to form a duplex. Thus, a conjugate within the present invention may be generally described by the following formula: [(PN)ₙ-linker]ₘ-valency platform molecule wherein PN=a double-stranded polynucleotide with “n” nucleotides, wherein n=at least about 20 and m=2-8. In other embodiments, n may have lower values.

In some embodiments, the polynucleotides of the conjugates are coupled to a linker molecule at or proximate one of their ends. The linker molecule is then coupled to the carrier or valency platform molecule. As described in U.S. Pat. No. 5,552,391 and incorporated herein by reference, exemplary of suitable linker molecules within the present invention are 6 carbon thiols such as HAD, a thio-6 carbon chain phosphate, and HAD_{P}S, a thio-6 carbon chain phosphorothioate. Chemically-defined valency platform molecules within the present invention are formed, for example, by reacting amino modified-PEG with 3,5-bis-(iodoacetamido) benzoyl chloride (hereinafter “IA-DABA”); 3-carboxypropionamide-N,N-bis-[(6′-N′-carbobenzyloxyaminohexyl)acetamide] 4″-nitrophenyl ester (hereinafter “BAHA”); 3-carboxypropionamide-N,N-bis-[(8′-N′-carbobenzyloxyamino-3′,6′-dioxaoctyl)acetamide] 4″-nitrophenyl ester (hereinafter “BAHA_{OX}”); or by reacting PEG-bis-chloroformate with N,N-di(2-[6′-N′-carbobenzyloxyaminohexanoamido]ethyl)amine (hereinafter “AHAB”) to form chemically-defined valency platform molecules.

For example, a defined double-stranded polynucleotide (PN) can be conjugated to a valency platform molecule by first providing a single chain consisting of approximately 20 alternating cytosine (C) and adenosine (A) nucleotides. Four C A chains may then be covalently conjugated through linkers such as HAD to four reactive sites on a derivatized platform molecule such as triethylene glycol. The valency platform molecule is synthesized to include groups such as bromoacetyl. During the conjugation, a leaving group is displaced by sulfur. A second single nucleotide chain consisting of approximately 20 alternating thymidine (T) and guanosine (G) nucleotides can then be annealed to the CA strand to form a double-stranded PN conjugate of the formula, [(PN)₂₀-linker]₄-valency platform molecule.

Alternatively, in another embodiment, the polynucleotide may be coupled to the derivatized valency platform molecule at the 3′ end of the polynucleotide via a morpholino bridge formed by condensing an oxidized 3′ terminal ribose on one of the strands of the polynucleotide with a free amino group on the derivatized platform molecule and then subjecting the adduct to reducing conditions to form the morpholino linkage, as described in U.S. Pat. No. 5,553,391. Such coupling requires the derivatized platform molecule to have at least an equal number of amino groups as the number of polynucleotide duplexes to be bound to the platform molecule. The synthesis of such a conjugate is carried out in two steps. The first step is coupling one strand of the polynucleotide duplex to the derivatized platform molecule via a condensation/reduction reaction. The oxidized 3′ terminal ribose is formed on the single polynucleotide strand by treating the strand with periodate to convert the 3′ terminal ribose group to an oxidized ribose group. The single-stranded polynucleotide is then added slowly to an aqueous solution of the derivatized platform molecule with a pH of about 6.0 to 8.0 at 2-8° C., generally with a reducing agent (such as sodium borohydride).

The molar ratio of polynucleotide to platform molecule in all the conjugation strategies will normally be in the range of about 2:1 to about 30:1, usually about 2:1 to about 8:1 and, in some embodiments, about 4:1 to 6:1. In this regard, it is preferable that the conjugate not have an excessively large molecular weight as large molecules, particularly those with repeating units, of m.w. >200,000 may be T-independent immunogens. See Dintzis et al. (1983) J. Immunol. 131:2196 and Dintzis et al. (1989) J. Immunol. 143:1239. During or after the condensation reaction (normally a reaction time of 24 to 48 hr), a strong reducing agent, such as sodium cyanoborohydride, is added to form the morpholino group. The complementary strand of the duplex is then added to the conjugate and the mixture is heated and slowly cooled to cause the strands to anneal. The conjugate may be purified by gel permeation chromatography.

An alternative to the ribose strategy is forming aldehyde functionalities on the polynucleotides and using those functionalities to couple the polynucleotide to the carrier or platform molecule via reactive functional groups thereon. Advantage may be taken of the fact that gem vicinal diols, attached to the 3′ or 5′ end of the polynucleotide, may be oxidized with sodium periodate to yield aldehydes which can condense with functional amino groups of the platform molecule. When the diols are in a ring system, e.g., a five-membered ring, the resulting condensation product is a heterocyclic ring containing nitrogen, e.g., a six-membered morpholino or piperidino ring. The imino-condensation product is stabilized by reduction with a suitable reducing agent; e.g., sodium borohydride or sodium cyanoborohydride. When the diol is acyclic, the resulting oxidation product contains just one aldehyde and the condensation product is a secondary amine.

Another procedure involves introducing alkylamino or alkylsulfhydryl moieties into either the 3′ or 5′ ends of the polynucleotide by appropriate nucleotide chemistry, e.g., phosphoramidite chemistry. The nucleophilic groups may then be used to react with a large excess of homobifunctional cross-linking reagent, e.g., dimethyl suberimidate, in the case of alkylamine derivatives, or an excess of heterobifunctional cross-linking reagent, e.g., m-maleimidobenzoyl-N-hydroxysuccinimide ester (MBS) or succinimidyl (4-iodoacetyl) aminobenzoate (SIAB), for the alkylsulfhydryl derivatives. Once excess cross-linker is removed, the polynucleotide derivatives are reacted with amino groups on the platform molecule. Alternatively, the sulfhydryl group may be reacted with an electrophilic center on the platform, such as a maleimide or α-haloacetyl group or other appropriate Michael acceptor.

Still another strategy employs modified nucleosides. Suitable deoxynucleoside derivatives can be incorporated, by standard DNA synthetic chemistry, at desired positions in the polynucleotide, preferably on the 5′ or 3′ ends. These nucleoside derivatives may then react specifically and directly with alkylamino groups on the carrier or platform molecule. Alternatively, side reactions seen with the above-described dialdehyde chemistry, such as amine catalyzed beta-elimination, can be circumvented by employing appropriate nucleoside derivatives as the 3′ terminus of the chain to be attached. An example of this is 5′ methylene extension of ribose; i.e., a 5′(2-hydroxyethyl)-group instead of a 5′ hydroxymethyl group. An alternative would be to use a phosphonate or phosphinate linkage for the 3′ terminal dinucleotide of the polynucleotide to be attached to the carrier or platform molecule.

A description of the synthesis of the conjugate LJP 394, a tetravalent conjugate, is described in Jones et al. (1995) and in U.S. Pat. No. 5,552,391, which are hereby incorporated by reference. LJP 394 comprises four 20-mer oligonucleotides consisting of alternating C and A nucleotides, (CA)₁₀, attached to a platform and annealed with complementary 20-mer oligonucleotides consisting of alternating G and T nucleotides, (GT)₁₀, oligonucleotide. The valency platform molecule used in LJP 394 is shown immediately below. In some embodiments, the platform molecule is 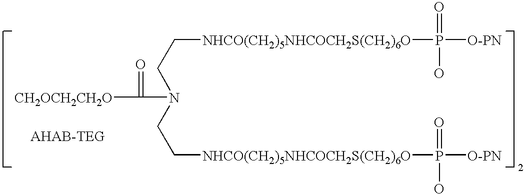
wherein PN is the polynucleotide. Accordingly, the epitope-presenting valency platform molecule administered to individuals with SLE in some embodiments of any of the methods of the invention described herein is LJP394 (also referred to herein as “Riquent”™), which comprises a molecule of the following formula: 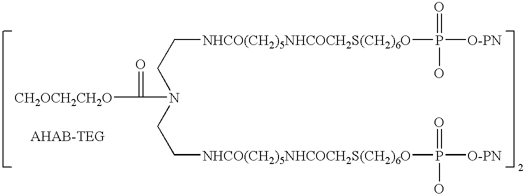
wherein PN is (CA)₁₀·(TG)₁₀. In some embodiments, pharmaceutically acceptable salts (e.g., sodium salts) of the molecules described herein are administered to individual with SLE. A variety of pharmaceutically acceptable salts are known to those of ordinary skill in the art.
VII. Kits for Effecting Treatment

The invention also provides kits for effecting treatment using the methods of the present invention. The kits comprise an epitope described herein, optionally in the form of an epitope-presenting carrier, optionally in the form of an epitope-presenting valency platform molecule. In some embodiments, the kit comprises a pharmaceutical composition comprising (i) an epitope, optionally in the form of an epitope-presenting carrier such as an epitope-presenting valency platform molecule, and (ii) a pharmaceutically acceptable excipient. In some embodiments, the kits further comprise suitable packaging and/or instructions for use of the epitope, or pharmaceutical composition thereof, in accordance with the methods of treatment described herein. The instructions included in the kit may include, but are not necessarily limited to, instructions describing the administration of the epitope, or pharmaceutical composition thereof, to an individual to stabilize or improve the health-related quality of life of the individual. Optionally, the instructions comprise a description of selecting an individual suitable for treatment with the epitope, or pharmaceutical composition thereof, based on the low health-related quality of life of the individual.

Thus, in some embodiments, the invention provides a kit comprising (a) a dsDNA epitope, or a pharmaceutical composition thereof, and (b) instructions describing the administration of the dsDNA epitope, or a pharmaceutical composition thereof, to an individual to stabilize or improve the health-related quality of life of the individual.

In another embodiment, the invention provides a kit comprising (a) a dsDNA epitope, or a pharmaceutical composition thereof, and (b) instructions describing the selection of an individual suitable for receiving treatment by administration of a dsDNA epitope, or a pharmaceutical composition thereof, based on the low health-related quality of life of the individual.

In an additional aspect, the invention provides a kit comprising a dsDNA epitope which, as described herein, specifically binds to an anti-dsDNA antibody (or a pharmaceutical composition of the dsDNA epitope), and instructions comprising a description of the administration of the dsDNA epitope to an individual to stabilize or improve the health-related quality of life in the individual.

In still another aspect, the invention provides a kit comprising a dsDNA epitope which, as described herein, specifically binds to an anti-dsDNA antibody (or a pharmaceutical composition of the dsDNA epitope), and instructions comprising a description of the selection of an individual suitable for receiving treatment by administration of the dsDNA epitope based on the low health-related quality of life of the individual.

In another embodiment, the invention provides a kit comprising (a) a mimetic or an analog of a dsDNA epitope (e.g., an NR2 receptor epitope), or a pharmaceutical composition thereof, and (b) instructions for describing the administration of the mimetic or analog, or pharmaceutical composition thereof, to an individual to stabilize or improve the health-related quality of life of the individual.

In still another embodiment, the invention provides a kit comprising (a) a mimetic or an analog of a dsDNA epitope (e.g., an NR2 receptor epitope), or a pharmaceutical composition thereof, and (b) instructions describing the selection of an individual suitable for receiving treatment by administration of the mimetic or analog, or a pharmaceutical composition thereof, based on the low health-related quality of life of the individual.

In related aspects, the invention provides articles of manufacture that comprise the contents of the kits described above. For instance, in one additional aspect, the invention provides an article of manufacture comprising a dsDNA epitope which specifically binds to an anti-dsDNA antibody, and instructions comprising a description of the administration of the dsDNA epitope to an individual to stabilize or improve the health-related quality of life in the individual. In still another aspect, the invention provides an article of manufacture comprising a dsDNA epitope which specifically binds to an anti-dsDNA antibody, and instructions comprising a description of the selection of an individual suitable for receiving treatment by administration of the dsDNA epitope based on the low health-related quality of life of the individual.

In other embodiments, the invention provides compositions (described herein) for use in any of the methods described herein, whether in the context of use as a medicament and/or use for manufacture of a medicament.

### EXAMPLES

### Example 1

### SLE Patient Population Treated with LJP 394 (Phase II/III, 90-05)

A study was conducted in patients who met American College of Rheumatology criteria for the diagnosis of SLE, had a previous episode of SLE renal disease within four years, and had elevated anti-dsDNA≧15 IU/mL by the Farr assay at time of enrollment (Tan E M, et al. (1982) Arthritis Rheum 25:1271-7). Patients were excluded if they had evidence of a renal flare within three months of screening; were receiving prednisone or prednisone equivalent>20 mg/day, azathioprine>200 mg/day, methotrexate>25 mg/wk and/or cyclophosphamide at any dose within three months of screening; or a serum creatinine level≧2.5 mg/dL. The study was conducted in the US and Europe according to Good Clinical Practices and all patients provided voluntary informed consent.

A pharmacodynamic assay has been developed to measure the binding affinity of patients' anti-dsDNA antibodies to LJP 394 (Sem D S, et al. (1999) Arch Biochem Biophys 372:62-8; McNeeley P A, et al. (2001) Lupus 10:526-532). The assay measures the binding of the total serum immunoglobulin G [IgG] fraction to the dsDNA epitope on LJP 394. Binding of IgG to the LJP 394 epitope is measured using surface plasmon resonance and the concentration required to reach half maximal binding is determined. This concentration defines the apparent Kd′ of the binding interaction and reflects the titer-weighted average affinity of the patient's IgG fraction for the LJP 394 epitope. Using this assay, patients were segregated into “high affinity” and “low affinity” subgroups. The segregation value was selected by comparing the affinity measured before exposure to LJP 394 with that following 16 weekly treatments with LJP 394 100 mg or placebo. The high-affinity (HA) population was defined as those patients with antibody binding affinities [Kd′]≦0.8 mg/mL pre-treatment.

### Example 2

### Study Design for the Treatment of SLE Patients with LJP 394 (Phase II/III, 90-05)

In this double-blind, randomized controlled, multicenter trial, intravenously administered LJP 394 was compared with placebo in SLE patients with prior renal involvement. Patients were randomized to receive LJP 394 100 mg as a 2 ml bolus intravenous injection on a weekly basis or placebo for 76 weeks. After initiation of the trial, the protocol was amended to include 8 week off treatment periods alternating with 12 weekly treatments with 50 mg (1 ml bolus injection) LJP 394 or placebo. The first 16 weeks, when patients received 100 mg LJP 394 or placebo weekly, was considered the ‘induction period’, followed by ‘maintenance,’ when patients alternated 8 off and 12 weeks on treatment. The 20-week cycles were to be repeated three times for a total of 60 weeks.

The primary endpoint was the time to a documented renal flare, defined as reproducible increases from baseline in 24-hr proteinuria or serum creatinine>20% or at least 0.3 mg/dL, whichever was greater, accompanied by proteinuria, hematuria and/or red cell casts; or new onset or reproducible increase in hematuria accompanied by either an increase in 24-hr proteinuria or new red cell casts. Secondary outcome measures included the number of renal flares, time to institution of therapy with high dose corticosteroids (HDCC) and/or cyclophosphamide, incidence of treatment with HDCC and/or cyclophosphamide, and number of major SLE flares. HDCC was defined as an increase in oral, intravenous or intramuscular prednisone (or prednisone equivalent) greater than or equal to 15 mg per day from baseline to a dose greater than 20 mg per day for more than two days or any dose exceeding 200 mg in a single day. Topical, intra-articular, intra-lesional, or intra-ocular corticosteroid administration was excluded.

The trial was discontinued after an interim analysis suggested that the time to renal flare between treatment groups was unlikely to reach statistical significance in the intent-to-treat population. A subsequent analysis of the high affinity population, however, demonstrated a significant reduction in the time to and number of renal flares with active treatment compared with placebo.

### Example 3

### SF-36 Assessment of SLE Patients Treated with LJP394

As a secondary objective, the SF-36 was included in the protocol for analysis, on an exploratory basis, to evaluate and interpret changes in health-related quality of life with active treatment compared with placebo. An additional secondary objective was to evaluate changes in health-related quality of life before and after a documented renal flare, with or without prior treatment with HDCC. The SF-36 was administered at baseline and weeks 16, 24, 36, 44, 56, 64, 76, or at early treatment withdrawal. All patients with a baseline and at least one post treatment health-related quality of life assessment were included in the analyses. The eight SF-36 domains were scored according to the standard scoring rules identified in the SF-36 Health Survey Manual & Interpretation Guide (Ware J E, et al. (1993) SF-36 Health Survey: Manual and Interpretation Guide, The Health Institute, Boston, Mass.: Nimrod Press).

### Example 4

### Statistical Analysis

Statistical analyses were based on data following 16 weeks of treatment (induction phase) and over the entire protocol period. Missing observations and dropouts were replaced by the “last observation carried forward” (LOCF) approach. Specific analyses performed are shown in Table 1, below.

TABLE 1
Induction Treatment:
Change in mean scores, baseline to end of induction, for all
patients in the population under investigation [ITT population]
Change in mean scores, baseline to end of induction, for the high
affinity subpopulation [HA population]
Change in mean scores, baseline to end of induction, for all
patients in the population under investigation, excluding those
subjects who experienced a flare [ITT - flare]
Change in mean scores, baseline to end of induction, for all
patients in the high affinity population, excluding those subjects
who experienced a flare [HA - flare]
Change in mean scores, baseline to end of induction, for all
patients in the population under investigation, excluding those
subjects prescribed HDCC [ITT - HDCC]
Changes in HRQOL with Documented Renal Flares:
Patients with a documented renal flare: Change in mean scores
before and after a flare [n = 37]
Patients with a documented renal flare, excluding those receiving
HDCC prior to the last SF-36 completed before the flare: Change in
mean scores before and after a flare [n = 32]

Questionnaire results were evaluated for internal response consistency using the Response Consistency Index (RCI) as described by The Health Institute (Internal Memo (December 1994) The SF-36 Health Survey Response Consistency Index, The Health Institute, New England Medical Center, Boston, Mass.).

The presence or absence of floor or ceiling effects in domains of SF-36 was assessed. A floor effect corresponds to a high percentage of patients with the lowest possible score at baseline. A ceiling effect indicates that a high percentage of patients report the highest possible score at baseline. Ceiling effects are important to consider when interpreting data because improvement will not be readily detected if a large proportion of the population have maximal values at baseline.

Descriptive statistics were performed on each subgroup and exploratory chi-square analyses were performed on the entire ITT population for first 16 weeks only. Unless otherwise indicated, differences between treatment groups were not found to be statistically significant. An additional analysis was conducted for the percentage of patients whose change from baseline met or exceeded proposed values for MCID (5 points for individual domains and 2.5 for PCS and MCS summary scores).

### Example 5

### Baseline Characteristics of SLE Patients Treated with LJP 394

Of 230 patients randomized to receive treatment (114 LJP 394, 116 placebo), 214 were treated in North America and were included in the ITT analysis for time to renal flare. The remaining 16 patients treated in Europe, none of whom completed the induction phase of treatment, received a mean of 2.7 doses (median 3.0 doses, range 1 to 4 doses) and no renal flares were reported. These 16 patients are not included in this analysis. Pretreatment serum samples were available for affinity analyses in 213 of the 214 North American patients. One hundred and ninety patients (95 LJP 394, 95 placebo; 89% and 90%, respectively) had baseline and at least 1 post-baseline SF-36 assessments and are included in the ITT health-related quality of life analyses.

Baseline scores indicated decrements in all domains of SF-36 compared with age and gender matched normative values, with the exception of the Mental Health Index (see FIG. 1). The largest decrements were evident in the Physical Functioning, Role Physical and General Health Perceptions domains. Although 21 patients enrolled at a Mexican site (11 LJP 394 [1 male, 10 female]; 10 placebo [2 male, 9 female]), the small numbers of these patients in each subgroup are not expected to affect comparisons with US norms.

Baseline domain scores were comparable between treatment groups, with the exception of Role Emotional scores. The role emotional score was higher in placebo than in the active treatment group (80.7 vs 69.1) (see FIG. 1). Despite this difference, baseline MCS and PCS summary scores were comparable between treatment groups. MCS scores (active 48.9; placebo 50.8) approximated the US normative values (50 points) at baseline. Baseline PCS scores (active 41.0; placebo 38.4), however, were a full standard deviation below US norms, indicating substantial physical limitations in these patients.

Potential ceiling effects (i.e., highest possible score) in patients receiving active treatment included Role Physical (42%), Role Emotional (63%) and Social Functioning (38%); in placebo: Role Physical (45%), Role Emotional (70%) and Social Functioning (36%). These ceiling effects would make it more difficult to demonstrate large increases in these three domains, but are not meaningfully different between treatment groups. There were no floor effects, and therefore little concern regarding the ability to detect deterioration.

Decrements in SF-36 domains observed in this patient population compared with U.S. norms at baseline are similar to other reports in SLE patients and confirm that the effects of this disease on health-related quality of life are significant and differ from other chronic diseases, including rheumatoid arthritis. These results are consistent with those published by Gilboe et at, and DaCosta et al. who reported significant differences in all SF 36 domains, with the exception of role emotional, when comparing SLE patients with age and gender matched populations (Gilboe I, et al. (1999) Journ Rheum 26:1694-1700, DaCosta D, et al. (2000) Journ Rhem 27:365-372). Vu and Escalante also reported reduced scores in all SF-36 domains in patients with SLE, noting that those with end-stage renal disease had higher mental health scores, attributing this to withdrawal of treatment with high dose corticosteroids and/or immunosuppressives (Vu T, et al. (1999) Journ Rheum 26:2595-2601).

### Example 6

### Mean Chances in the SF-36 Domain Scores of the ITT Population

In the intent to treat (ITT) population, mean changes in domain scores from baseline to Week 16 following induction treatment were similar between active and placebo groups except for Role Emotional, Social Functioning and Role Physical (see FIG. 2). Role Emotional scores increased 7.7 points in the active treatment group, compared with a decrease of 8.1 points with placebo, resulting in a statistically significant difference in change scores of 15.8, favoring LJP 394 (p=0.0039). This 7.7 point improvement from baseline would also be considered clinically meaningful, based on proposed MCID values of 5 to 10 points. In patients receiving LJP 394, 24.2% reported improvements in Role Emotional scores greater than the MCID compared with 10.5% in the placebo group.

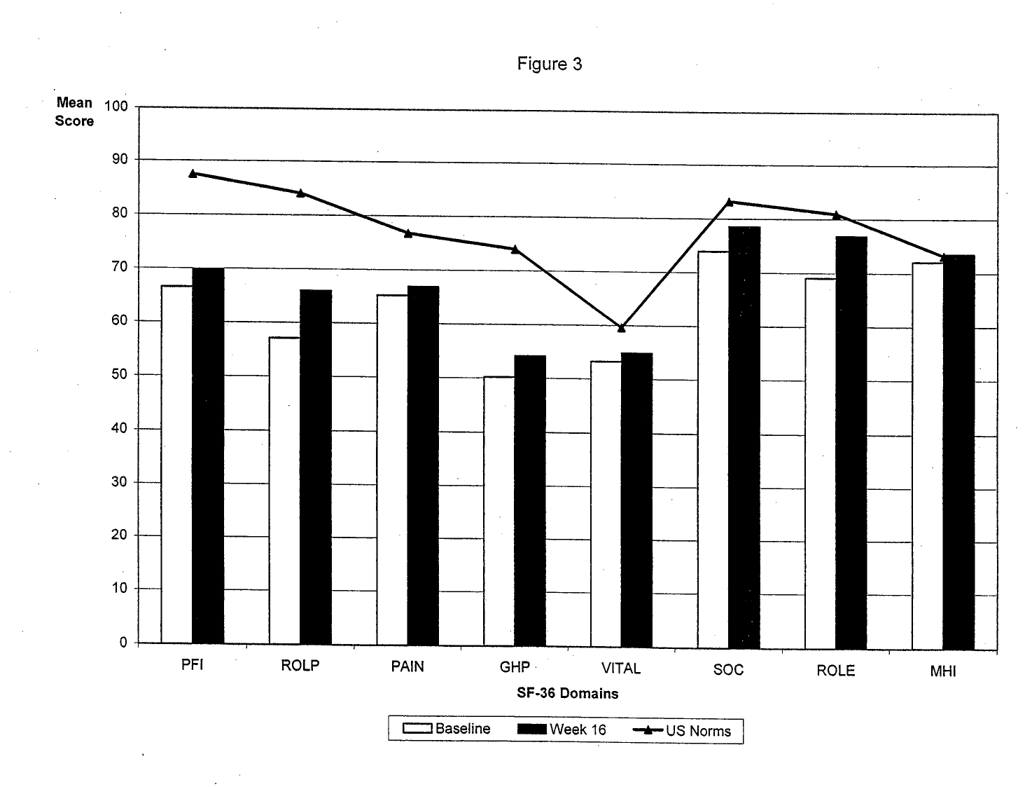

Improvements in Role Emotional and Social Functioning domains with active treatment are reflected in the improvement in MCS with active treatment, as compared with placebo. Mean changes from baseline to Week 16 in the physical (PCS) and mental component summary (MCS) scores of the SF-36 are shown in Table 2, below. This change of 2.1 numerically approaches proposed values for MCID for SF-36 summary scores. Improvement in Role Physical in the active treatment group (9.0 points) may also be considered clinically meaningful, although the magnitude of change compared with that seen with placebo was not great (5.3 points). Also of note, Social Functioning increased 4.6 points with active treatment compared with a minimal decrease with placebo (−0.1 points). Week 16 scores approached age and gender matched US norms for the 4 domains positively weighted in scoring the MCS score (Vitality, Social Functioning, Role Emotional and Mental Health), while domains positively weighted in the physical component summary score (PCS), despite some improvement, remained below normative values (see FIG. 3).

TABLE 2

| | LJP 394 | Placebo |
|---|---|---|
| SF-36 PCS | | |
| N | 94* | 93* |
| Baseline Mean | 41.03 | 39.41 |
| Mean Change | 1.76 | 2.27 |
| Mean % Change | 6.70% | 8.23% |
| SF-36 MCS | | |
| N | 94 | 93 |
| Baseline Mean | 48.91 | 50.73 |
| Mean Change | 1.29 | −0.83 |
| Mean % Change | 5.53% | −0.63% |

*Includes patients for whom both baseline and Week 16 evaluations were available.

During the induction phase and continuing through the second cycle of treatment, scores in all domains of SF-36 approached US norms in the active treatment group. It is interesting to observe that these improvements were reflected in improvements in MCS and, to a lesser degree, PCS summary scores. DeCosta et al. note, as have others, that MCS scores can be inflated when domains with negative scoring coefficients, such as Physical Functioning, Role Physical, and Bodily Pain, are significantly affected by a physical condition (Simon G E, et al. (1998) Med Care 36:567-572). Thumboo et al. have also discussed limitations in the summary scores, citing evidence that while PCS and MCS scores can demonstrate physical and mental health cross-sectionally, they are not as sensitive to changes in health related quality of life as are individual SF-36 domains (Thumboo J, et al. (2000) Journal Rheum 27:1414-1420). The authors conclude that the mean individual domain scores are preferable for studying prospective data. They also note that mental health status is negatively affected by corticosteroid and immunosuppressive therapy, as cited by Vu and Escalante. In the study reported here, prescription of high dose corticosteroids and/or immunosuppressive agents did not appear to acutely affect observed changes in SF-36 domain scores, as the results from a subgroup of patients who had not received such therapy demonstrated improvements similar to the entire population immediately following a flare. However, when those receiving high dose corticosteroids and/or immunosuppressive agents were excluded from the ITT analysis, improvements with active therapy were more evident.

### Example 7

### Mean Changes in the SF-36 Domain Scores of the High Affinity Population

The high affinity (HA) population included 82 active and 86 placebo-treated patients, representing 90% and 86% of the ITT health-related quality of life population. Baseline scores for all SF-36 domains and component summary scores in this HA population were similar to the ITT population and between active and placebo treatment groups.

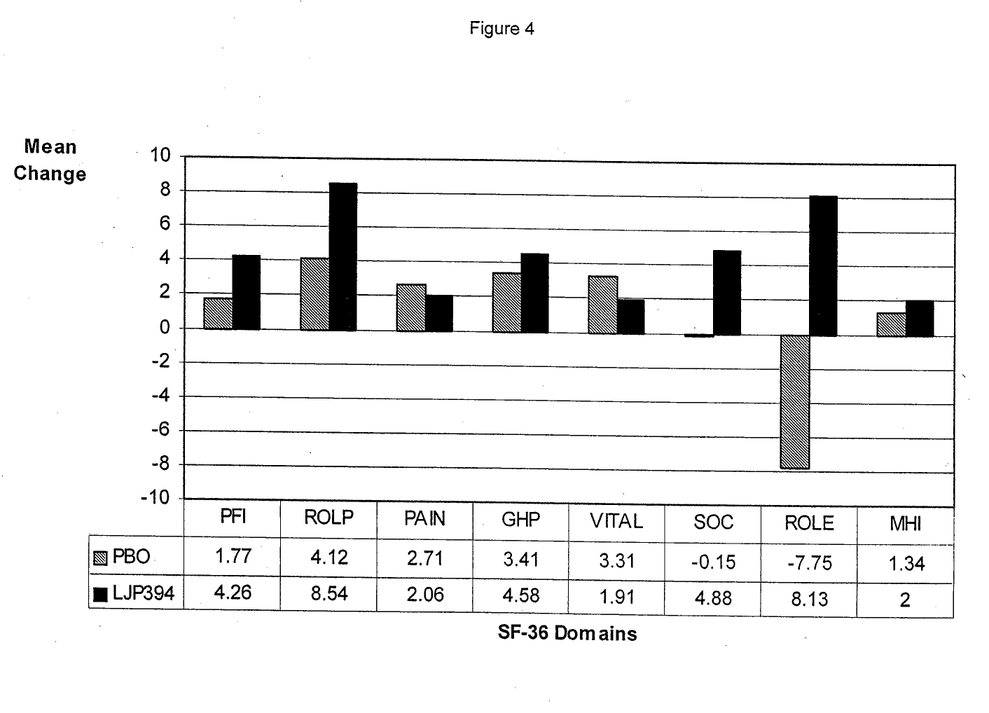

Mean changes from baseline in the HA population parallel changes seen with active treatment in the ITT population (see FIG. 4). Differences in change scores between active and placebo in Role Emotional and Role Physical domains were replicated in this HA population: 15.9 compared with 15.8 in the ITT population and 8.5 compared with 8.9 points, respectively. The magnitude of differences between active and placebo on Role Physical, however, was greater in the high affinity population (4.4 points) than in the ITT population. Changes in Social Functioning also follow these trends, with improvement of 4.9 points with active treatment and minimal decline with placebo of −0.2 points. The magnitude of these differences between treatments (5.0) may be considered clinically meaningful.

Due to high baseline values, only small changes in MCS scores were expected in either treatment group. Nonetheless, the difference between change scores is 2.2 points and approaches a clinically meaningful difference of 2.5. This trend is likely driven by improvements in Role Emotional and Social Functioning domains with active treatment. Mean changes from baseline to Week 16 in the high affinity population for PCS and MCS scores of the SF-36 are shown in Table 3, below.

TABLE 3

| | LJP 394 | Placebo |
|---|---|---|
| SF-36 PCS | | |
| N | 81* | 84* |
| Baseline Mean | 41.13 | 40.19 |
| Mean Change | 1.88 | 1.83 |
| Mean % Change | 7.17% | 7.18% |
| SF-36 MCS | | |
| N | 81 | 84 |
| Baseline Mean | 48.21 | 51.11 |
| Mean Change | 1.49 | −0.72 |
| Mean % Change | 6.10% | −0.33% |

*Includes patients for whom both baseline and Week 16 evaluations were available.

### Example 8

### Other Subgroup Analyses of SLE Patients Treated with LJP 394

The same analyses were performed in the ITT and HA populations excluding those patients with documented renal flares. Baseline SF-36 domain and component summary scores in these groups were similar to the entire ITT and high affinity populations and similar between treatment groups, again favoring placebo in Role Emotional. Excluding patients with documented renal flare, the ITT population numbered 77 active and 75 placebo and the HA population 75 active and 67 placebo treated patients.

Mean changes from baseline in these groups confirmed those changes observed between treatment groups in the entire ITT and HA populations.

### Example 9

### Longitudinal Changes Among SF-36 Scores (Phase II/III Study)

Role Emotional and MCS scores were also evaluated from a longitudinal perspective (comparing all patients in the drug-treated population to all patients in the placebo-treated population regardless of the occurrence of renal flares). Available data were plotted to review any trends or changes in domains of SF-36 over the entire treatment period, despite obvious attrition of patients assessed over time (see FIG. 5). These plots were derived using only available data, i.e., no values were imputed via LOCF or any other method. Although Role Emotional and MCS scores at baseline were higher in the placebo group, after the 16 week “induction” phase, marked improvement was evident with active treatment compared with a deterioration of the scores in the placebo group. However, by week 36 of treatment, Role Emotional and MCS scores were comparable between the two groups and mirrored each other until endpoint. This was due in part to attrition, as data were available for only a small number of patients on or after week 36. Differences between treatment groups are also likely to have decreased after the induction phase due to the intermittent treatment and use of lower doses of LJP 394. At study end, SF-36 data were available for analysis in 46 patients receiving placebo and 45 active treatment.

Changes in dsDNA antibodies and C3 at each assessment point are shown in FIG. 6 for the ITT population. Again, differences between the active treatment and placebo treatment groups are believed to have decreased after the end of treatment cycle 2 in part due to intermittent treatment and use of lower doses of LJP 394 as well as attrition of patients. During induction and until the second ‘off treatment’ phase plotting change scores in Role Emotional domain are similar to those for decreases in anti-dsDNA titers and increases in complement 3 levels in the active treatment but not in placebo group (see FIG. 6).

In patients receiving active treatment, longitudinal changes in Role Emotional (and MCS) scores appeared to correlate with decreases in anti-dsDNA titers. The striking effect on Role Emotional scores perhaps reflect an effect of these antibodies on CNS function, separate from their effect on renal disease, as recently suggested by DeGiorgio et al. (Kozora E, et al. (1997) Arth Rheum 39:2035-45, DeGiorgio L A (2001) Nature Medicine 7:1189-94).

### Example 10

### Changes in Health-Related Quality of Life Following Renal Flare

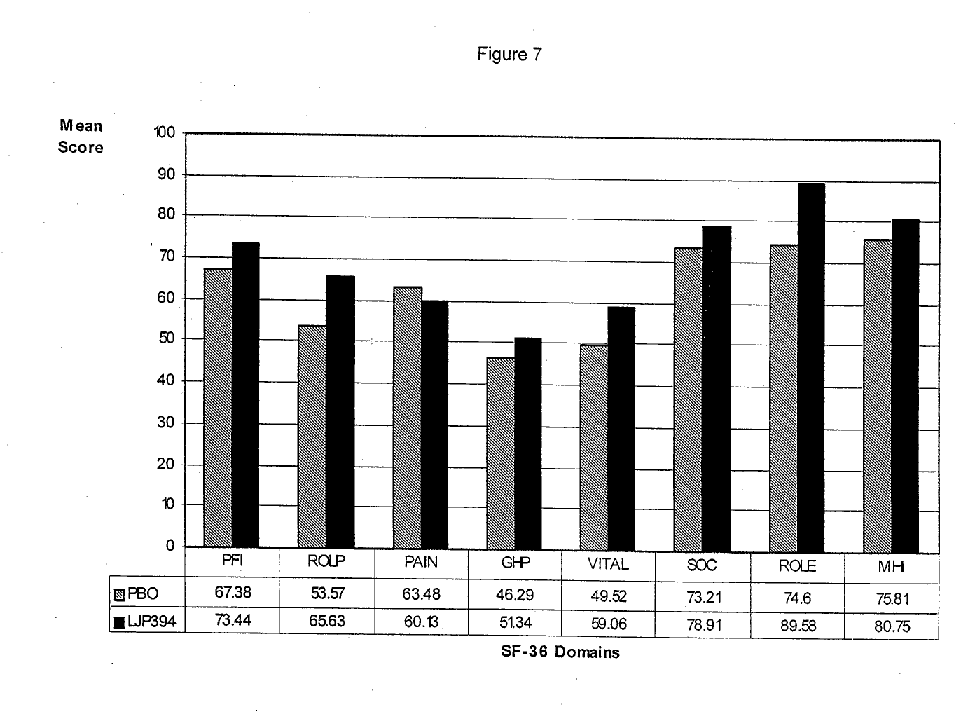

SF-36 assessments before and after a documented renal flare were compared between treatment groups. Of the entire population of 42 patients with documented renal flares, 37 patients had SF-36 data preceding and following a renal flare and are included in this analysis. Despite similar scores at baseline (pre-treatment), patients receiving active treatment reported better scores than placebo in all domains except pain prior to renal flare, indicating a treatment effect even in those patients who subsequently developed worsening manifestations of SLE (see FIG. 7). Pre-flare scores again reflected a difference between treatment groups in Role Emotional (15.0 points) (see FIG. 7), as well as Vitality (9.5 points) domains and MCS (4.1 points) summary score, which meets or exceeds the proposed MCID value of 2.5, and approaches improvement of one-half of the standard deviation.

SF-36 results reported 0 to 16 weeks following a documented renal flare demonstrated patients receiving active treatment experienced sustained or improved health-related quality of life when compared with results reported 2 to 10 weeks prior to renal flare.

Differences in domain change scores between LJP 394 and placebo treated patients increased further post-flare (see FIG. 8). Role Emotional demonstrated a difference in scores of 37.7 points, favoring active treatment, which is very large and clinically meaningful. Differences in other domain change scores favored active treatment over placebo by 9.3 to 17.2 points, all of which meet or exceed proposed MCID values of 5-10 points and would be expected to be clinically meaningful. Domain scores before and after a documented renal flare were unchanged or improved with active treatment (with the exception of General Health Profile) including 12.3 points in Bodily Pain, 4.1 in Vitality, 2.3 in Social Functioning and 2.1 in Role Emotional compared with deteriorations in placebo (−3.6, −7.1, and −20.6, respectively).

To evaluate the possible effect of concomitant therapies before and following a documented renal flare, five patients (2 active, 3 placebo) receiving HDCC prior to the “pre-flare SF-36” assessment were excluded. There were 14 LJP 394 and 18 placebo-treated patients with SF-36 data from the time periods 2-10 weeks preceding and 0-16 weeks following a documented renal flare. Excluding these 5 patients who received HDCC, the differences in change scores between the active and placebo treatment groups were similar to the results from the previous analysis. Together, these data indicate that institution of high dose corticosteroid or cytotoxic therapy was not responsible for the reported changes in health-related quality of life in either group.

Unfortunately, attrition of patients past the second treatment cycle, as well as drug holidays and change in dosing, makes it difficult to assess changes in scores past this timepoint. However, pre-flare SF-36 domain scores in patients with documented renal flares support a benefit of continued active treatment, as SF-36 domain scores were improved from baseline in patients receiving LJP 394, and worsened in the placebo group. Importantly, improvements in Role Emotional and Social Functioning domain scores with active treatment were replicated in patients without a documented renal flare, when no change in disease manifestations or treatment were reported.

Another important observation was that patients receiving LJP 394 reported sustained or even improved health-related quality of life despite having a documented renal flare. This is most striking when compared with placebo treated patients who reported deterioration in all SF-36 domains following renal flare.

### Example 11

### Summary of Mean SF-36 Domain Scores Across Treatment Groups

Treatment with LJP 394 improved health-related quality of life during the induction phase of the trial, when disease manifestations of SLE and treatments were unchanged in most patients. This benefit was evident in the entire ITT as well as the ‘high affinity’ populations. Renal flares might be expected to affect health-related quality of life scores, but the improvements were unchanged when the 13 patients with documented renal flares during this time were excluded from the analyses. Weekly treatment with LJP 394 over 16 weeks appeared to have a true clinical benefit in health-related quality of life, as reflected by changes in Role Emotional, and to a lesser degree, Social Functioning scores, compared with deterioration in the placebo group. A summary of differences in change scores between treatment groups in Role Emotional and Social Functioning domains is shown in Table 4, below. These differences were observed despite the presence of potential ceiling effects at baseline for these domains.

TABLE 4

| | Role | Social | # of Patients |
|---|---|---|---|
| | Emotional | Functioning | (active/placebo) |
| ITT | 15.8 | 4.7 | 95/95 |
| High Affinity | 15.9 | 5.0 | 82/86 |
| ITT, no flares | 12.3 | 5.0 | 77/75 |
| High Affinity, | 13.2 | 5.5 | 75/67 |
| no flares | | | |
| ITT, no HDCC | 9.7 | 5.9 | 74/61 |

The reported changes in SF-36 scores demonstrated improvements in health-related quality of life in patients receiving active treatment compared with placebo.

### Example 12

### Study Design for the Treatment of SLE Patients with LJP 394 (Phase III)

Patients were treated with weekly doses of 100 mg of LJP 394 or with placebo. Patients were also permitted to receive certain other treatments including some but not all immunosuppressive drugs using a definition similar to Example 2. This randomized, double-blind, placebo-controlled study was conducted at more than 70 major medical centers in North America and Europe. Patients could remain in the study for up to 92 weeks.

The prospectively defined analysis groups were the intent-to-treat population and patients with impaired renal function. The intent-to-treat population was defined as patients with high-affinity antibodies to LJP 394. The patients with high-affinity antibodies to LJP 394 were those with a Kd′<0.8 mg/ml. Patients with impaired renal function were defined as having a serum creatinine level of 1.5 mg/dL to 3.5 mg/dL at baseline. In general, patients with impaired renal function are at greater risk of progressing to renal flare, kidney failure, and dialysis.

The primary endpoint was time to renal flare. A renal flare was defined as a significant, reproducible increase in serum creatinine, urine protein or blood in the urine as described in Example 2. The secondary endpoint was time to treatment with HDCC. HDCC was defined as any dose of cyclophosphamide or an increase in prednisone of 15 mg/day or higher resulting in a final dose greater than 20 mg/day.

Other prospectively defined secondary outcomes included time to Major SLE flare, treatment associated maintenance and/or improvement in health-related quality of life, decreases in antibodies to dsDNA and associated increases in complement C3 levels. A Major SLE flare was defined as the occurrence of any one of the following due to manifestations of active SLE: treatment with HDCC or initiation or increase in treatment with other immunosuppressive agents, including azathioprine, mycophenolate mofetil, methotrexate, cyclosporin and leflunomide; or hospitalization or death. This definition of Major SLE flare was designed to capture serious events where patients were treated for hospitalization or death could have preceded the occurrence of a documented renal flare.

Complement changes were evaluated by determining the mean change from baseline in the complement protein C3 that indicates overall complement consumption due to active inflammation. Antibody changes were evaluated by determining the mean percent change of antibodies to dsDNA from baseline. Patients' assessments of disease activity and health-related quality of life were measured on a regular basis as well as at the time of, and 30 days following, a documented renal flare.

### Example 13

### Determination of Anti-dsDNA Antibodies

Two studies (Phase II/III or 90-05, and Phase III or 90-09) were conducted as described in Examples 1-2 and 12. The Phase 3 trial enrolled 298 lupus patients with high-affinity antibodies to LJP394 who were treated for up to 22 months with either LJP394 or placebo. Patients completed the SF-36® assessment at entry, followed by three additional time points in the trial depending on how long they participated. Patients who had a renal flare completed the assessment once the renal flare was confirmed. The Phase II/III trial enrolled 198 lupus patients with high-affinity antibodies who were treated for up to 18 months. Patients selection, duration of treatment, assay type were in these studies are further detailed in Table 5. All laboratory values were determined at a central laboratory. Baseline anti-dsDNA antibody levels were calculated as the mean of the last 2 determinations prior to initial administration of the study drug. Baseline values for all other laboratory measures were determined immediately before administration of the study drug. The upper limit of normal for the anti-dsDNA antibody assay at the central laboratory was 5 IU/ml.

TABLE 5
Key trial design considerations

| | Phase II/III | Phase III |
|---|---|---|
| Patients enrolled | 189 (High-affinity) | 298 (High-affinity) |
| Protocol length | 18 month | 22 month |
| Average duration | 12.4 month, 12.7 month | 10.3 month, 11.4 month |
| (LJP 394, Placebo) | | |
| Assay type | Farr | Farr |
| Assay frequency | Weekly to monthly | Weekly to monthly |
| Assay QC | Central lab | Central lab |
| Positive anti- | 189 | 298 |
| dsDNA antibodies | | |
| at baseline | | |

### Example 14

### Effect of LJP394 on Anti-dsDNA Antibodies

The data from the Phase II/III and Phase III studies indicate that LJP 394 causes the intended pharmacological effect of a reduction of anti-dsDNA antibodies. Treatment with LJP 394 was associated with a statistically significant decrease in anti-dsDNA antibodies from baseline compared with placebo in both the Phase II/III and Phase III studies (p<0.0001 for both studies).

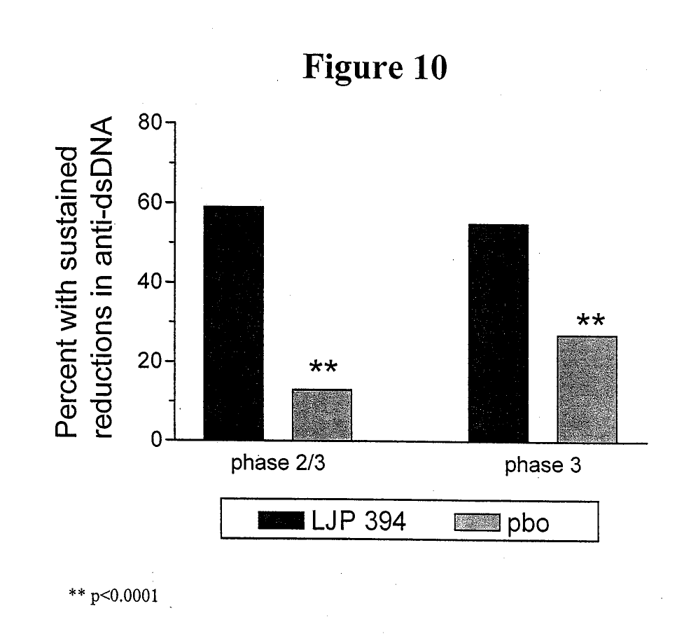

In addition, there was a higher frequency of patients with sustained reductions in anti-dsDNA antibodies in the LJP 394 treated group compared with placebo in the LJP 394-90-09 and LJP 394-90-05 trials as presented in FIG. 10 (p<0.0001 for both studies).

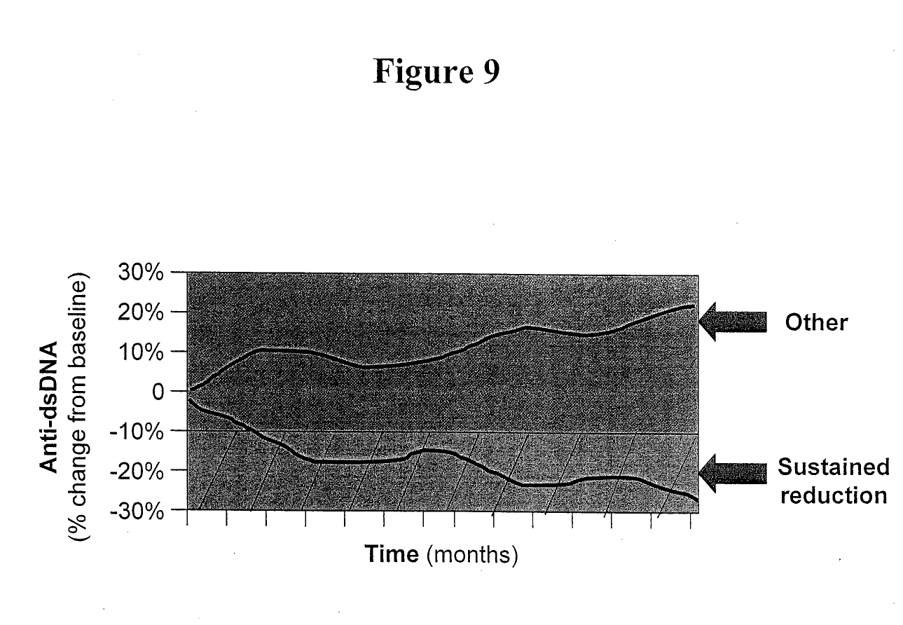

A patient was defined as having a sustained reduction in anti-dsDNA antibodies if they had an at least 10% reduction from baseline for at least ⅔ of all observed values prior to treatment with HDCC or drug (placebo or LJP 394). (Sustained reduction of 10% was based on the percent coefficient of variation of the Farr assay as reported in the assay labeling. At least ⅔ of observations were required to meet this criterion to assure the majority of values were represented for a patient.) All anti-dsDNA antibody values collected were used to determine if a patient met the criterion for sustained reduction. Anti-dsDNA antibody values subsequent to HDCC were considered treatment failures and these values were imputed to have a value equivalent to baseline. One possible example of this analysis is shown in FIG. 9. (FIG. 9 is a schematic for illustrative purposes only and does not reflect any particular set of actual data.) For a patient to be characterized as having “sustained reduction”, ⅔ of all anti-dsDNA antibody determinations would need to fall into the area marked “Sustained reduction” in the figure (the area shown with hatch marks).

Four times as many LJP 394-treated patients had sustained reductions compared with placebo-treated patients in the Phase III study. Twice as many LJP 394-treated patients had sustained reductions compared with placebo-treated patients in the Phase III study.

Several observations from the Phase II/III study and Phase III studies link changes in anti-dsDNA antibodies with complement (C3) and link both of these variables with renal flare. Changes in anti-dsDNA antibodies were inversely correlated with changes in levels of C3 in both the Phase II/III study and Phase III studies (p<0.0001 for both studies). Increases in anti-dsDNA antibodies correlated with renal flare (p<0.001 for both studies) and decreases in C3 tended to correlate with renal flare (p=0.02 for Phase III; p>0.05 Phase II).

### Example 15

### Patients with Sustained Reductions in Anti-dsDNA have Improved Health-Related Quality of Life (HRQOL) (Phase II/III and Phase III)

As was the case in the Phase II/III study, at the beginning of the Phase III study, the mean SF 36® scores for all patients were significantly lower in all domains compared with normal individuals in the U.S. of similar age and sex, indicating that these lupus patients' perception of their quality of life was poor.

Patients in the Phase II and Phase III studies were tested for levels of circulating anti-dsDNA antibodies in serum. Based on the levels of anti-dsDNA antibodies were segregated into two groups: sustained reduction group and other group. Patients that had sustained reduction were defined by having at least about 10% reduction below baseline in anti-dsDNA antibody for at least ⅔ of all observed values prior to treatment with HDCC or last (most recent) dose of LJP 394 or placebo (the percent CV (co-efficient of variance) for the Farr assay is about 10%). Patients in the other group were any patients that did not meet the above definition for sustained reduction. On average, the sustained reduction patients showed decreases of at least about 25% compared to baseline.

As described above in the detailed description of the invention, HRQOL is an assessment of a patient's sense of physical and mental well-being or how they feel. In the Phase II/III and Phase III studies, HRQOL was measured by a standard scoring instrument called the Medical Outcomes Study 36-Item Short Form (SF-36®). The SF-36® Health Survey is a standardized tool used in clinical research to measure a patient's assessment of quality of life outcomes related to disease and disease treatment. The Survey asked 36 questions related to how well a patient can perform day-to-day activities and how they feel emotionally. The answers were then reported as scores in eight domains. These domains are physical functioning, role-physical, bodily pain, general health, vitality, social functioning, role-emotional and mental health. SF-36® was measured in both Phase III and II/III.

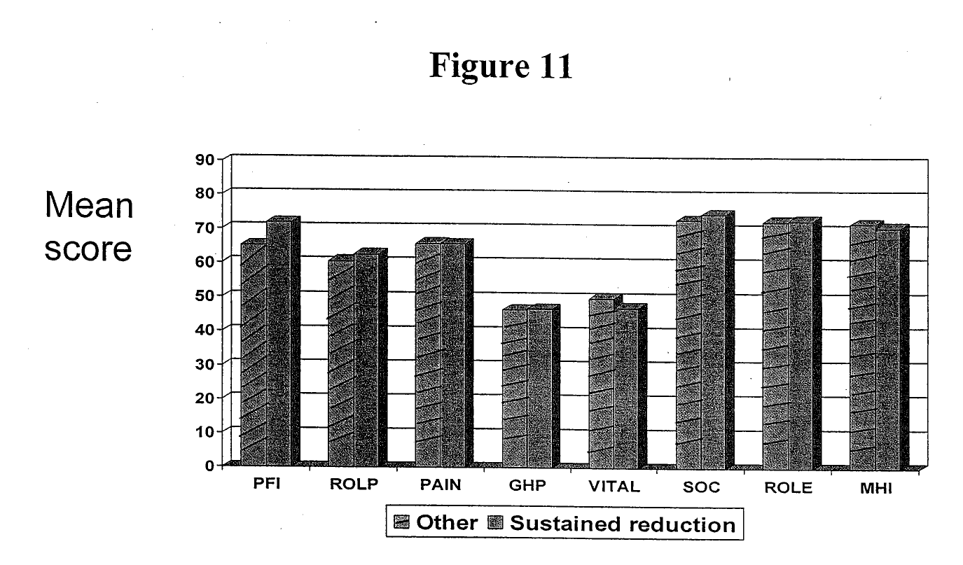

The baseline HRQOL domain scores were balanced for patients with sustained reductions. The comparable mean HRQOL domain scores of the sustained reduction group and the other group are shown in FIG. 11.

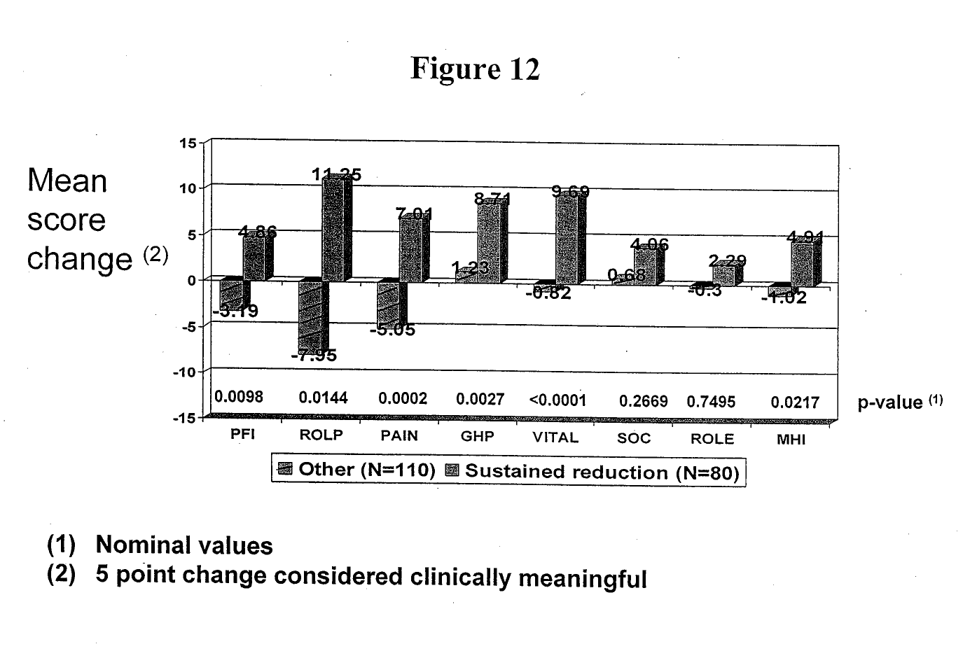

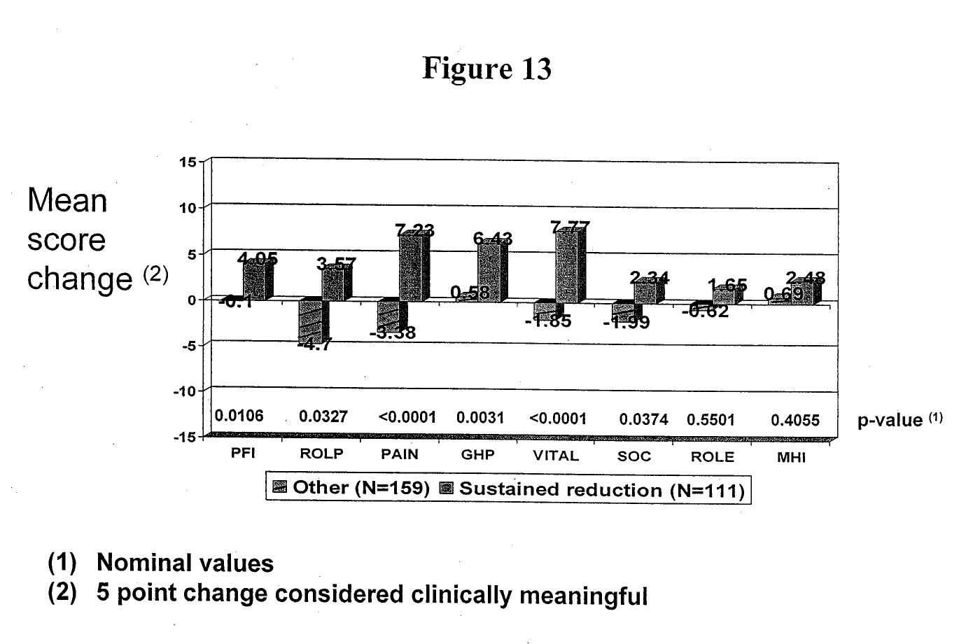

The SF-36® results indicated that patients in the phase III study with sustained reductions in antibodies to dsDNA showed statistically significant improvement at week 24 in the following domains: physical functioning (p=0.0106), role physical (p=0.0327), bodily pain (p<0.0001), general health perception (p=0.0031), vitality (p<0.0001) and social functioning (p=0.0374). Role emotional and mental health of the patient with sustained reductions also appeared to be improved relative to the others, although results were not statistically significant for these two domains. The results at week 24 are shown in FIG. 13. Similar results were seen at week 48, see FIG. 12 and Table 6. The p values are nominal and based on ANCOVA with baseline level as a covariate.

TABLE 6
SF 36 ® Scores for patients with sustained reductions at 48 weeks

| | Domain | | | | | | | |
|---|---|---|---|---|---|---|---|---|
| | PFI | ROLP | PAIN | GHP | VITAL | SOC | ROLE | MHI |
| Mean score | 4.9 | 11.3 | 7.0 | 8.7 | 9.7 | 4.1 | 2.3 | 4.9 |
| for patients | | | | | | | | |
| with | | | | | | | | |
| sustained | | | | | | | | |
| reductions | | | | | | | | |
| Mean score | −3.2 | −8.0 | −5.1 | 1.2 | −0.8 | 0.7 | −0.3 | −1.0 |
| for other | | | | | | | | |
| patients | | | | | | | | |
| P value | 0.0098 | 0.0144 | 0.0002 | 0.0027 | <0.0001 | ns | ns | 0.0217 |

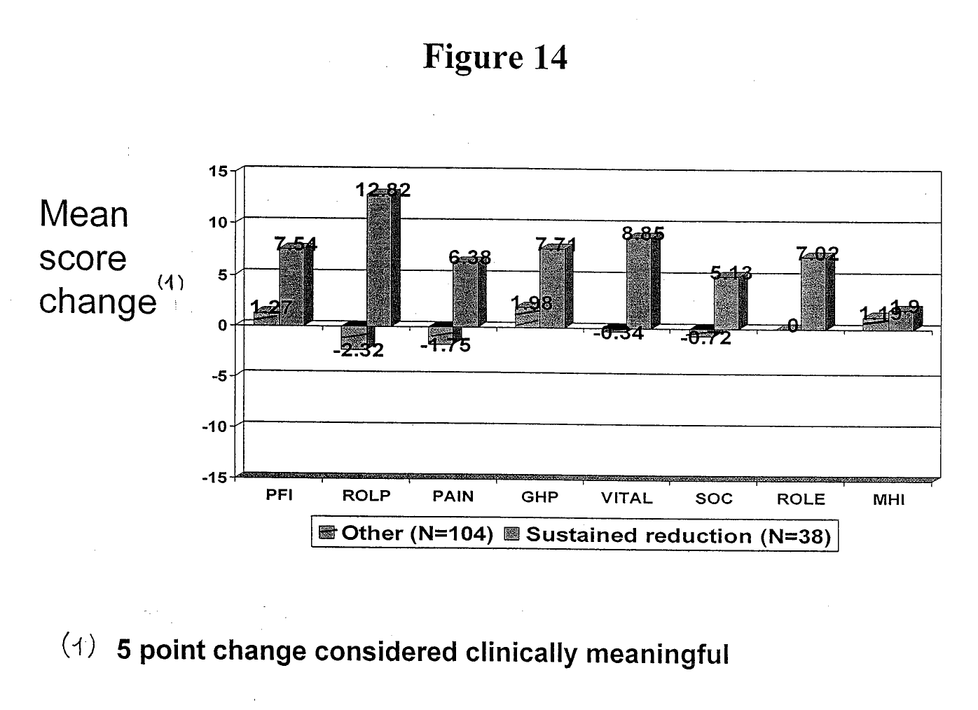

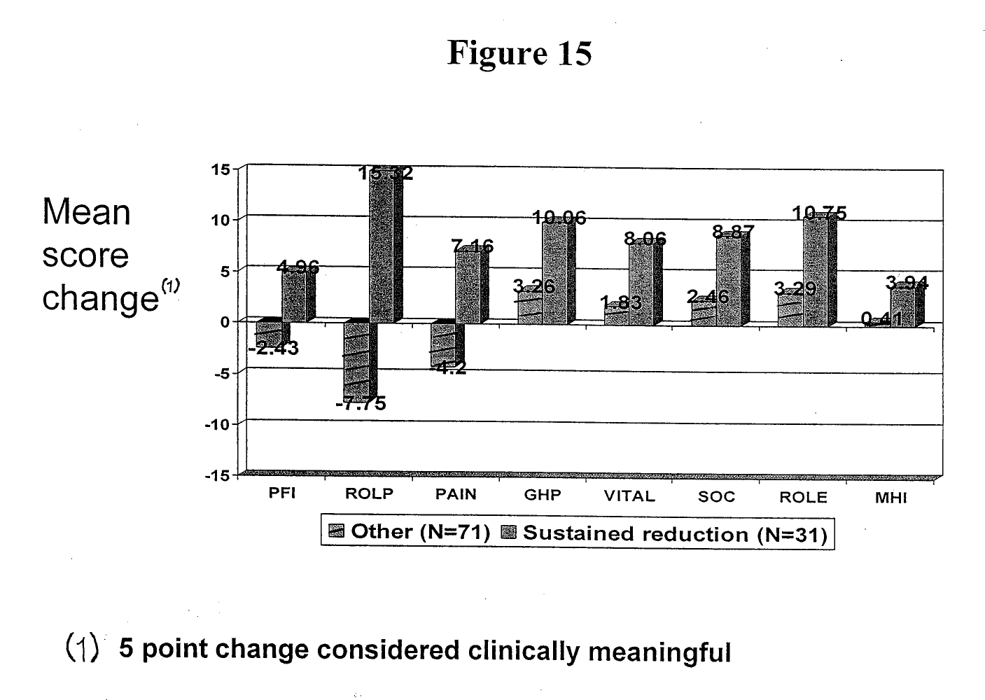

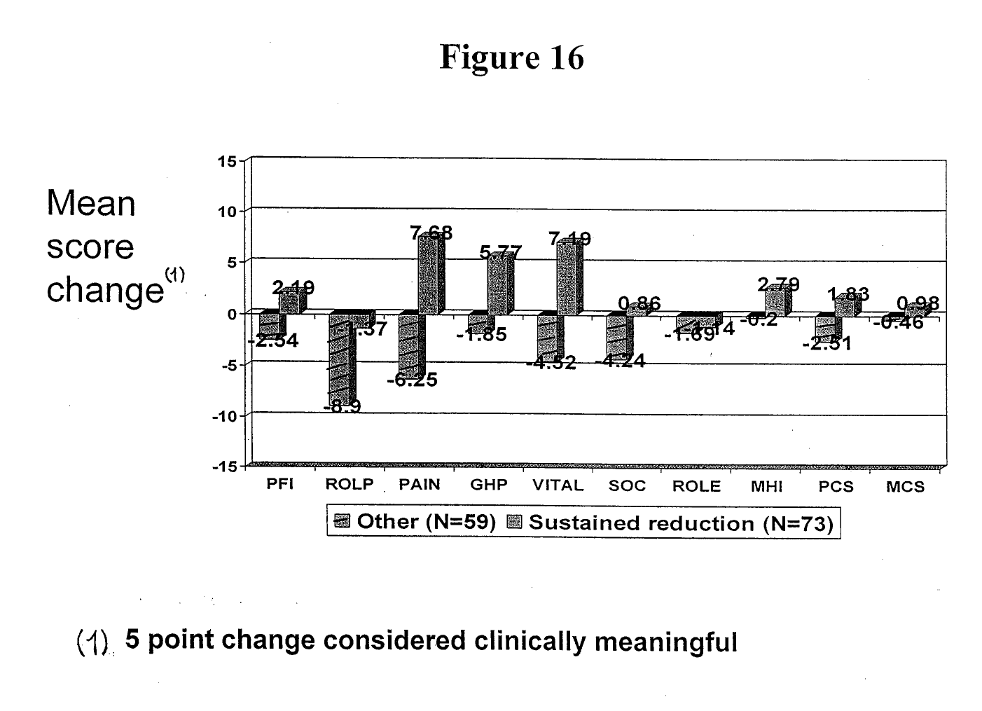

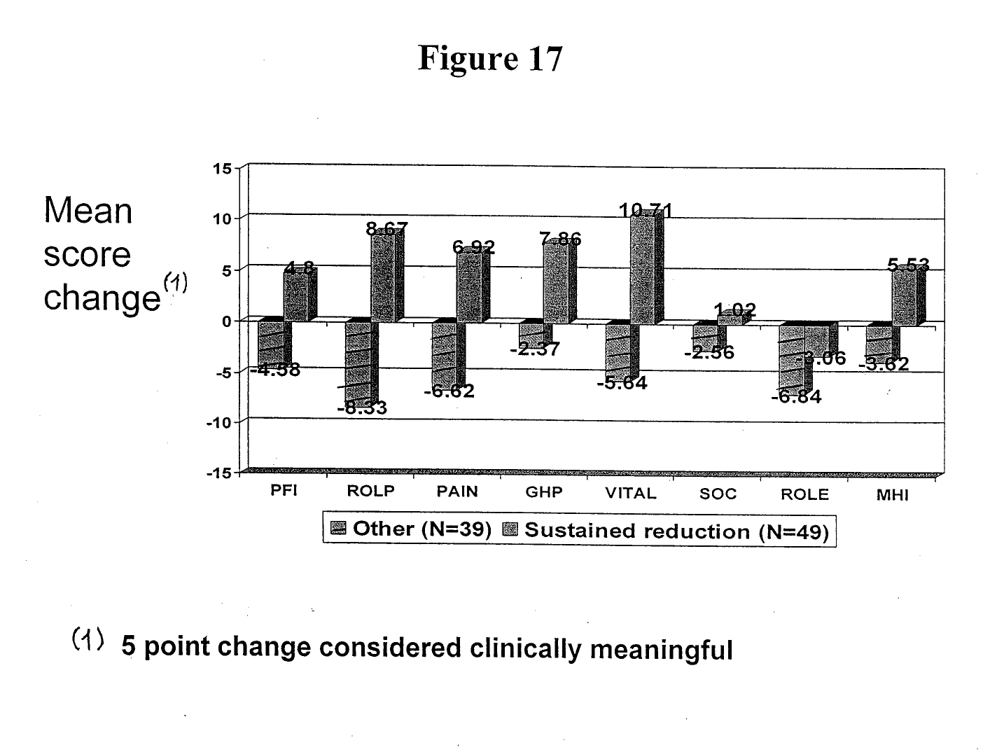

In addition, among the placebo-treated patients of the Phase III study, HRQOL domain scores favored patients with sustained reductions of anti-dsDNA antibodies following both 24 weekly treatments (FIG. 14) and 48 weekly treatments (FIG. 15). In addition, among LJP394-treated patients of the Phase III study, HRQOL domain scores favored patients with sustained reductions of anti-dsDNA following both 24 weekly treatments (FIG. 16) and 48 weekly treatments (FIG. 17).

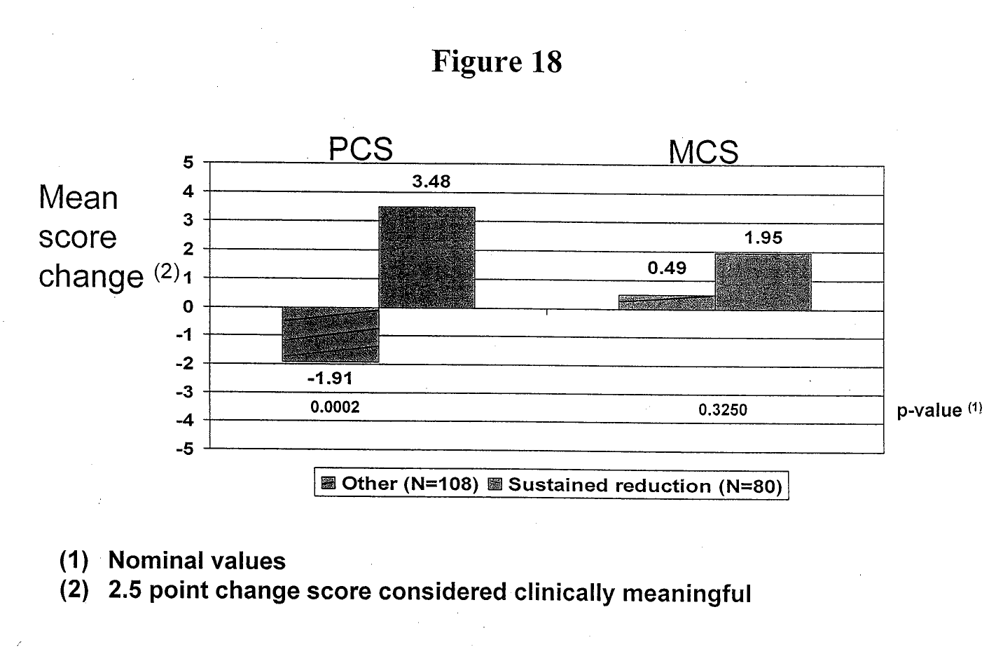

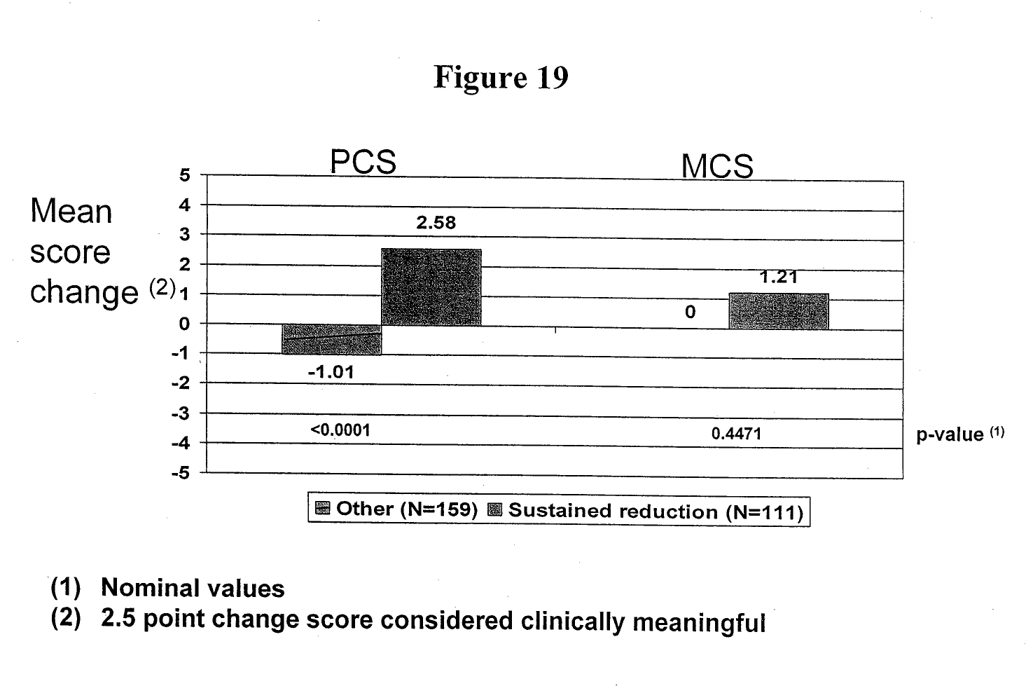

Following 48 weekly treatments, the mean change in the physical component summary score (PCS), a summary of the physical components from the eight SF 36′ domains, favored patients in the Phase III study with sustained reductions in antibodies to dsDNA (see FIG. 18). The mean change in PCS also favored patients in the Phase III study with sustained reduction in antibodies after 24 weeks (see FIG. 19). The change in mean PCS score was statistically significant at weeks 24 and 48 (p<0.001 for both time points). Mean score changes at week 24 for PCS were 2.58 and −1.01 for patients with sustained reduction and Other patients, respectively. Changes of 2.5 on the component summary scores are normally considered clinically meaningful. Similar results were seen at week 48. Statistical significance for the mental component summary score was not observed.

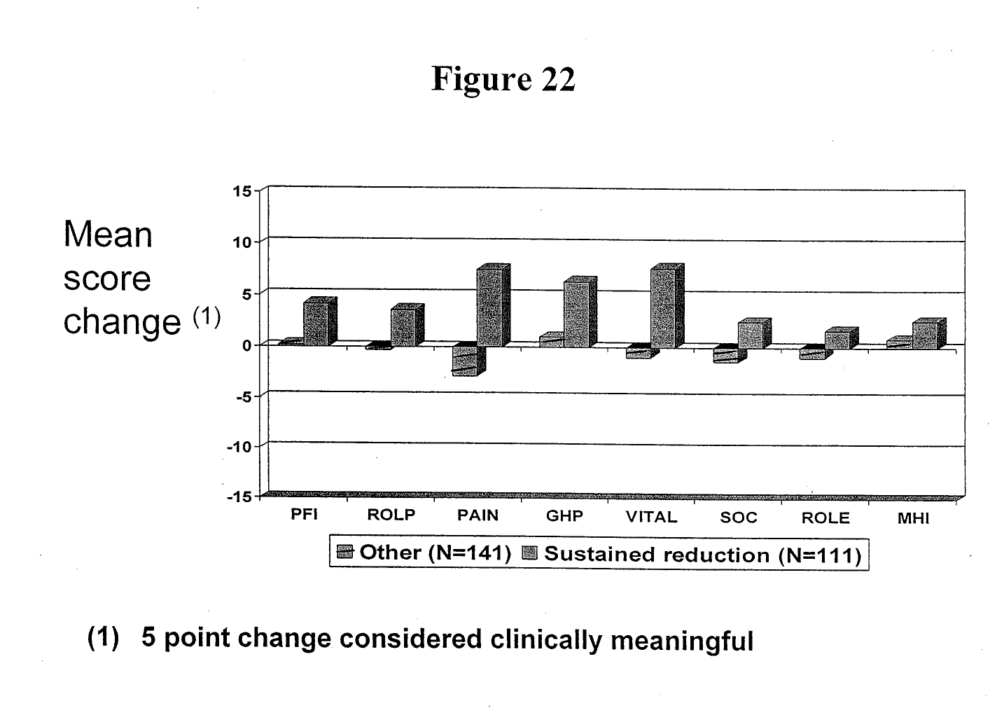

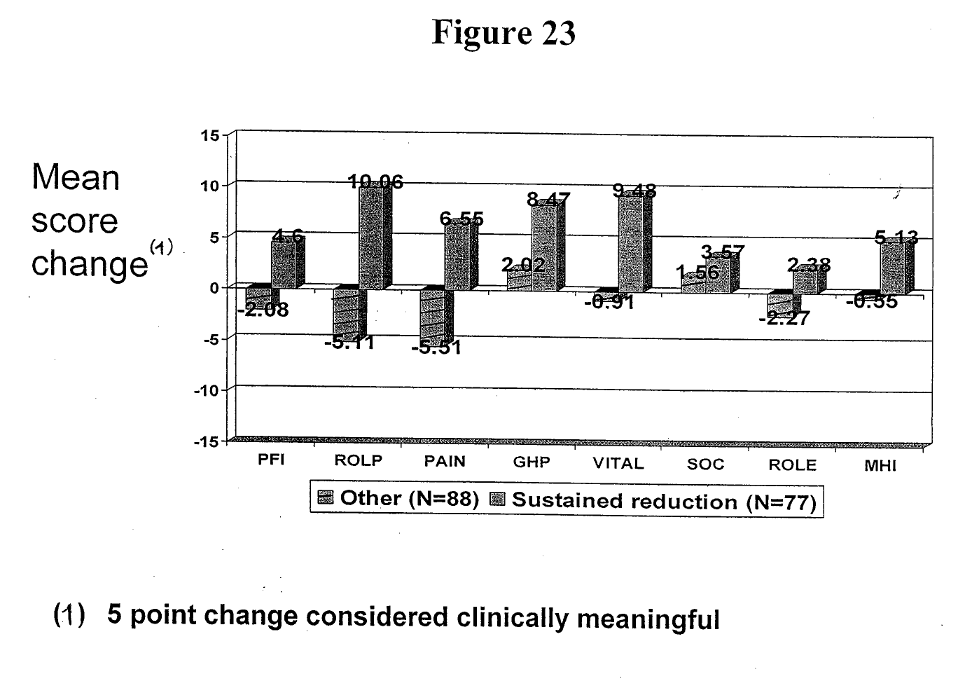

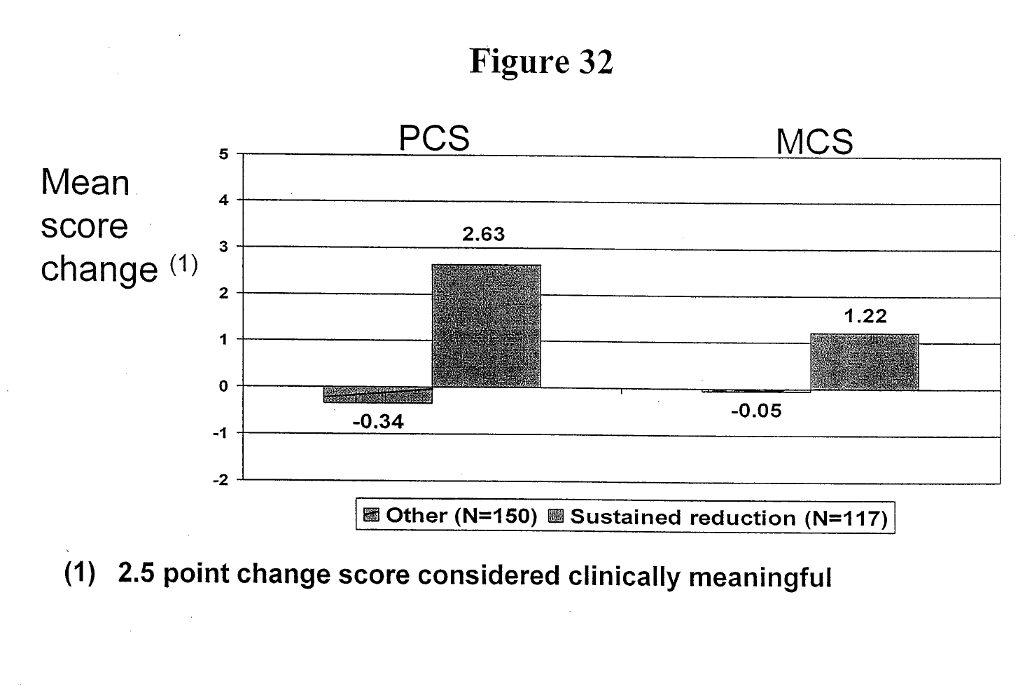

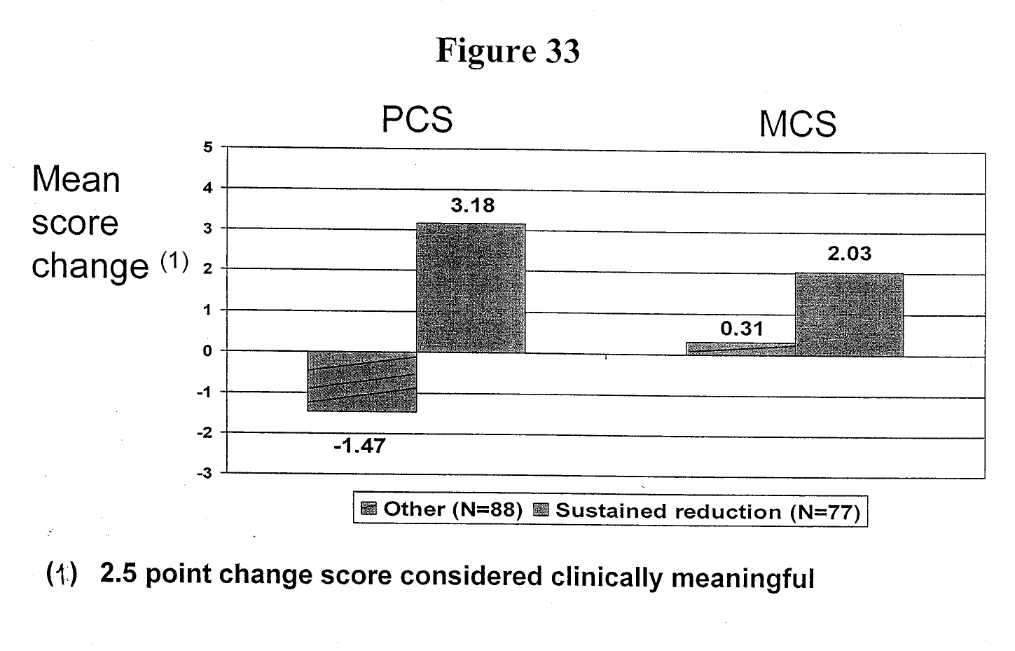

It should be noted that the HRQOL results for patients with sustained reductions in antibodies to dsDNA were similar at week 24 and 48 for domain scores and summary component scores after excluding patients that had a documented renal flare from the analysis, suggesting that patients with sustained reductions feel better beyond issues caused by a renal flare (see data in FIGS. 22, 23, 32, and 33).

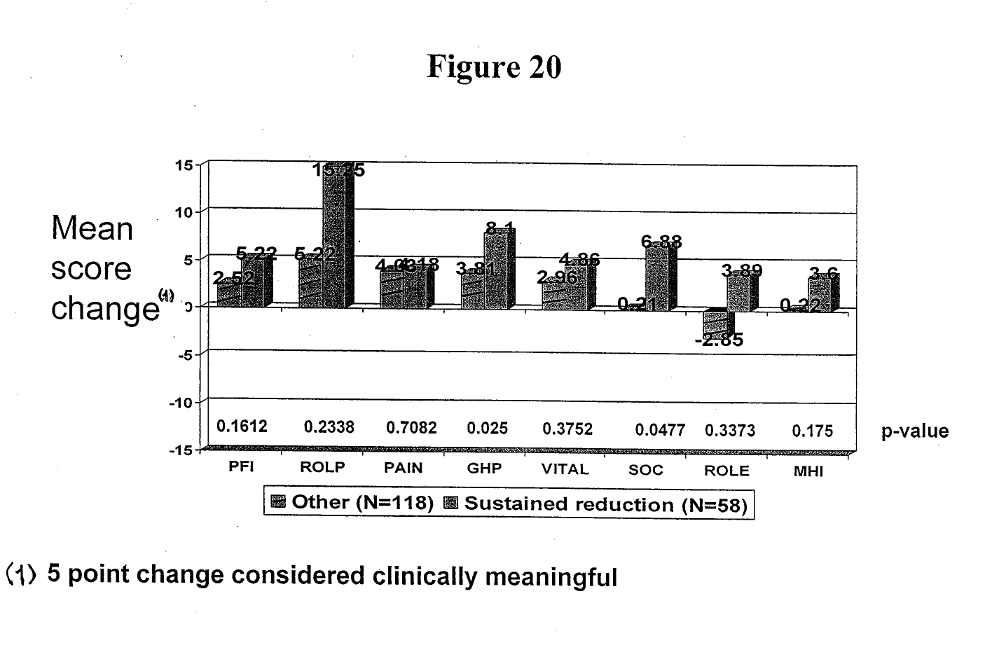

For patients with sustained reductions in antibodies to dsDNA at 16 week (n=118) in the Phase II/III study, all eight SF 36® domains were also improved when compared with patients that did not have sustained reductions (n=58), as measured by mean score changes within a domain (FIG. 20). Furthermore, two of the eight domain scores reached statistical significance even at this early time point when compared with patients that did not experience sustained reductions. Because of a change in dose-regimen after an 16-week induction period, week 16 was considered the most relevant analysis in this trial.

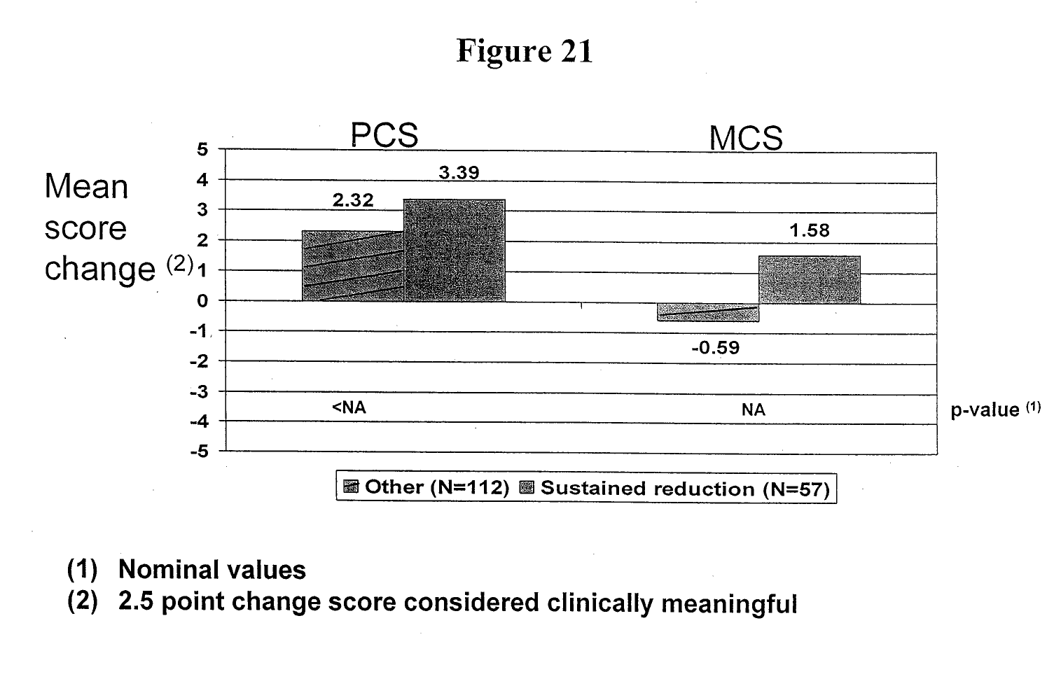

In addition, the PCS and MCS scores favored the patients of the Phase II/III study having a sustained reduction of anti-dsDNA antibodies over the others of the Phase II/III study. The mean PCS and MCS score changes for the sustained reduction population and the others of Phase II/III are shown in FIG. 21.

### Example 16

### HRQOL Following Renal Flare (Phase II/III and Phase III Studies)

The incidences of renal flares in patients of both sustained reduction group and the other group were documented and the results are shown in Table 7 for Phase III and

Table 8 for Phase II/III.

TABLE 7
Phase III patients with renal flares

| | |
|---|---|
| Sustained reduction | Other |

| | # | # Renal | # | # Renal |
|---|---|---|---|---|
| | Patients | flares | Patients | flares |
| LJP 394 | 80 | 3 | 65 | 14 |
| Placebo | 41 | 2 | 112 | 22 |
| Total* | 121 | 5 | 177 | 36 |

*Fisher's exact test

TABLE 8
Phase II/III patients with renal flares

| | |
|---|---|
| Sustained reduction | Other |

| | # | # Renal | # | # Renal |
|---|---|---|---|---|
| | Patients | flares | Patients | flares |
| LJP 394 | 54 | 1 | 38 | 6 |
| Placebo | 13 | 1 | 84 | 20 |
| Total* | 67 | 2 | 122 | 26 |

*Fisher's exact test

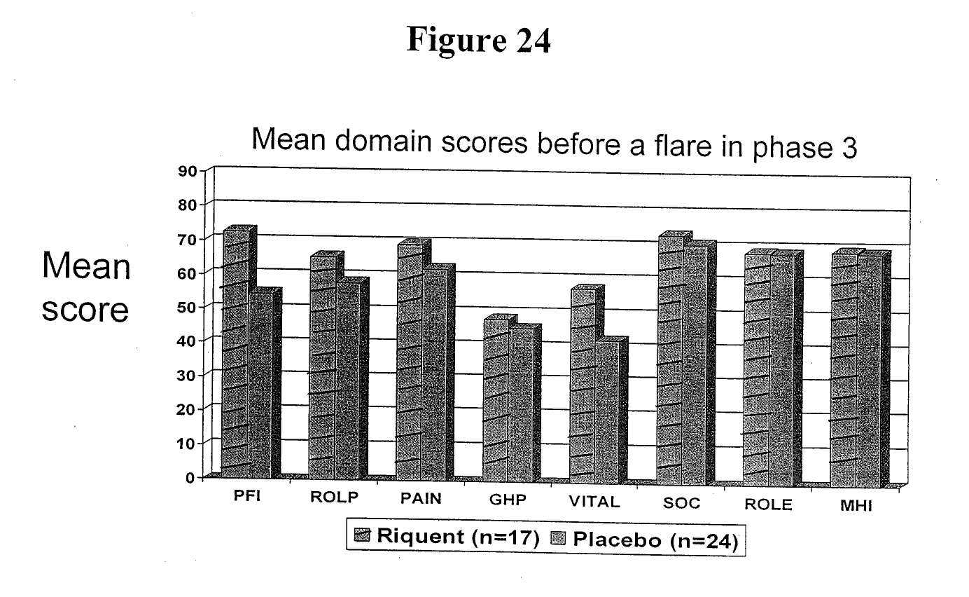

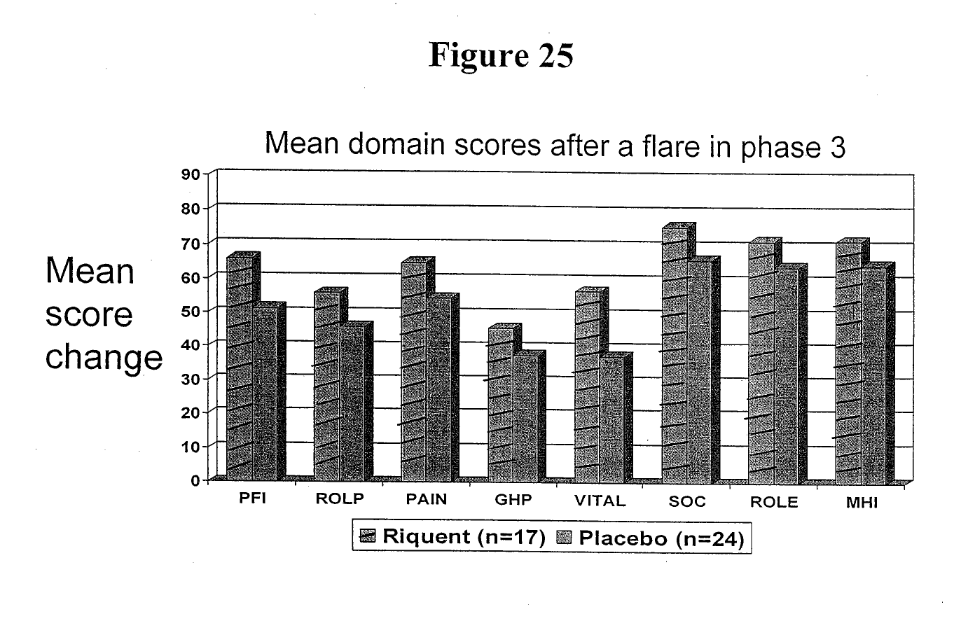

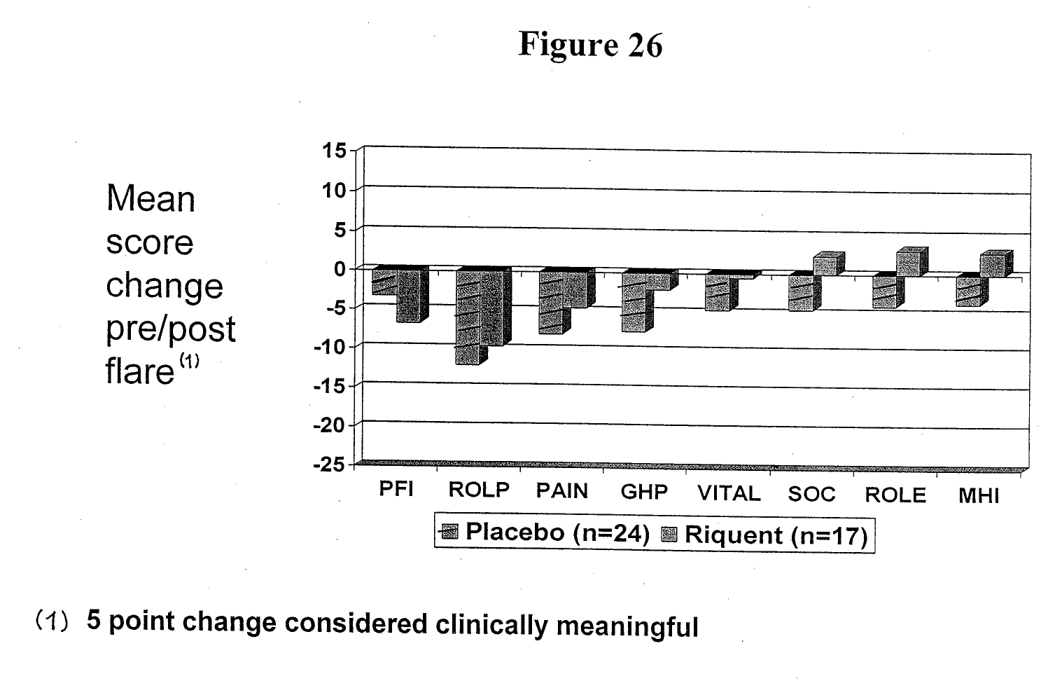

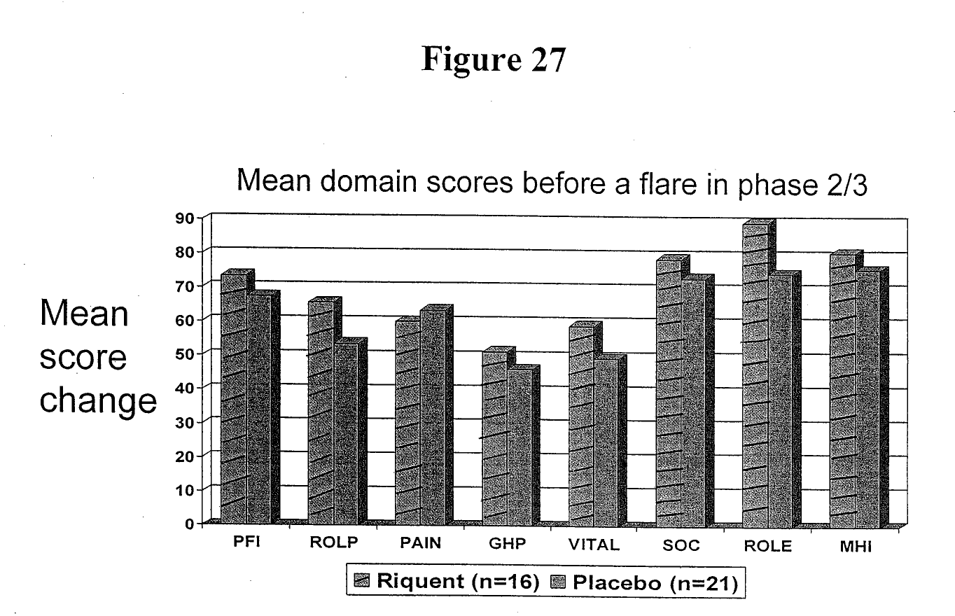

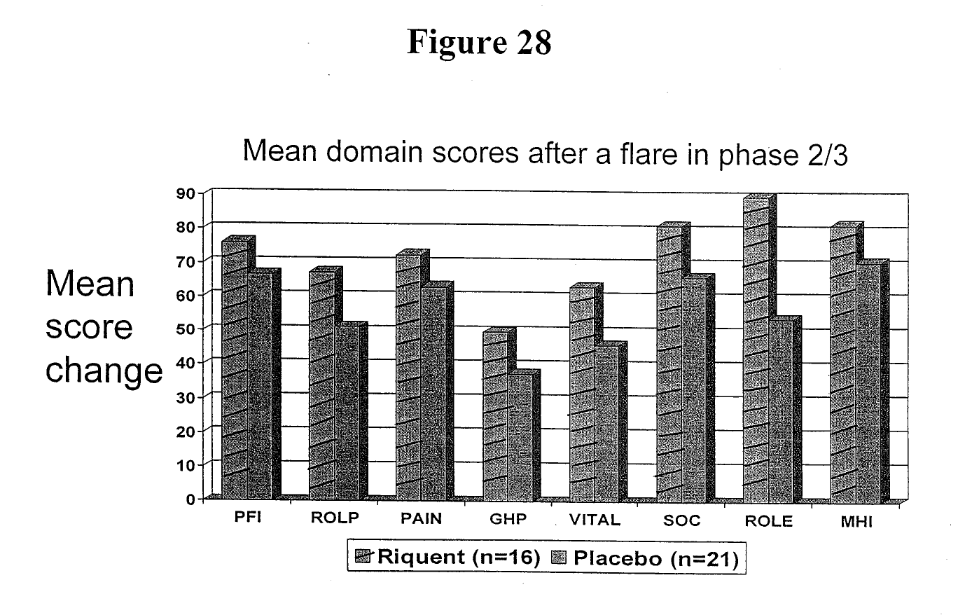

Data from the Phase III study also demonstrates that LJP394-treated patients reported improved HRQOL following a renal flare compared with placebo-treated patients. Following a renal flare, seven of eight domains of the SF-36® survey (all but physical functioning) were more favorable for LJP394-treated patients compared with placebo-treated patients (FIG. 26). Similar results were seen in the Phase II/III trial where all 8 domains were more favorable for LJP394-treated patients following a renal flare compared with placebo-treated patients (FIG. 8). The differences were not statistically significant due to the small number of total renal flares in each trial. Nonetheless, these data indicate that patients treated with LJP394 demonstrated less deterioration, stabilization or even improvement in most SF-36 domains while those receiving placebo reported deterioration in all domains. Patients receiving LJP394 had improved HRQOL scores before the renal flare compared with placebo in the phase III trial (FIG. 24) and all but the domain Pain (63.48 for placebo and 60.13 for LJP394) in the phase II/III trial (FIG. 27), as measured by mean scores at each SF 36® domain. All SF 36® domain values for LJP394 patients remained better than placebo patients after a renal flare in both Phase III and Phase II/III studies (FIGS. 25 and 28, respectively). These differences in HRQOL scores were observed despite standard-of-care therapies of immunosuppressive drugs.

### Example 17

### Longitudinal Effects on HRQOL (Phase II/III and Phase III Studies

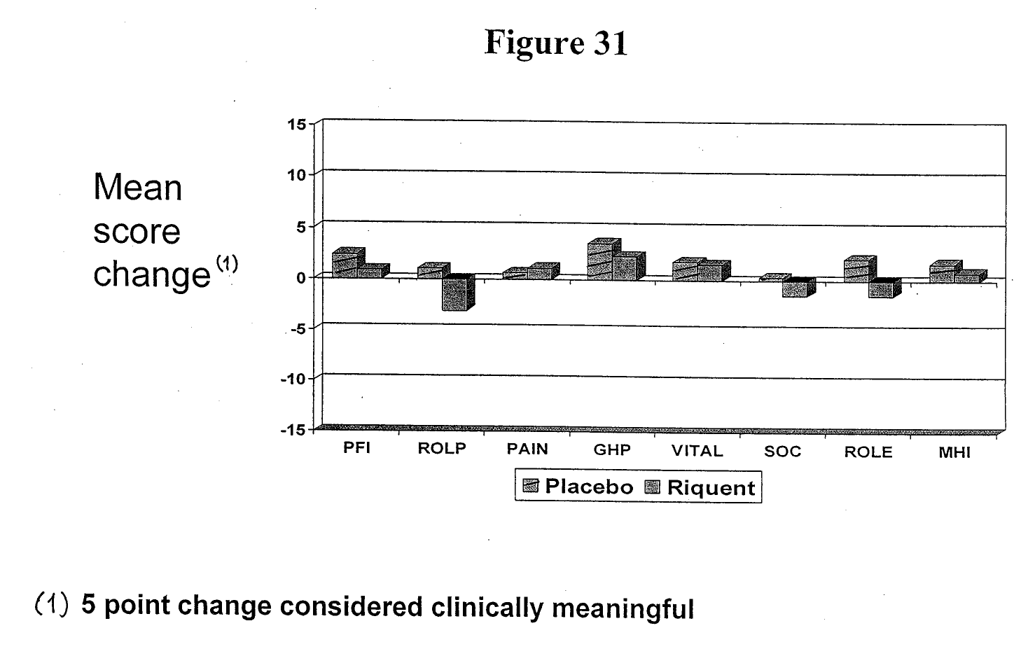

Other results from the Phase III trial indicate that longitudinal differences in HRQOL between the drug-treated and placebo-treated groups at various times during the trial were not significantly different (FIG. 31). These results largely mirror those of the Phase II/III study, except for the data corresponding to the Role Emotional, Role Physical and/or Social Functioning domains. The lack of significance may have been due to changes in medical practice during the trial and a loss of susceptible patients. In addition, too few SF-36 measurements may have been taken. Only two SF-36 measurements were recorded up to week 48 in the Phase II study. In the Phase II/III study, the analysis was limited to the “induction period” (the first 16 weeks of treatment). Only two SF 36 measurements were recorded during the induction period of the Phase II/III study.

Changes in medical practice: There appears to have been changes in medical practice since the completion of the Phase II/III study as evidenced by a difference in prescribing regimens for immunosuppressive drugs. In particular, it appears there were differences in baseline treatments in the patient population in the Phase III study compared with the previous trial. A higher percentage of patients were receiving immunosuppressive treatments at study entry: 73/145 (50%) in the LJP394-treated group versus 63/153 (41%) in the placebo-treated group in the Phase III study, compared with 35/114 (31%) in the LJP394-treated group versus 40/116 (34%) in the placebo-treated group in the Phase II/III trial. The sample size selected for the Phase III study was based on the Phase II/III study.

The definition of HDCC may not have captured all of the potential events in this study, as HDCC did not include some of the newer immunosuppressive drugs that are increasingly used instead of cyclophosphamide. While these newer drugs have a better side effect profile than cyclophosphamide, they are still broadly immunosuppressive. The definition of Major SLE flare included increases in corticosteroid doses as well as any new or increased dose of immunosuppressive agents, hospitalization or death, provided they were associated with active SLE.

Depletion of susceptible patients in placebo group: It appears that “sicker” patients in the LJP394 group stayed in the trial longer than “sicker” patients in the placebo group even though a comparable number of patients discontinued in each group before they met a predefined endpoint in the study (a depletion of susceptible patients). Reviewing a graph of the results showed that the LJP394 and placebo lines for time to renal flare and for the changes in antibody levels were separating until weeks 46 to 48. Upon reviewing patient laboratory values, placebo patients remaining in the study past weeks 44 to 48 appeared to have better renal function than the placebo group who dropped out or flared prior to these weeks, as measured by creatinine clearance at baseline (p=0.024 at week 48). In the placebo-treated group, those who remained in the study after weeks 44 to 48 showed no mean changes in antibodies to dsDNA from baseline.

### Example 18

### HRQOL of Patients with Sustained Reductions Following Renal Flares

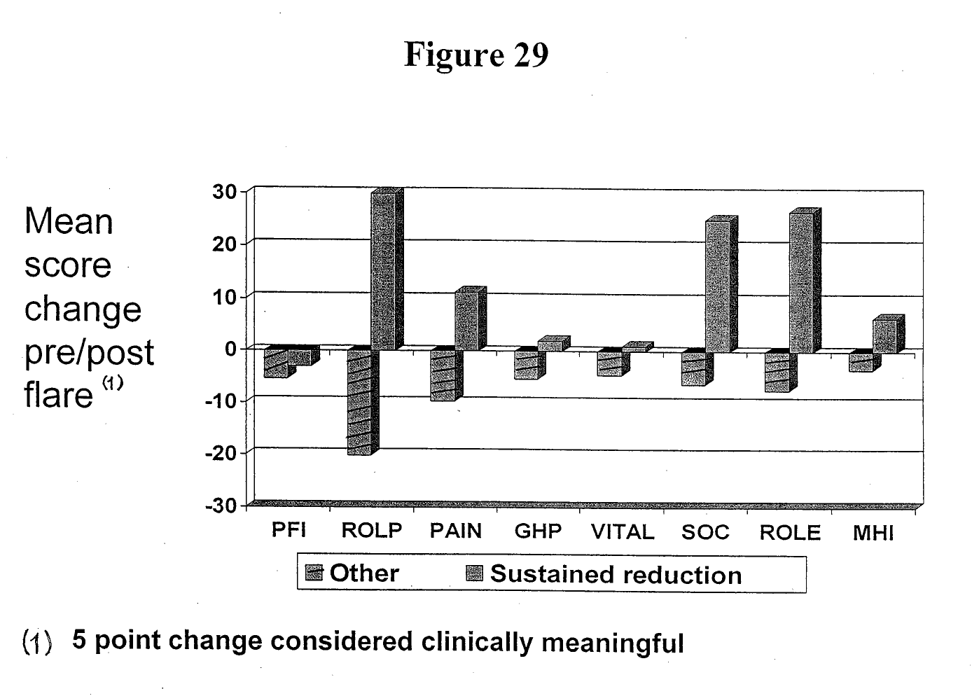

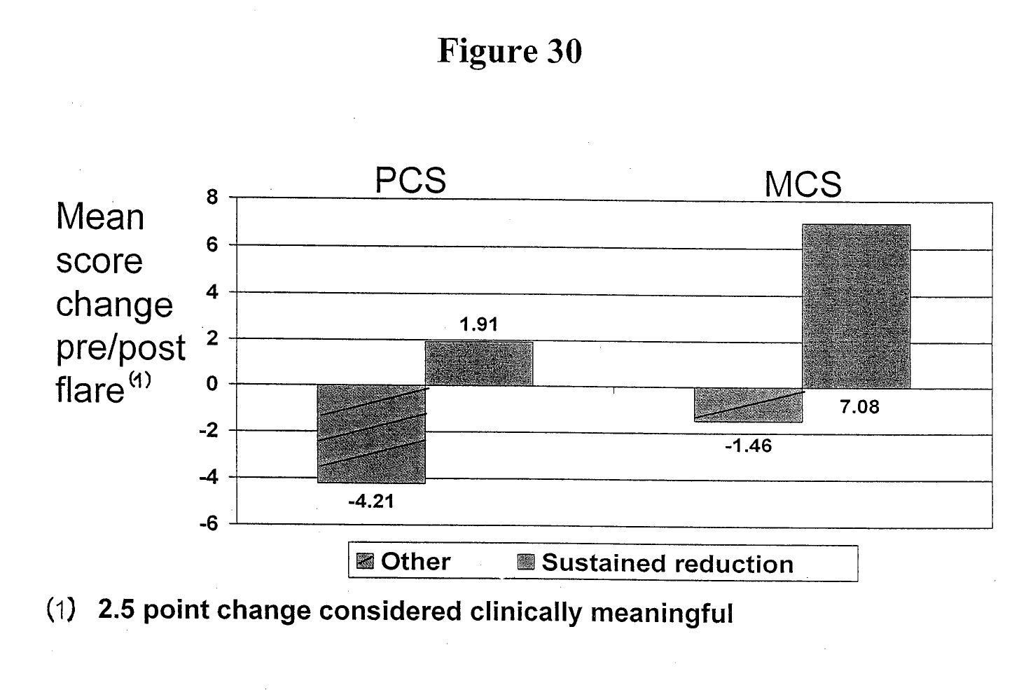

Phase III patients with sustained reductions of anti-dsDNA antibodies had better mean SF-36 domain score changes pre/post flare than the other population. These data are shown in FIG. 29. Phase III patients with sustained reductions of anti-dsDNA antibodies were also shown to have a better PCS and MCS mean score change pre/post flare (FIG. 30).

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention.

What is claimed is:

1. A method of stabilizing or improving the health-related quality of life of an individual with systemic lupus erythematosus (SLE), comprising administering to the individual an effective amount of a dsDNA epitope which specifically binds to an anti-dsDNA antibody from the individual, wherein the administration of the dsDNA epitope results in a stabilization of or improvement in the individual's health-related quality of life.

2. The method of claim 1, wherein administration of the dsDNA epitope results in a sustained reduction of the level of circulating anti-dsDNA antibodies in the individual.

3. The method of claim 2, wherein the sustained reduction is maintained for more than about 16 weeks.

4. The method of claim 3, wherein the sustained reduction is maintained for at least about 24 weeks.

5. The method of claim 1, wherein the dsDNA epitope comprises a double-stranded polynucleotide 5′-GTGTGTGTGTGTGTGTGTGT-3′(SEQ ID NO:1) in combination with its complementary strand, or one of the single-stranded polynucleotides 5′-GTGTGTGTGTGTGTGTGTGT-3′(SEQ ID NO:1) or 3′-CACACACACACACACACACA-5′(SEQ ID NO:2).

6. The method of claim 1, wherein the dsDNA epitope is administered in the form of a conjugate comprising (a) a non-immunogenic valency platform molecule and (b) two or more double-stranded DNA (dsDNA) epitopes that specifically bind to an anti-dsDNA antibody from the individual.

7. The method of claim 6, wherein the conjugate is a compound of the formula 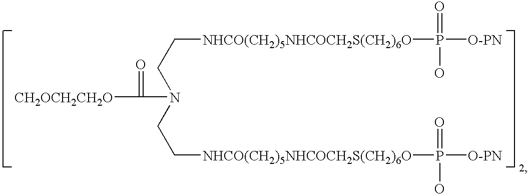
wherein PN is (CA)₁₀·(TG)₁₀.

8. The method of claim 1, wherein the stabilization or improvement in the individual's health-related quality of life occurs following a renal flare.

9. The method of claim 8, wherein the effective amount of the dsDNA epitope is administered to the individual for a period of more than about 16 weeks.

10. The method of claim 9, wherein the dsDNA epitope is administered in the form of a compound of the formula 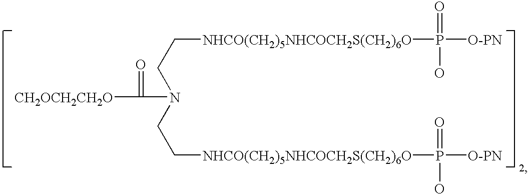
wherein PN is (CA)₁₀·(TG)₁₀.

11. The method of claim 1, wherein the stabilization or improvement in the individual's health-related quality of life is detectable by the Medical Outcome Survey Short Form 36 (SF-36), wherein the stabilization or improvement is reflected in one or more domain scores selected from the group consisting of physical functioning, role physical, bodily pain, general health perception, vitality, social functioning, role emotional, and mental health.

12. The method of claim 11, wherein the stabilization or improvement in the individual's health-related quality of life is detectable by the Medical Outcome Survey Short Form 36 (SF-36), wherein the stabilization or improvement is reflected in one or more domain scores selected from the group consisting of physical functioning, role physical, bodily pain, general health perception, vitality, social functioning, and mental health.

13. The method of claim 1, which is a method of stabilizing the health-related quality of life of an individual with systemic lupus erythematosus (SLE), wherein the administration of the dsDNA epitope results in a stabilization of the individual's health-related quality of life.

14. The method of claim 1, which is a method of improving the health-related quality of life of an individual with systemic lupus erythematosus (SLE), wherein the administration of the dsDNA epitope results in an improvement in the individual's health-related quality of life.

15. The method of claim 1, wherein the individual is a human.

16. The method of claim 1, wherein the effective amount of the dsDNA epitope is administered to the individual for a period of more than about 16 weeks.

17. The method of claim 16, wherein the dsDNA epitope is administered in the form of a compound of the formula 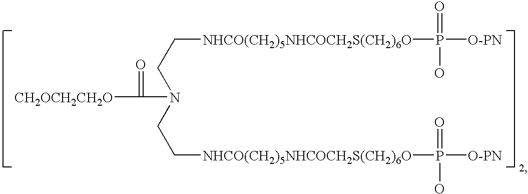
wherein PN is (CA)₁₀·(TG)₁₀.

18. A method of stabilizing or improving the health-related quality of life of an individual with systemic lupus erythematosus (SLE), comprising reducing the level of circulating anti-dsDNA antibodies in the individual, wherein the reduction of the level of circulating anti-dsDNA antibodies in the individual results in a stabilization or improvement of the health-related quality of life in the individual.

19. The method of claim 18, wherein a sustained reduction of circulating anti-dsDNA antibodies in the individual is achieved.

20. The method of claim 19, wherein the sustained reduction is maintained for more than about 16 weeks.

21. The method of claim 20, wherein the stabilization or improvement in the health-related quality of life of the individual occurs following a renal flare.

22. The method of claim 21, wherein the sustained reduction is effected by administration to the individual of an effective amount of a dsDNA epitope which specifically binds to an anti-dsDNA antibody from the individual.

23. The method of claim 22, wherein the sustained reduction is effected by administration of an effective amount of a compound of the formula 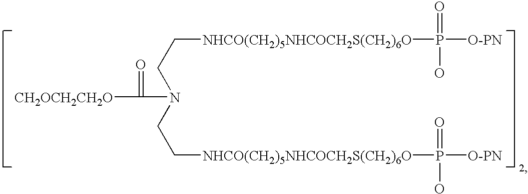
wherein PN is (CA)₁₀·(TG)₁₀.

24. The method of claim 20, wherein the sustained reduction is maintained for at least about 24 weeks.

25. The method of claim 18, wherein the step of reducing the level of circulating anti-dsDNA antibodies comprises administering to the individual an effective amount of an agent that reduces the level of circulating anti-dsDNA antibodies in the individual.

26. The method of claim 25, wherein the agent comprises a dsDNA epitope which specifically binds to an anti-dsDNA antibody from the individual.

27. The method of claim 26, wherein the dsDNA epitope comprises a double-stranded polynucleotide 5′-GTGTGTGTGTGTGTGTGTGT-3′(SEQ ID NO: 1) and its complementary strand, or one of the single-stranded polynucleotides 5′-GTGTGTGTGTGTGTGTGTGT-3′(SEQ ID NO:1) or 3′-CACACACACACACACACACA-5′(SEQ ID NO:2).

28. The method of claim 26, wherein the agent comprises a conjugate comprising (a) a non-immunogenic valency platform molecule and (b) two or more double-stranded DNA (dsDNA) epitopes that specifically bind to an anti-dsDNA antibody from the individual.

29. The method of claim 28, wherein the conjugate is a compound of the formula 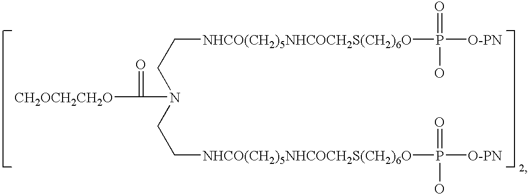
wherein PN is (CA)₁₀·(TG)₁₀.

30. The method of claim 18, wherein the stabilization or improvement in the individual's health-related quality of life is detectable by the Medical Outcome Survey Short Form 36 (SF-36), wherein the stabilization or improvement is reflected in one or more domains of health-related quality of life selected from the group consisting of physical functioning, role physical, bodily pain, general health perception, vitality, social functioning, role emotional, and mental health.

31. The method of claim 18, wherein the stabilization or improvement in the individual's health-related quality of life occurs following a renal flare.

32. The method of claim 31, wherein the step of reducing the level of circulating anti-dsDNA antibodies in the individual comprises administering to the individual an effective amount of an agent that reduces anti-dsDNA antibodies in the individual.

33. The method of claim 32, wherein the agent comprises a dsDNA epitope which specifically binds to an anti-dsDNA antibody from the individual.

34. The method of claim 33, wherein the dsDNA epitope comprises a double-stranded polynucleotide 5′-GTGTGTGTGTGTGTGTGTGT-3′(SEQ ID NO:1) and its complementary strand, or one of the single-stranded polynucleotides 5′-GTGTGTGTGTGTGTGTGTGT-3′(SEQ ID NO:1) or 3′-CACACACACACACACACACA-5′(SEQ ID NO:2).

35. The method of claim 33, wherein the agent comprises a conjugate comprising (a) a non-immunogenic valency platform molecule and (b) two or more double-stranded DNA (dsDNA) epitopes that specifically bind to an anti-dsDNA antibody from the individual.

36. The method of claim 35, wherein the conjugate is a compound of the formula 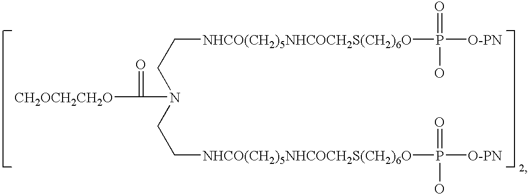
wherein PN is (CA)₁₀·(TG)₁₀.

37. The method of claim 31, wherein a sustained reduction of circulating anti-dsDNA antibodies in the individual is achieved.

38. The method of claim 31, wherein the stabilization or improvement in the individual's health-related quality of life is detectable by the Medical Outcome Survey Short Form 36 (SF-36), wherein the stabilization or improvement is reflected in one or more domain scores selected from the group consisting of physical functioning, role physical, bodily pain, general health perception, vitality, social functioning, role emotional, and mental health.

39. A method of stabilizing or improving the health-related quality of life in an individual with SLE comprising the steps of:
(a) selecting an individual for receiving or continuing to receive treatment based on the individual's need for a stabilized or improved health-related quality of life; and
(b) administering a treatment to the selected individual, wherein administration of the treatment effects a sustained reduction of anti-dsDNA antibodies in the individual.

40. A method of stabilizing or improving the health-related quality of life in an individual having SLE comprising the steps of:
(a) selecting an individual to receive or continue to receive a dsDNA epitope based on the affinity of the dsDNA epitope for an anti-dsDNA antibody in the individual; and
(b) administering the dsDNA epitope to the selected individual, wherein administration of the dsDNA epitope stabilizes or improves the health-related quality of life in an individual.
